SEQUENCE LISTING (34 sequences)

SEQ ID NO: 1 - DNA, 20 bp, Artificial Sequence
  Description of Artificial Sequence Synthetic primer
gctgctttgcctacctctcc

SEQ ID NO: 2 - DNA, 21 bp, Artificial Sequence
  Description of Artificial Sequence Synthetic primer
tcgagtgacaaacacgactgc

SEQ ID NO: 3 - DNA, 21 bp, Artificial Sequence
  Description of Artificial Sequence Synthetic primer
ccaagtgctgccgtcattttc

SEQ ID NO: 4 - DNA, 21 bp, Artificial Sequence
  Description of Artificial Sequence Synthetic primer
ggctcgcagggatgatttcaa

SEQ ID NO: 5 - DNA, 21 bp, Artificial Sequence
  Description of Artificial Sequence Synthetic primer
aaagaagcagattgcccagaa

SEQ ID NO: 6 - DNA, 20 bp, Artificial Sequence
  Description of Artificial Sequence Synthetic primer
tctgcgtcagaaagacctca

SEQ ID NO: 7 - DNA, 21 bp, Artificial Sequence
  Description of Artificial Sequence Synthetic primer
aggtcggtgtgaacggatttg

SEQ ID NO: 8 - DNA, 23 bp, Artificial Sequence
  Description of Artificial Sequence Synthetic primer
tgtagaccatgtagttgaggtca

SEQ ID NO: 9 - DNA, 23 bp, Artificial Sequence
  Description of Artificial Sequence Synthetic primer
cagttgacaatgcctttggaatg

SEQ ID NO: 10 - DNA, 22 bp, Artificial Sequence
  Description of Artificial Sequence Synthetic primer
cactctatcacaggaggtctgg

SEQ ID NO: 11 - DNA, 20 bp, Artificial Sequence
  Description of Artificial Sequence Synthetic oligonucleotide
acgcaaagatatctcggagg

SEQ ID NO: 12 - DNA, 20 bp, Artificial Sequence
  Description of Artificial Sequence Synthetic oligonucleotide
gcgagggtccaggaaggaac

SEQ ID NO: 13 - DNA, 22 bp, Artificial Sequence
  Description of Artificial Sequence Synthetic oligonucleotide
ttcatattcaattttcttcgat

SEQ ID NO: 14 - DNA, 22 bp, Artificial Sequence
  Description of Artificial Sequence Synthetic oligonucleotide
ttcataatattcttgtagctca

SEQ ID NO: 15 - DNA, 20 bp, Artificial Sequence
  Description of Artificial Sequence Synthetic oligonucleotide
gaaggccaaacatccaactg

SEQ ID NO: 16 - DNA, 20 bp, Artificial Sequence
  Description of Artificial Sequence Synthetic oligonucleotide
ctacataacaacatgctcag

SEQ ID NO: 17 - DNA, 22 bp, Artificial Sequence
  Description of Artificial Sequence Synthetic oligonucleotide
tatataaaatcctttggctggg

SEQ ID NO: 18 - DNA, 22 bp, Artificial Sequence
  Description of Artificial Sequence Synthetic oligonucleotide
ttctacatatataaaatccttt

SEQ ID NO: 19 - DNA, 20 bp, Artificial Sequence
  Description of Artificial Sequence Synthetic oligonucleotide
tacaacgcaagttgccactg

SEQ ID NO: 20 - DNA, 20 bp, Artificial Sequence
  Description of Artificial Sequence Synthetic oligonucleotide
gattccggataaagtcccta

SEQ ID NO: 21 - DNA, 20 bp, Artificial Sequence
  Description of Artificial Sequence Synthetic oligonucleotide
ggtgaccgctaatcatcagg

SEQ ID NO: 22 - DNA, 20 bp, Artificial Sequence
  Description of Artificial Sequence Synthetic oligonucleotide
gattaccgtgatctgaaatg

SEQ ID NO: 23 - DNA, 20 bp, Artificial Sequence
  Description of Artificial Sequence Synthetic oligonucleotide
gaagtctataacttaatgtg

SEQ ID NO: 24 - DNA, 22 bp, Artificial Sequence
  Description of Artificial Sequence Synthetic oligonucleotide
ttaataatcttctcttctgcca

SEQ ID NO: 25 - DNA, 22 bp, Artificial Sequence
  Description of Artificial Sequence Synthetic oligonucleotide
tttcaataaaaaatcattccac

SEQ ID NO: 26 - DNA, 22 bp, Artificial Sequence
  Description of Artificial Sequence Synthetic oligonucleotide
ttagagacaattatattccgta

SEQ ID NO: 27 - DNA, 22 bp, Artificial Sequence
  Description of Artificial Sequence Synthetic oligonucleotide
tattaaataattttgagttgta

SEQ ID NO: 28 - DNA, 30 bp, Mus musculus
tctttccttagggactttatccggaatccc

SEQ ID NO: 29 - DNA, 29 bp, Artificial Sequence
  Description of Artificial Sequence Synthetic oligonucleotide
tctttccttaggactttatccggaatccc

SEQ ID NO: 30 - DNA, 30 bp, Artificial Sequence
  Description of Artificial Sequence Synthetic oligonucleotide
tctttccttaggggactttatccggaatcc

SEQ ID NO: 31 - DNA, 30 bp, Artificial Sequence
  Description of Artificial Sequence Synthetic oligonucleotide
tctttccttaaggactttatccggaatccc

SEQ ID NO: 32 - DNA, 17 bp, Artificial Sequence
  Description of Artificial Sequence Synthetic oligonucleotide
tctttatccggaatccc

SEQ ID NO: 33 - DNA, 30 bp, Artificial Sequence
  Description of Artificial Sequence Synthetic oligonucleotide
tctttcctttgtgactttatccggaatccc

SEQ ID NO: 34 - DNA, 27 bp, Artificial Sequence
  Description of Artificial Sequence Synthetic oligonucleotide
tctttcctggactttatccggaatccc

(19) United States
(12) Patent Application Publication
(10) Pub. No.: US 2023/0324392 A1
(43) Pub. Date: Oct. 12, 2023

(54) METHODS AND COMPOSITIONS FOR TARGETING CYTOSOLIC DSDNA SIGNALING IN CHROMOSOMALLY UNSTABLE CANCERS

(71) Applicant: MEMORIAL SLOAN KETTERING CANCER CENTER, New York, NY (US)

(72) Inventors: Samuel Bakhoum, New York, NY (US); Jun Li, New York, NY (US)

(21) Appl. No.: 18/043,527

(22) PCT Filed: Sep. 1, 2021
(86) PCT No.: PCT/US21/48743

§ 371 Date: Feb. 28, 2023

Related U.S. Application Data
(60) Provisional application No. 63/073,621, filed on Sep. 2, 2020.

(51) Int. Cl. G01N 33/574; A61K 45/06; A61P 35/00

(52) U.S. Cl. CPC G01N 33/574; A61K 45/06; G01N 2333/4706; G01N 2800/52; A61P 35/00

(57) ABSTRACT

The present technology provides methods and compositions for identifying and treating cancers by targeting the cytosolic dsDNA sensing pathway (cGAS-STING) in chromosomally unstable cancers. In some embodiments, the present technology also provides methods for detecting chromosomal instability in cancer and treating cancers associated with altered levels of cyclic GMP-AMP synthase (cGAS), stimulator of interferon genes (STING), and/or ectonucleotide pyrophosphatase/phosphodiesterase 1 (ENPP1).

### CROSS-REFERENCE TO RELATED APPLICATION

The present application claims priority to U.S. Provisional Patent Application No. 63/073,621, filed on Sep. 2, 2020, the contents of which are hereby incorporated by reference in their entirety.

### U.S. GOVERNMENT LICENSE RIGHTS

This invention was made with government support under OD026395 awarded by the National Institutes of Health. The government has certain rights in the invention.

### TECHNICAL FIELD

The present technology relates generally to methods for identifying and treating cancers by targeting the cytosolic dsDNA sensing pathway (cGAS-STING) in chromosomally unstable cancers. In particular, the present technology relates to methods for detecting chromosomal instability in cancer and treating cancers associated with altered levels of cyclic GMP-AMP synthase (cGAS), stimulator of interferon genes (STING), and/or ectonucleotide pyrophosphatase/phosphodiesterase 1 (ENPP1).

### BACKGROUND

Chromosomal instability (CIN) is a hallmark of human cancer and it is associated with widespread resistance, immune evasion, and metastasis. Chromosome segregation errors lead to the formation of micronuclei. Micronuclear envelopes are highly rupture-prone, often exposing genomic double-stranded DNA (dsDNA) to the cytosol. Cytosolic dsDNA is sensed by cGAS, which upon binding to its substrate, catalyzes the formation of cyclic dinucleotide, cGAMP. cGAMP is a potent immune-stimulatory molecule that promotes inflammatory signaling in a manner dependent on its downstream effector, STING. Chromosomally unstable cancer cells have evolved to cope with chromic cGAS-STING activation by silencing downstream type I interferon signaling whilst co-opting NF-κB-dependent transcription to spread to distant organs. However, cGAMP is also readily exported to the extracellular space where it can promote anti-tumor immune responses by activating STING in neighboring host cells. How cancer cells co-opt inflammatory signaling while simultaneously evading immune surveillance remains unknown. Furthermore, the role of extracellular cGAMP in metastasis and immune evasion in the context of CIN remains poorly understood. Because cancer cells rely in large part on CIN and its downstream inflammatory signaling to spread to distant organs, identifying mechanisms by which tumor cells cope with inflammation represents an attractive therapeutic opportunity. Unlike many targetable genomic alterations in cancer, CIN remains a major therapeutic challenge given the lack of defined pathways that are shared among chromosomally unstable tumors. Accordingly, there is a need to identify targets in the cytosolic DNA-sensing cGAS-STING pathway to develop personalized therapies for the treatment of cancer.

### SUMMARY

In one aspect, the disclosure of the present technology provides a method for detecting chromosomal instability in a tumor in a subject, comprising: obtaining one or more tissue sections of a tumor sample from a subject; and measuring the degree of chromosomal instability present in the tumor sample by contacting the tumor sample with an anti-cGAS antibody and measuring the presence of cGAS⁺ micronuclei in the tumor sample.

In some embodiments, measuring the presence of cGAS⁺ micronuclei in the tumor sample comprises one or more of: quantifying the number of cGAS⁺ micronuclei in a defined high-power field, wherein a high degree of chromosomal instability in the tumor is detected when 5 or more cGAS⁺ micronuclei are present in the high-power field; and quantifying the fraction of cGAS⁺ micronuclei/primary nuclei, wherein a high degree of chromosomal instability in the tumor is detected when the fraction of cGAS⁺ micronuclei/primary nuclei is 8% or higher.

In some embodiments, the measuring of the presence of cGAS⁺ micronuclei in the tumor sample comprises quantifying the fraction of cGAS⁺ micronuclei/primary nuclei, wherein a high degree of chromosomal instability in the tumor is detected when the fraction of cGAS⁺ micronuclei/primary nuclei is 10% or higher.

In one aspect, the disclosure of the present technology provides a method for treating cancer associated with increased cGAS⁺ micronuclei and decreased stimulator of interferon genes (STING) protein expression (cGAS^{high}STING^{low}), in a subject in need thereof, comprising: detecting an increase in cGAS⁺ micronuclei and a decrease in cancer cell-specific STING protein expression in a tumor sample (cGAS^{high}STING^{low} tumor) obtained from a subject as compared to that observed in a reference sample, thereby detecting a cGAS^{high}STING^{low} tumor; and administering a therapeutically effective amount of a STING inhibitor to the subject for whom a cGAS^{high}STING^{low} tumor has been detected.

In one aspect, the disclosure of the present technology provides a method for selecting a subject for the treatment of cancer with a stimulator of interferon genes (STING) inhibitor, comprising: detecting an increase in cGAS⁺ micronuclei and a decrease in STING protein expression in a tumor sample (cGAS^{high}STING^{low} tumor) obtained from a subject as compared to that observed in a reference sample; and selecting the subject for whom a cGAS^{high}STING^{low} tumor sample has been detected as a subject for the treatment of cancer with a STING inhibitor.

In some embodiments, the reference sample is obtained from a healthy control subject, or normal tissue corresponding to the tumor sample, or contains a predetermined level of cGAS⁺ micronuclei and STING protein expression.

In some embodiments, detecting cGAS⁺ micronuclei comprises contacting the tumor sample with an anti-cGAS antibody and measuring the presence of cGAS⁺ micronuclei in the tumor sample, and detecting cancer cell-specific STING protein expression comprises contacting the tumor sample with an anti-STING antibody and measuring STING expression levels in the tumor sample.

In some embodiments, the STING inhibitor is selected from the group consisting of C-176, C-178, compound H-151, tetrahydroisoquinolone acetic acids, 9-nitrooleate, 10-nitrooleate, nitro conjugated linoleic acid, nitrofurans, Astin C, Astin C analogue M11, and any combination thereof.

In some embodiments, the methods further comprise separately, sequentially, or simultaneously administering to the subject one or more immune checkpoint blocking agents selected from the group consisting of anti-PD-L1 antibody, anti-PD-1 antibody, anti-CTLA-4 antibody, ipilimumab, nivolumab, pidilizumab, lambrolizumab, pembrolizumab, atezolizumab, avelumab, durvalumab, MPDL3280A, BMS-936559, MEDI-4736, MSB 00107180, LAG-3, TIM3, B7-H3, B7-H4, TIGIT, AMP-224, MDX-1105, arelumab, tremelimumab, IMP321, MGA271, BMS-986016, lirilumab, urelumab, PF-05082566, IPH2101, MEDI-6469, CP-870,893, Mogamulizumab, Varlilumab, Galiximab, AMP-514, AUNP 12, Indoximod, NLG-919, INCB024360, CD80, CD86, ICOS, DLBCL inhibitors, BTLA, PDR001, and any combination thereof.

In some embodiments, the cancer is a solid malignant tumor. In some embodiments, the solid malignant tumor is selected from the group consisting of melanoma, breast cancer, colorectal cancer, lung cancer, prostate cancer, bladder cancer, pancreatic cancer, ovarian cancer, squamous cell carcinoma of the skin, Merkel cell carcinoma, gastric cancer, liver cancer, thyroid cancer, and sarcoma.

In some embodiments, treatment comprises increasing survival, decreasing metastasis, reducing tumor burden, reducing tumor relapse during post-debulking adjuvant chemotherapy in the subject, reducing the number of cancer cells, reducing the tumor size, eradicating the tumor, inhibiting cancer cell infiltration into peripheral organs, inhibiting or stabilizing tumor growth, and/or stabilizing or improving quality of life in the subject.

In some embodiments, the subject is a mammal. In some embodiments, the mammalian subject is a human.

In one aspect, the disclosure of the present technology provides a method for increasing tumor sensitivity to treatment with a stimulator of interferon genes (STING) agonist in a subject in need thereof, comprising: detecting an increase in cGAS⁺ micronuclei and a decrease in STING protein expression in a tumor sample (cGAS^{high}STING^{low} tumor) obtained from a subject as compared to that observed in a reference sample; and administering a therapeutically effective amount of a cGAS inhibitor to the subject for whom a cGAS^{high}STING^{low} tumor has been detected prior to administering a therapeutically effective amount of a STING agonist to the subject.

In one aspect, the disclosure of the present technology provides a method for treating cancer having increased cGAS⁺ micronuclei and decreased stimulator of interferon genes (STING) protein expression in cancer cells (cGAS^{high}STING^{low}), in a subject in need thereof, comprising: detecting an increase in cGAS⁺ micronuclei and a decrease in cancer cell-specific STING protein expression in a tumor sample (cGAS^{high}STING^{low} tumor) obtained from a subject as compared to that observed in a reference sample, thereby detecting a cGAS^{high}STING^{low} tumor; and administering a therapeutically effective amount of a cGAS inhibitor to the subject for whom a cGAS^{high}STING^{low} tumor has been detected; and subsequently administering a STING agonist to the subject for whom a cGAS^{high}STING^{low} tumor has been detected.

In some embodiments, the reference sample is obtained from a healthy control subject, or normal tissue corresponding to the tumor sample, or contains a predetermined level of cGAS⁺ micronuclei and STING protein expression.

In some embodiments, detecting cGAS⁺ micronuclei comprises contacting the tumor sample with an anti-cGAS antibody and measuring the presence of cGAS⁺ micronuclei in the tumor sample, and detecting cancer cell-specific STING protein expression comprises contacting the tumor sample with an anti-STING antibody and measuring STING expression levels in the tumor sample.

In some embodiments, the cGAS inhibitor is selected from the group consisting of J014 or analogs thereof, G150 or analogs thereof, RU.521, suramin, PF-06928215, hydroxychloroquine, quinacrin, and any combination thereof.

In some embodiments, the STING agonist is selected from the group consisting of c-di-AMP, c-di-GMP, diABZIs, 3′3′-cGAMP, 2′3′-cGAMP, 5,6-dimethylxanthenone-4-acetic acid (DMXAA), macrocycle-bridged STING agonist E7766, GSK3745417, MK-1454, MK-2118, ADU-S100, SB11285, BMS-98630, and any combination thereof.

In some embodiments, the STING agonist is administered within about 0 hours, within less than 1, or within about 24 hours, about 48 hours, about 72 hours, about one week, or about two weeks or more of the cGAS inhibitor.

In some embodiments, the methods further comprise separately, sequentially, or simultaneously administering to the subject: (i) one or more immune checkpoint blocking agents selected from the group consisting of anti-PD-L1 antibody, anti-PD-1 antibody, anti-CTLA-4 antibody, ipilimumab, nivolumab, pidilizumab, lambrolizumab, pembrolizumab, atezolizumab, avelumab, durvalumab, MPDL3280A, BMS-936559, MEDI-4736, MSB 00107180, LAG-3, TIM3, B7-H3, B7-H4, TIGIT, AMP-224, MDX-1105, arelumab, tremelimumab, IMP321, MGA271, BMS-986016, lirilumab, urelumab, PF-05082566, IPH2101, MEDI-6469, CP-870,893, Mogamulizumab, Varlilumab, Galiximab, AMP-514, AUNP 12, Indoximod, NLG-919, INCB024360, CD80, CD86, ICOS, DLBCL inhibitors, BTLA, PDR001, and any combination thereof, (ii) radiation therapy; and/or (iii) chemotherapy.

In some embodiments, the cancer is a solid malignant tumor. In some embodiments, the solid malignant tumor is selected from the group consisting of melanoma, breast cancer, colorectal cancer, lung cancer, prostate cancer, bladder cancer, pancreatic cancer, ovarian cancer, squamous cell carcinoma of the skin, Merkel cell carcinoma, gastric cancer, liver cancer, thyroid cancer, and sarcoma.

In some embodiments, treatment comprises increasing survival, decreasing local tumor recurrence, decreasing metastasis, reducing tumor burden, reducing tumor relapse during post-debulking adjuvant chemotherapy in the subject, reducing the number of cancer cells, reducing the tumor size, eradicating the tumor, inhibiting cancer cell infiltration into peripheral organs, inhibiting or stabilizing tumor growth, and/or stabilizing or improving quality of life in the subject.

In some embodiments, the subject is a mammal. In some embodiments, the mammalian subject is a human.

In one aspect, the disclosure of the present technology provides a method for treating cancer associated with decreased cGAS⁺ micronuclei and increased stimulator of interferon genes (STING) protein expression (cGAS^{low}STING^{high}), in a subject in need thereof, comprising: detecting a decrease in cGAS⁺ micronuclei and an increase in cancer cell-specific STING protein expression in a tumor sample (cGAS^{low}STING^{high} tumor) obtained from a subject as compared to that observed in a reference sample, thereby detecting a cGAS^{low}STING^{high} tumor; and administering one or more cancer therapies selected from radiation therapy, chemotherapy, and immunotherapy to the subject for whom a cGAS^{low}STING^{high} tumor has been detected.

In one aspect, the disclosure of the present technology provides a method for selecting a subject for the treatment of cancer with a stimulator of interferon genes (STING) inhibitor, comprising: detecting a decrease in cGAS⁺ micronuclei and an increase in STING protein expression in a tumor sample (cGAS^{low}STING^{high} tumor) obtained from a subject as compared to that observed in a reference sample; and selecting the subject for whom a cGAS^{low}STING^{high} tumor sample has been detected as a subject for the treatment of cancer with one or more of radiation therapy, chemotherapy, and immunotherapy.

In some embodiments, the immunotherapy comprises administering to the subject an immune checkpoint blocking agent selected from the group consisting of anti-PD-L1 antibody, anti-PD-1 antibody, anti-CTLA-4 antibody, ipilimumab, nivolumab, pidilizumab, lambrolizumab, pembrolizumab, atezolizumab, avelumab, durvalumab, MPDL3280A, BMS-936559, MEDI-4736, MSB 00107180, LAG-3, TIM3, B7-H3, B7-H4, TIGIT, AMP-224, MDX-1105, arelumab, tremelimumab, IMP321, MGA271, BMS-986016, lirilumab, urelumab, PF-05082566, IPH2101, MEDI-6469, CP-870,893, Mogamulizumab, Varlilumab, Galiximab, AMP-514, AUNP 12, Indoximod, NLG-919, INCB024360, CD80, CD86, ICOS, DLBCL inhibitors, BTLA, PDR001, and any combination thereof.

In some embodiments, the reference sample is obtained from a healthy control subject, or normal tissue corresponding to the tumor sample, or contains a predetermined level of cGAS⁺ micronuclei and STING protein expression.

In some embodiments, detecting cGAS⁺ micronuclei comprises contacting the tumor sample with an anti-cGAS antibody and measuring the presence of cGAS⁺ micronuclei in the tumor sample, and detecting cancer cell-specific STING protein expression comprises contacting the tumor sample with an anti-STING antibody and measuring STING expression levels in the tumor sample.

In some embodiments, the cancer is a solid malignant tumor. In some embodiments, the solid malignant tumor is selected from the group consisting of melanoma, breast cancer, colorectal cancer, lung cancer, prostate cancer, bladder cancer, pancreatic cancer, ovarian cancer, squamous cell carcinoma of the skin, Merkel cell carcinoma, gastric cancer, liver cancer, thyroid cancer, and sarcoma.

In some embodiments, treatment comprises increasing survival, decreasing local tumor recurrence, decreasing metastasis, reducing tumor burden, reducing tumor relapse during post-debulking adjuvant chemotherapy in the subject, reducing the number of cancer cells, reducing the tumor size, eradicating the tumor, inhibiting cancer cell infiltration into peripheral organs, inhibiting or stabilizing tumor growth, and/or stabilizing or improving quality of life in the subject.

In some embodiments, the subject is a mammal. In some embodiments, the mammalian subject is a human.

In one aspect, the disclosure of the present technology provides a method for treating cancer associated with increased ENPP1 expression in a subject in need thereof, comprising: detecting the presence or absence of an increased ENPP1 protein expression level in a tumor sample obtained from a subject as compared to that observed in a reference sample; and administering a therapeutically effective amount of an ENPP1 inhibitor to the subject for whom an increased ENPP1 protein expression level has been detected.

In one aspect, the disclosure of the present technology provides a method for treating cancer associated with increased cGAS⁺ micronuclei and increased ENPP1 expression (cGAS^{high}ENPP1^{high}), in a subject in need thereof, comprising: detecting the presence or absence of an increased level of cGAS⁺ micronuclei and an increased level of ENPP1 protein expression in a tumor sample (cGAS^{high}ENPP1^{high} tumor) obtained from a subject as compared to that observed in a reference sample, thereby detecting a cGAS^{high}ENPP1^{high} tumor; and administering a therapeutically effective amount of an ENPP1 inhibitor to the subject for whom a cGAS^{high}ENPP1^{high} tumor has been detected.

In one aspect, the disclosure of the present technology provides a method for selecting a subject for the treatment of cancer with an ENPP1 inhibitor, comprising: detecting the presence or absence of an increased ENPP1 protein expression level in a tumor sample obtained from a subject as compared to that observed in a reference sample; and selecting the subject for whom an increased ENPP1 tumor sample has been detected as a subject for the treatment of cancer with an ENPP1 inhibitor.

In one aspect, the disclosure of the present technology provides a method for selecting a subject for the treatment of cancer with an ENPP1 inhibitor comprising: detecting the presence or absence of an increased level of cGAS⁺ micronuclei and a decreased level of ENPP1 protein expression in a tumor sample (cGAS^{high}ENPP1^{high} tumor) obtained from a subject as compared to that observed in a reference sample, thereby detecting a cGAS^{high}ENPP1^{high} tumor; and selecting the subject for whom a cGAS^{high}ENPP1^{high} tumor has been detected as a subject for the treatment of cancer with an ENPP1 inhibitor.

In some embodiments, the reference sample is obtained from a healthy control subject, or normal tissue corresponding to the tumor sample, or contains a predetermined level of ENPP1 protein expression.

In some embodiments, detecting cGAS⁺ micronuclei comprises contacting the tumor sample with an anti-cGAS antibody and measuring the presence of cGAS⁺ micronuclei in the tumor sample.

In some embodiments, detecting ENPP1 protein expression comprises contacting the tumor sample with an anti-ENPP1 antibody and measuring ENPP1 expression levels in the tumor sample.

In some embodiments, the ENPP1 inhibitor is selected from the group consisting of α,β-metADP, α,β-metATP, 2-MeSADP, 2-MeSATP, bzATP, γ-S-α,β-metATP derivatives, ARL 67156, α-borano-β, γ-metATP derivatives, diadenosine boranophosphate derivatives, polyoxometalates [TiW11CoO40]⁸⁻, reactive blue 2 (RB2), quinazoline derivative, suramin, heparin, PPADS, biscoumarin derivative, oxadiazole derivatives, quinazoline derivative, triazole derivative, thioacetamide derivative, isoquinoline derivative, thiadiazolopyrimidinone derivative, STF-1084, thiazolobenzimidazolone derivative, sulfamate derivatives, SR 8314, MV626, MAVU-104, and any combination thereof.

In some embodiments, the methods further comprise administering a therapeutically effective amount of an NT5E inhibitor.

In some embodiments, the NT5E inhibitor is selected from the group consisting of α,β-methylene-ADP, PSB-12379, PSB-12489, AD680, 4-({5-[4-fluoro-1-(2H-indazol-6-yl)-1H-1,2,3-benzotriazol-6-yl]-1H-pyrazol-1-yl}methyl)benzonitrile, 4-({5-[4-chloro-1-(2H-indazol-6-yl)-1H-1,2,3-benzotriazol-6-yl]-1H-pyrazol-1-yl}methyl)benzonitrile, E5NT-02, E5NT-03, 5-fluorouridine-5′-O-[(phosphonomethyl)phosphonic acid], 4-benzoylcytidine-5′-O-[(phosphonomethyl)phosphonic acid], N⁴-[O-(4-benzyloxy)]-3-methyl-cytidine-5′-O-[(phosphonomethyl)phosphonic acid], N⁴-[O-(naphthalen-2-yl-methoxy)]-cytidine-5′-O-[(phosphonomethyl)phosphonic acid], substituted 5′-aminoadenosine derivatives, APCP, 2-trifluoromethyl-4,6-diarylquinolines, benzothiazine compounds, RR2-4, RR6, RR8-9, RR11, RR16, RR18, RR20-21, pyrazolo[3,4-b]pyridines, pyrrolo[2,3-b]pyridines, pyrido[2,3-d]pyrimidines, benzofuro[3,2-b]pyridines, (E)-N′-(1-(3-(4-fluorophenyl)-5-phenyl-4,5-dihydro-1H-pyrazol-1-yl)ethylidene)isonicotinohydrazide, and any combination thereof.

In some embodiments, the combination of an ENPP1 inhibitor and an NT5E inhibitor has a synergistic effect in the treatment of cancer.

In some embodiments, the methods further comprise separately, sequentially, or simultaneously administering radiation therapy, chemotherapy, and/or immunotherapy to the subject.

In some embodiments, the cancer is a solid malignant tumor. In some embodiments, the solid malignant tumor is selected from the group consisting of melanoma, breast cancer, colorectal cancer, lung cancer, prostate cancer, bladder cancer, pancreatic cancer, ovarian cancer, squamous cell carcinoma of the skin, Merkel cell carcinoma, gastric cancer, liver cancer, thyroid cancer, and sarcoma.

In some embodiments, treatment comprises increasing survival, decreasing local tumor recurrence, decreasing metastasis, reducing tumor burden, reducing tumor relapse during post-debulking adjuvant chemotherapy in the subject, reducing the number of cancer cells, reducing the tumor size, eradicating the tumor, inhibiting cancer cell infiltration into peripheral organs, inhibiting or stabilizing tumor growth, and/or stabilizing or improving quality of life in the subject.

In some embodiments, the subject is a mammal. In some embodiments, the mammalian subject is a human.

In one aspect, the disclosure of the present technology provides a method for treating cancer with an immune checkpoint blockade agent in a subject in need thereof, comprising: detecting the presence or absence of a low ENPP1 to cGAS expression ratio in a tumor sample obtained from a subject as compared to that observed in a reference sample; and administering a therapeutically effective amount of one or more immune checkpoint blockade agents to the subject for whom a low ENPP1 to cGAS expression ratio has been detected.

In some embodiments, the reference sample is obtained from a healthy control subject, or normal tissue corresponding to the tumor sample, or contains a predetermined level of ENPP1 protein expression and cGAS⁺ micronuclei.

In some embodiments, detecting ENPP1 protein expression comprises contacting the tumor sample with an anti-ENPP1 antibody and measuring ENPP1 expression levels in the tumor sample, and detecting cGAS⁺ micronuclei comprises contacting the tumor sample with an anti-cGAS antibody and measuring the presence of cGAS⁺ micronuclei in the tumor sample.

In some embodiments, the one or more immune checkpoint blocking agents is selected from the group consisting of anti-PD-L1 antibody, anti-PD-1 antibody, anti-CTLA-4 antibody, ipilimumab, nivolumab, pidilizumab, lambrolizumab, pembrolizumab, atezolizumab, avelumab, durvalumab, MPDL3280A, BMS-936559, MEDI-4736, MSB 00107180, LAG-3, TIM3, B7-H3, B7-H4, TIGIT, AMP-224, MDX-1105, arelumab, tremelimumab, IMP321, MGA271, BMS-986016, lirilumab, urelumab, PF-05082566, IPH2101, MEDI-6469, CP-870,893, Mogamulizumab, Varlilumab, Galiximab, AMP-514, AUNP 12, Indoximod, NLG-919, INCB024360, CD80, CD86, ICOS, DLBCL inhibitors, BTLA, PDR001, and any combination thereof.

In some embodiments, the methods further comprise separately, sequentially, or simultaneously administering radiation therapy and/or chemotherapy to the subject.

In some embodiments, the cancer is a solid malignant tumor. In some embodiments, the solid malignant tumor is selected from the group consisting of melanoma, breast cancer, colorectal cancer, lung cancer, prostate cancer, bladder cancer, pancreatic cancer, ovarian cancer, squamous cell carcinoma of the skin, Merkel cell carcinoma, gastric cancer, liver cancer, thyroid cancer, and sarcoma.

In some embodiments, treatment comprises increasing survival, decreasing local tumor recurrence, decreasing metastasis, reducing tumor burden, reducing tumor relapse during post-debulking adjuvant chemotherapy in the subject, reducing the number of cancer cells, reducing the tumor size, eradicating the tumor, inhibiting cancer cell infiltration into peripheral organs, inhibiting or stabilizing tumor growth, and/or stabilizing or improving quality of life in the subject.

In some embodiments, the subject is a mammal. In some embodiments, the mammalian subject is a human.

In one aspect, the disclosure of the present technology provides a method for treating cancer in a subject in need thereof, comprising: administering a therapeutically effective amount of an ENPP1 inhibitor and an NT5E inhibitor to the subject.

In some embodiments, the ENPP1 inhibitor is selected from the group consisting of α,β-metADP, α,β-metATP, 2-MeSADP, 2-MeSATP, bzATP, 7-S-α,β-metATP derivatives, ARL 67156, α-borano-β, 7-metATP derivatives, diadenosine boranophosphate derivatives, polyoxometalates [TiW11CoO40]⁸⁻, reactive blue 2 (RB2), quinazoline derivative, suramin, heparin, PPADS, biscoumarin derivative, oxadiazole derivatives, quinazoline derivative, triazole derivative, thioacetamide derivative, isoquinoline derivative, thiadiazolopyrimidinone derivative, STF-1084, thiazolobenzimidazolone derivative, sulfamate derivatives, SR 8314, MV626, MAVU-104, and any combination thereof.

In some embodiments, the NT5E inhibitor is selected from the group consisting of α,β-methylene-ADP, PSB-12379, PSB-12489, AD680, 4-({5-[4-fluoro-1-(2H-indazol-6-yl)-1H-1,2,3-benzotriazol-6-yl]-1H-pyrazol-1-yl}methyl)benzonitrile, 4-({5-[4-chloro-1-(2H-indazol-6-yl)-1H-1,2,3-benzotriazol-6-yl]-1H-pyrazol-1-yl}methyl)benzonitrile, E5NT-02, E5NT-03, 5-fluorouridine-5′-O-[(phosphonomethyl)phosphonic acid], 4-benzoylcytidine-5′-O-[(phosphonomethyl)phosphonic acid], N⁴-[O-(4-benzyloxy)]-3-methyl-cytidine-5′-O-[(phosphonomethyl)phosphonic acid], N⁴-[O-(naphthalen-2-yl-methoxy)]-cytidine-5′-O-[(phosphonomethyl)phosphonic acid], substituted 5′-aminoadenosine derivatives, APCP, 2-trifluoromethyl-4,6-diarylquinolines, benzothiazine compounds, RR2-4, RR6, RR8-9, RR11, RR16, RR18, RR20-21, pyrazolo[3,4-b]pyridines, pyrrolo[2,3-b]pyridines, pyrido[2,3-d]pyrimidines, benzofuro[3,2-b]pyridines, (E)-N′-(1-(3-(4-fluorophenyl)-5-phenyl-4,5-dihydro-1H-pyrazol-1-yl)ethylidene)isonicotinohydrazide, and any combination thereof.

In some embodiments, the combination of an ENPP1 inhibitor and an NT5E inhibitor has a synergistic effect in the treatment of cancer.

In some embodiments, the methods further comprise separately, sequentially, or simultaneously administering radiation therapy, chemotherapy, immunotherapy, and/or therapies that induce DNA damage or genomic instability to the subject.

In some embodiments, the methods further comprise separately, sequentially, or simultaneously administering to the subject one or more immune checkpoint blocking agents selected from the group consisting of anti-PD-L1 antibody, anti-PD-1 antibody, anti-CTLA-4 antibody, ipilimumab, nivolumab, pidilizumab, lambrolizumab, pembrolizumab, atezolizumab, avelumab, durvalumab, MPDL3280A, BMS-936559, MEDI-4736, MSB 00107180, LAG-3, TIM3, B7-H3, B7-H4, TIGIT, AMP-224, MDX-1105, arelumab, tremelimumab, IMP321, MGA271, BMS-986016, lirilumab, urelumab, PF-05082566, IPH2101, MEDI-6469, CP-870,893, Mogamulizumab, Varlilumab, Galiximab, AMP-514, AUNP 12, Indoximod, NLG-919, INCB024360, CD80, CD86, ICOS, DLBCL inhibitors, BTLA, PDR001, and any combination thereof.

In some embodiments, the cancer is a solid malignant tumor. In some embodiments, the solid malignant tumor is selected from the group consisting of melanoma, breast cancer, colorectal cancer, lung cancer, prostate cancer, bladder cancer, pancreatic cancer, ovarian cancer, squamous cell carcinoma of the skin, Merkel cell carcinoma, gastric cancer, liver cancer, thyroid cancer, and sarcoma.

In some embodiments, treatment comprises reducing tumor volume, increasing immune infiltration, decreasing metastasis, treating primary tumors, increasing immune activation against tumors, sensitizing the tumor to immunotherapy, sensitizing the tumor to radiation therapy, sensitizing the tumor to chemotherapy, sensitizing the tumor to therapies that induce DNA damage or genomic instability, increasing survival, decreasing local tumor recurrence, reducing tumor burden, reducing tumor relapse during post-debulking adjuvant chemotherapy in the subject, reducing the number of cancer cells, reducing the tumor size, eradicating the tumor, inhibiting cancer cell infiltration into peripheral organs, inhibiting or stabilizing tumor growth, and/or stabilizing or improving quality of life in the subject.

In some embodiments, the subject is a mammal. In some embodiments, the mammalian subject is a human.

In one aspect, the disclosure of the present technology provides a method for treating cancer in a subject in need thereof, comprising: administering a therapeutically effective amount of a STING agonist and an NT5E inhibitor to the subject.

In some embodiments, the STING agonist is selected from the group consisting of c-di-AMP, c-di-GMP, diABZIs, 3′3′-cGAMP, 2′3′-cGAMP, 5,6-dimethylxanthenone-4-acetic acid (DMXAA), macrocycle-bridged STING agonist E7766, GSK3745417, MK-1454, MK-2118, ADU-S100, SB11285, BMS-98630, and any combination thereof.

In some embodiments, the NT5E inhibitor is selected from the group consisting of α,β-methylene-ADP, PSB-12379, PSB-12489, AD680, 4-({5-[4-fluoro-1-(2H-indazol-6-yl)-1H-1,2,3-benzotriazol-6-yl]-1H-pyrazol-1-yl}methyl)benzonitrile, 4-({5-[4-chloro-1-(2H-indazol-6-yl)-1H-1,2,3-benzotriazol-6-yl]-1H-pyrazol-1-yl}methyl)benzonitrile, E5NT-02, E5NT-03, 5-fluorouridine-5′-O-[(phosphonomethyl)phosphonic acid], 4-benzoylcytidine-5′-O-[(phosphonomethyl)phosphonic acid], N⁴-[O-(4-benzyloxy)]-3-methyl-cytidine-5′-O-[(phosphonomethyl)phosphonic acid], N⁴-[O-(naphthalen-2-yl-methoxy)]-cytidine-5′-O-[(phosphonomethyl)phosphonic acid], substituted 5′-aminoadenosine derivatives, APCP, 2-trifluoromethyl-4,6-diarylquinolines, benzothiazine compounds, RR2-4, RR6, RR8-9, RR11, RR16, RR18, RR20-21, pyrazolo[3,4-b]pyridines, pyrrolo[2,3-b]pyridines, pyrido[2,3-d]pyrimidines, benzofuro[3,2-b]pyridines, (E)-N′-(1-(3-(4-fluorophenyl)-5-phenyl-4,5-dihydro-1H-pyrazol-1-yl)ethylidene)isonicotinohydrazide, and any combination thereof.

In some embodiments, the combination of STING agonist and an NT5E inhibitor has a synergistic effect in the treatment of cancer.

In some embodiments, the methods further comprise separately, sequentially, or simultaneously administering radiation therapy, chemotherapy, immunotherapy, and/or therapies that induce DNA damage or genomic instability to the subject.

In some embodiments, the methods further comprise separately, sequentially, or simultaneously administering to the subject one or more immune checkpoint blocking agents selected from the group consisting of anti-PD-L1 antibody, anti-PD-1 antibody, anti-CTLA-4 antibody, ipilimumab, nivolumab, pidilizumab, lambrolizumab, pembrolizumab, atezolizumab, avelumab, durvalumab, MPDL3280A, BMS-936559, MEDI-4736, MSB 00107180, LAG-3, TIM3, B7-H3, B7-H4, TIGIT, AMP-224, MDX-1105, arelumab, tremelimumab, IMP321, MGA271, BMS-986016, lirilumab, urelumab, PF-05082566, IPH2101, MEDI-6469, CP-870,893, Mogamulizumab, Varlilumab, Galiximab, AMP-514, AUNP 12, Indoximod, NLG-919, INCB024360, CD80, CD86, ICOS, DLBCL inhibitors, BTLA, PDR001, and any combination thereof.

In some embodiments, the cancer is a solid malignant tumor. In some embodiments, the solid malignant tumor is selected from the group consisting of melanoma, breast cancer, colorectal cancer, lung cancer, prostate cancer, bladder cancer, pancreatic cancer, ovarian cancer, squamous cell carcinoma of the skin, Merkel cell carcinoma, gastric cancer, liver cancer, thyroid cancer, and sarcoma.

In some embodiments, treatment comprises reducing tumor volume, increasing immune infiltration, decreasing metastasis, treating primary tumors, increasing immune activation against tumors, sensitizing the tumor to immunotherapy, sensitizing the tumor to radiation therapy, sensitizing the tumor to chemotherapy, sensitizing the tumor to therapies that induce DNA damage or genomic instability, increasing survival, decreasing local tumor recurrence, reducing tumor burden, reducing tumor relapse during post-debulking adjuvant chemotherapy in the subject, reducing the number of cancer cells, reducing the tumor size, eradicating the tumor, inhibiting cancer cell infiltration into peripheral organs, inhibiting or stabilizing tumor growth, and/or stabilizing or improving quality of life in the subject.

In some embodiments, measuring the presence of cGAS⁺ micronuclei in the tumor sample comprises one or more of: quantifying the number of cGAS⁺ micronuclei in a defined high-power field; performing a semi-quantitative assessment; and measuring cGAS⁺ micronuclei as a fraction of cGAS⁺ micronuclei/primary nuclei.

### BRIEF DESCRIPTION OF THE DRAWINGS

FIG. 1A shows representative images of 4T1 triple-negative breast cancer (TNBC) cells undergoing anaphase with various chromosome segregation defects stained using DAPI (DNA) and anti-centromere protein. Scale bar: 5 μm.

FIG. 1B shows frequency of anaphase cells with chromosome segregation defects in poorly metastatic (B16F0 and B16F1) and highly metastatic (B16F10, CT26, and 4T1) cells. Bars represent average±SD, n=150 cells, 3 experiments, **** p<0.0001, two-sided t-test.

FIG. 1C shows a representative image of a 4T1 cell with a micronucleus stained using DAPI and anti-cGAS antibody. Scale bar: 5 μm.

FIG. 1D shows percentage of micronuclei in the various cell lines. Bars represent median, n=5-17, high-power fields comprising 1820-5304 cells per condition, **** p<0.0001, two-sided Mann-Whitney test.

FIG. 1E shows cGAMP levels in cell lysates. Bars represent median values, n=6-12 independent experiments, * p<0.05, **p<0.01, two-sided Mann-Whitney test.

FIG. 1F shows cGAMP levels in tumor lysates. Bars represent median values, n=10-35 tumor samples, **p<0.01, **** p<0.0001, two-sided Mann-Whitney test.

FIG. 1G shows relative expression levels of Ifnb1 and ISGs in mock, cGAMP, or Poly(I:C)-transfected 4T1 cells. Bars represent mean±SD, n=3 independent experiments (n=2 for Poly(I:C)-treated) each with two technical replicates. Significance tested using 2-way ANOVA.

FIG. 1H shows normalized STING protein levels in control, Cgas-KO, and STING-KO cells. Bars represent mean±SD, n=3 independent experiments, * p<0.05, ratio-paired t-test.

FIG. 1I shows relative expression levels of Ifnb1 and ISGs in mock or cGAMP-transfected Cgas-KO 4T1 cells in the presence or absence of the STING inhibitor C-176. Bars represent mean±SD, n=3 independent experiments each with two technical replicates. Significance was tested using 2-way ANOVA.

FIG. 2A shows the number of surface lung metastasis in mice transplanted with primary 4T1 tumors with low (L, <median) or high (H, >median) cGAMP levels at resection. Data points represent mean±SD, two-sided paired t-test, n=2 independent experiments, 18 mice.

FIG. 2B shows cGAMP levels in primary and matched-recurrent tumor lysates. Two-sided ratio-paired-t-test.

FIG. 2C shows representative hematoxylin and eosin-stained lungs 3 weeks after resection of control or STING-depleted orthotopically transplanted 4T1 tumors. Scale bar: 4 mm.

FIG. 2D shows number of surface lung metastasis arising from orthotopically transplanted and subsequently resected 4T1 primary tumors. Bars represent median values, *p<0.05, ****p<0.0001, two-sided Mann-Whitney test, n=14-25 animals per condition.

FIG. 2E shows number of surface lung metastasis arising from direct tail vein inoculation of CT26 cells. Bars represent median values, *p<0.05, ****p<0.0001, two-sided Mann-Whitney test, n=14-25 animals per condition.

FIG. 2F shows number of surface lung metastasis arising from direct tail vein inoculation of B16F10 cells. Bars represent median values, *p<0.05, ****p<0.0001, two-sided Mann-Whitney test, n=14-25 animals per condition.

FIG. 2G shows representative lung images from animals tail-vein-injected with control or STING-KO B16F10 cells whereby dark (black) melanoma metastases can be seen primarily in the lungs of control-injected animals.

FIG. 3A shows representative images of vehicle and C-176 treated B16F10 cells at 0 and 12 hours after wound creation.

FIG. 3B shows relative wound area (left) and normalized invasion (right) upon treatment of B16F10, 4T1, and CT26 cells with C-176 or vehicle control. Bars represent mean±SD, *p<0.05, **p<0.01, ***p<0.001, two-sided t-test.

FIG. 3C demonstrates gene-set enrichment analysis (GSEA) results showing HALLMARK gene sets that are differentially enriched between control and STING-KO B16F10 cells. Significance was tested using one-sided weighted Smirnov-Kolmogorov test corrected for multiple tests.

FIG. 3D demonstrates gene-set enrichment analysis (GSEA) results showing HALLMARK gene sets that are differentially enriched between vehicle and C-176-treated B16F10 cells. Significance was tested using one-sided weighted Smirnov-Kolmogorov test corrected for multiple tests.

FIG. 3E shows a heat map of 724 genes that are differentially expressed between vehicle and C-176 treated control but not STING-KO B16F10 cells identifying two gene modules.

FIG. 3F shows pathway enrichments in the differentially expressed gene modules upon C-176 treatment.

FIG. 4A shows animal survival over time upon tail vein inoculation of CT26 cells in BALB/c mice that were treated with C-176 or a corresponding vehicle control. Significance was tested using log-rank test, n=15 animals per experimental arm.

FIG. 4B shows animal survival over time upon tail vein inoculation of B16F10 cells in C57BL/6 mice that were treated with C-176 or a corresponding vehicle control. Significance was tested using log-rank test, n=15 animals per experimental arm.

FIG. 4C shows animal survival over time upon tail vein inoculation of 4T1 cells in BALB/c mice that were treated with C-176 or a corresponding vehicle control. Significance was tested using log-rank test, n=15 animals per experimental arm.

FIG. 4D shows animal survival over time upon tail vein inoculation of CT26 cells in BALBc mice that were treated with H151 or a corresponding vehicle control. Significance tested using log-rank test, n=15 animals per experimental arm.

FIG. 5A shows a representative high-resolution image of human mucosal melanoma sample stained with DAPI (DNA) and anti-cGAS antibody showing selective localization of cGAS at micronuclei. Scale bar: 5 μm.

FIG. 5B shows scatter plots depicting the relationship between the number of cGAS+ micronuclei per high-power field in mucosal melanoma samples and the fraction of the genome altered derived from low pass whole-genome sequencing (top) or tumor mutational burden as defined as the number of mutations per mega-base (bottom) from the MSK-IMPCT test.

FIG. 5C shows a bar graph depicting the relationship between tumor cGAS and STING protein levels in TNBC tumors. Significance was tested using x²-test, n=179 tumors.

FIG. 5D shows images from the same TNBC tumor in FIG. 5C stained using DAPI (DNA), anti-cGAS, and anti-STING antibodies, illustrating the inverse correlation between the frequency of cGAS+ micronuclei and cancer cell-intrinsic STING staining. Scale bar: 50 μm.

FIG. 5E shows distant metastasis-free survival (DMFS) of patients over time with TNBC stratified based on tumor cGAS and STING staining intensity. Significance was tested using log-rank test, n=159 (upper) and 155 (bottom) patients.

FIG. 6A shows representative images of B16F10 and CT26 cells with micronuclei stained using DAPI (DNA) and anti-cGAS antibody. Scale bar: 5 μm.

FIG. 6B shows immunoblots of control, Cgas-KO, and STING-KO B16F10, 4T1, and CT26 cells stained for cGAS, STING. β-actin was used as a loading control.

FIG. 6C shows immunoblots of control, cGAS-depleted, and STING-depleted 4T1 cells stained for cGAS, STING. β-actin was used as a loading control.

FIG. 6D shows interferon-β levels in tumor lysates in control, Cgas-KO, and STING-KO 4T1 tumors. An immunotherapy treated pancreatic tumor after anti-PD1 treatment was used as a reference positive control. Bars represent mean±SD, n=14-15 tumors per conditions (4T1) and n=8 tumors for the pancreatic tumors, **** p<0.0001.

FIG. 7A shows tumor volume of resected orthotopically transplanted primary 4T1 tumors with low (L, <median) or high (H, >median) cGAMP levels. Bars represent median values, n=28 tumors.

FIG. 7B shows an experimental schema for metastasis experiments shown in FIG. 2 and FIG. 7C-7D.

FIG. 7C shows tumor volume of resected orthotopically transplanted control, Cgas-KO and STING-KO primary 4T1 tumors. Bars represent median values, **p<0.01, two-sided Mann-Whitney test, n=14-15 tumors per condition.

FIG. 7D shows tumor volume of resected orthotopically transplanted control and STING-depleted primary 4T1 tumors. Bars represent median values, *p<0.05, two-sided Mann-Whitney test, n=8 (control) and 16 (STING-KD) animals.

FIG. 7E shows the number of surface lung metastases of STING-depleted 4T1 tumors in animals arising after tumor resection. Bars represent median values, *p<0.05, two-sided Mann-Whitney test, n=8 (control) and 16 (STING-KD) animals.

FIG. 8A shows immunoblots of vehicle and C-176 treated B16F10, 4T1, and CT26 cells stained for total and phosphorylated forms of p65 and RelB with cyclophilin B as a loading control.

FIG. 8B shows relative amounts of p-RelB and p-p65 normalized to total RelB and p65, respectively, in cells treated with vehicle or C-176.

FIG. 8C shows immunoblots of vehicle and C-176 treated B16F10, 4T1, and CT26 cells transfected with cGAMP and treated with increasing doses of C-176, stained for total and phosphory-lated forms of IRF3 with cyclophilin B as a loading control.

FIG. 8D shows normalized counts of vehicle and C-176 treated B16F10, 4T1, and CT26 cells at various time points after treatment initiation.

FIG. 9A shows expression levels of three ISGs in control and STING-KO B16F10 cells treated with C-176 or vehicle control. ** p<0.01, *** p<0.001, **** p<0.0001, n=3 biological replicates.

FIG. 9B shows (Top) Log 2-fold change in the expression of STING-dependent genes comparing vehicle and C-176 treatment of control and STING-KO B16F10 cells; (Bottom) Log 2-fold change in the expression of C-176-dependent genes comparing control and STING-KO B16F10 cells that are either treated with C-176 or vehicle control. Significance was tested with two-sided paired-t-test.

FIG. 9C shows enrichment plots showing three HALLMARK gene sets that are differentially expressed between control and STING-KO (left) as well as vehicle and C-176-treated (right) B16F10 cells.

FIG. 9D shows surface lung metastases after tail vein inoculation of CT26 cells. Bars represent median values, * p<0.05, two-sided Mann-Whitney test.

FIG. 9E shows survival of animals inoculated with STING-KO B16F10 via tail-vein injection and treated with C-176 or vehicle control. Significance was tested using the log-rank test.

FIG. 9F shows tumor volume of resected orthotopically transplanted primary 4T1 tumors after treated with C-176 or vehicle control.

FIG. 10A shows representative images of MDA-MB-231 TNBC cell pellets stained with DAPI (DNA) and three independent anti-human-cGAS antibodies. White arrows denote cGAS staining of micronuclei.

FIG. 10B shows immunoblots of control and cGAS-depleted MDA-MB-231 cell lysates stained for cGAS. β-actin was used as a loading control.

FIG. 10C shows representative images of human mucosal melanoma samples stained with DAPI (DNA) and anti-human-cGAS antibody (LS-C757990) illustrating tumors with few (left) and numerous (right) cGAS+ micronuclei. Scale bar: 50 μm.

FIG. 10D shows distant metastasis-free survival (DMFS) of patients with TNBC stratified based on tumor cGAS and STING staining intensity. Significance tested using log-rank test, n=155 patients.

FIG. 11A shows representative immunofluorescence images of control and ENPP1-depleted MDA-MB-231 CIN^{high} cells stained with DAPI (DNA) and anti-ENPP1 antibody. Scale bar: 50 μm.

FIG. 11B shows immunohistochemistry of an orthotopically transplanted MBA-MB-231 tumor using anti-ENPP1 antibody.

FIG. 11C shows ratio of extracellular-to-intracellular cGAMPs in 4T1 cells. Bars represent median, n=10 independent experiments, ** p<0.01, two-sided Mann-Whitney test.

FIG. 11D shows overall survival rate over time of animals that were orthotopically transplanted by control and Enpp1-knockout 4T1 tumors followed by tumor resection 7 days later. n=15 animals per condition, significance tested using log-rank test.

FIG. 11E shows (Left) quantification of surface lung metastases after tail vein injection of control and Enpp1-knockout 4T1 cells, bars represent median, n=13-15 animals per condition, ****p<0.0001, two-sided Mann-Whitney test; and (Right) representative hematoxylin and eosin-stained lungs from animals injected with control and ENPP1-knockout 4T1 cells, Scale bar: 3 mm.

FIG. 12A shows a schematic of the generation of adenosine from extracellular cGAMP hydrolysis.

FIG. 12B shows the effects of extracellular adenosine on cancer and immune cells.

FIG. 12C shows normalized adenosine concentration (per 10⁷ cells after 16 hours incubation in serum-free media) in conditioned media of control, Cgas-KO, Enpp1-KO 4T1 cells. Bars represent mean±s.e.m., n=4 independent experiments, *p<0.05, two-sided t-test.

FIG. 12D shows percent wound remaining after 24 hours in control, Cgas-KO, and Enpp1-KO 4T1 cells treated with cGAMP or cGAMP and the adenosine receptor blocker, PSB115.

FIG. 12E shows representative immunohistochemistry (IHC) of control and ENPP1-knockout triple-negative breast cancer (TNBC) lung metastases stained using an anti-CD45 antibody.

FIG. 12F shows (left) the number of metastasis-infiltrating CD8+ T-cells and (right) representative IHC of control ENPP1-knockout TNBC lung metastases stained using anti-CD8 antibody. Bars represent median, n=13-31 metastases, **** p<0.0001, two-sided Mann-Whitney test.

FIG. 12G shows percentage of CD45+, CD11b+Ly6G+, CD4+, and CD8+ cells out of the total cells as well as the percentage of PD1+ cells out of the CD3+CD4+ and CD3+CD8+ cells obtained from dissociated lungs after injection with control or ENPP1-knockout 4T1 cells. n=5 animals per group.

FIG. 12H shows GM-CSF levels measured in orthotopically transplanted control and ENPP1-knockout tumors. Bars represent median, n=15 tumors per condition, ** p<0.01, two-sided Mann-Whitney test.

FIG. 13A shows relative ENPP1 mRNA levels in 4T1 and CT26 cells. **** p<0.0001, two-tailed t-test.

FIG. 13B shows a schematic diagram of immunotherapy experiments.

FIG. 13C shows growth curves of control and Enpp1-KO orthotopically transplanted tumors 4T1 upon treatment with combined ICB or corresponding isotype controls. Data points represent mean±s.e.m., n=15 animals per group, ****p<0.0001, two-sided t-test.

FIG. 13D shows survival over time of animals after orthotopic transplantation with control and Enpp1-KO 4T1 cells treated with combined ICB or corresponding isotype controls. Significance tested using log-rank test, *** p<0.001, n=15 animals per group.

FIG. 13E shows surface lung metastases after tail vein injection of eGFP and eGFP-ENPP1-expressing CT26 cells. Bars represent median, n=15 animals per condition, **** p<0.0001, two-sided Mann-Whitney test.

FIG. 13F shows survival over time of BALB/c mice injected with eGFP or eGFP-ENPP1 expressing CT26 cells, treated with combined ICB or isotype controls. n=15 animals per group, significance tested using log-rank test, ***p<0.001.

FIG. 14A shows representative images of human TNBCs stained using anti-ENPP1 antibody. Scale bar: 100 μm.

FIG. 14B shows distant-metastasis-free survival in patients with TNBC stratified based on their ENPP1 expression. n=69, significance tested using log-rank test.

FIG. 14C shows percentage of tumor-infiltrating lymphocytes (TILs) in breast tumors stratified based on their ENPP1 expression.

FIG. 14D shows representative images of human breast cancers stained using anti-ENPP1 or anti-CD8 antibodies. Scale bar: 100 μm.

FIG. 14E shows percent objective response rate (ORR) to anti-PD1/PD-L1 therapy as a function of ENPP1 expression by cancer type for tumor histologies with high levels of CGAS expression.

FIG. 14F shows a schematic illustrating the consequence of ENPP1 activity (right) or its absence (left) on cancer metastasis and immune evasion.

FIG. 15A shows representative images of 4T1 cells undergoing error-free anaphase or anaphase with evidence of chromosome missegregation. Scale bar: 2 μm.

FIG. 15B shows representative image of a 4T1 cells with micronuclei stained using DAPI and anti-cGAS antibody. Scale bar: 2 μm.

FIG. 15C shows immunoblots of control, cGAS-knockout, and STING-knockout 4T1 cell lysates stained using anti-STING, anti-cGAS, α-tubulin and β-actin antibodies.

FIG. 15D shows cGAMP levels in cell lysates of 4T1 cells incubated in serum-free media for 24 hour. cAGMP levels were normalized for cell number.

FIG. 15E shows relative intracellular and extracellular cGAMP production in 4T1 cells. Bars represent mean±s.e.m. n=6 independent experiments ** p<0.01, two-sided t-test.

FIG. 16A shows volcano plot showing differentially expressed genes between MDA-MB-231 cells expressing MCAK or Kif2b (CIN_{low}) or dominant-negative MCAK (CIN^{high}).

FIG. 16B shows immunoblots of CIN_{low} and CIN^{high} cell lysates stained with anti-ENPP1 and anti-3-actin antibodies.

FIG. 16C shows representative immunohistochemistry (IHC) images of control and ENPP1-depleted orthotopically transplanted human TNBCs stained using anti-ENPP1 antibody. Scale bar: 200 μm.

FIG. 16D shows immunoblots of control and ENPP1-depleted CIN^{high} MDA-MB-231 cell lysates stained using anti-ENPP1 and anti-β-actin antibody.

FIG. 16E shows ENPP1 mRNA levels in 4T1 cells as well as cells derived from lung metastases. ***p<0.001, two-tailed t-test.

FIG. 16F shows sequences of 4T1 single-cell derived clones showing successful ENPP1 knockout and absence of wildtype allele.

FIG. 16G shows proliferation of control and Enpp1-knockout 4T1 cells over time.

FIG. 16H shows volume of orthotopically transplanted control and ENPP1-knockout tumors over time. Data points represent average s.e.m.

FIG. 16I shows recurrent primary tumor weight after resection of control or Enpp1-knockout primary tumor resection, bars represent median, * p<0.05, ** p<0.01, two-sided Mann-Whitney test.

FIG. 16J shows surface lung metastases after resection of control or Enpp1-knockout primary tumor resection. Bars represent median, * p<0.05, ** p<0.01, two-sided Mann-Whitney test.

FIG. 16K shows representative bioluminescence images of BALB/c mice 35 days after orthotopic transplantation with control and Enpp1-KO 4T1 tumors followed by tumor resection on day 7.

FIG. 16L shows overall survival of animals injected by control or Enpp1 knockout 4T1 cells. n=15 animals per condition, significance tested using log-rank test.

FIG. 17A shows a schematic of extracellular adenosine metabolism illustrating an indirect fluorescence-based method of quantifying extracellular adenosine production. By subtracting fluorescence measurements obtained from media containing adenosine deaminase inhibitor, PSB115, from media without the inhibitor, one is able to quantify the amounts of downstream products arising from extracellular adenosine degradation.

FIG. 17B shows relative fluorescence intensity at 600 nm with and without the addition of PSB115 in the presence of increasing amounts of exogenous cGAMP.

FIG. 18A shows semi-quantitative measurement of tumor necrosis in control and ENPP1-depleted human TNBC xenografts.

FIG. 18B shows representative IHC images of control and ENPP1-depleted TNBC xenografts stained using NK1.1 (to stain NK-cells), Scale bar: 200 μm.

FIG. 18C shows FACS gating scheme for experiments shown in FIG. 12G.

FIG. 19A shows immunoblots of control and luciferase expressing wildtype or Enpp1-KO 4T1 cells stained using anti-tdTomato-Luciferase and Lamin B1 antibodies.

FIG. 19B shows spider plots showing growth of orthotopically transplanted control and ENPP1-KO 4T1 tumors treated with combined ICB or isotype control antibodies.

FIG. 19C shows representative immunofluorescence images of control, eGFP-expressing, and eGFP-ENPP1 expressing CT26 cells stained using DAPI (DNA). Scale bar: 10 μm.

FIG. 20A shows ENPP1 mRNA levels across human cancer types found in the TCGA database.

FIG. 20B shows hazard ratio for death of patients stratified by tumor ENPP1 median expression values. Data points represent HR±95% CI. Red data points represent p<0.05.

FIG. 20C shows CGAS and ENPP1 mRNA expression levels across breast cancer subtypes found in the TCGA. Bars represent median±interquartile range, ** p<0.01,* p<0.0001, two-sided Mann-Whitney test.

FIG. 20D shows overall survival of breast cancer patients stratified by tumor receptor status and ENPP1 expression levels. Significance tested using log-rank test.

FIG. 21A shows ENPP1 mRNA expression levels across human tumor-derived organoids. Bars represent median values, * p<0.05, two-sided t-test.

FIG. 21B shows percentage of mucosal melanoma patients with tumor-specific or stromal specific ENPP1 staining patterns in primary as well as metastatic mucosal melanoma human tumor samples. *p<0.05, Z-test.

FIG. 21C shows representative immunofluorescence images of low magnification images of lymph node metastases from mucosal melanoma stained using DAPI (DNA) and anti-ENPP1 antibody showing selective membrane staining of ENPP1 on metastatic cancer cells. Scale bar: 1 mm.

FIG. 21D shows representative immunofluorescence images of high magnification images of lymph node metastases from mucosal melanoma stained using DAPI (DNA) and anti-ENPP1 antibody showing selective membrane staining of ENPP1 on metastatic cancer cells. Scale bar: 50 μm.

FIG. 21E shows distribution tumor samples exhibiting stroma-specific and cancer cell-specific staining patterns of ENPP1 in three independent cohorts of human breast cancer.

FIG. 21F shows overall survival (OS) in patients with TNBC stratified based on their ENPP1 expression. n=73, significance tested using log-rank test.

FIG. 21G shows relapse-free survival (RFS) in patients with ER+ breast cancer stratified based on their ENPP1 expression. n=78 patients, significance tested using log-rank test.

FIG. 22A shows percentage of tumor or stromal CD8+ T-cells an independent human breast cancer cohort (Cohort 2) stratified based on their tumor and stromal ENPP1 expression.

FIG. 22B shows percentage of tumor or stromal CD8+ T-cells an independent human breast cancer cohort (Cohort 3) stratified based on their tumor and stromal ENPP1 expression.

FIG. 22C shows tumor immune infiltration inferred using the CIBERSORT method on breast tumors found in the TCGA. Box plots represent median, lower and upper quartiles; error bars represent 10^{th} and 90^{th} percentiles. n=1079 tumors, ****p<0.0001, two-sided Mann-Whitney test.

FIG. 22D shows gene-set enrichment plots comparing cGAS^{high}-ENPP1-high and cGAS-^{high}ENPP1-^{low} human breast tumors showing upregulation of inflammation related gene sets in ENPP1-low tumors.

FIG. 22E shows correlation between cytotoxic lymphocyte score and either ENPP1 levels or the ratio of ENPP1-to-cGAS mRNA levels in 3 independent sarcoma datasets.

FIG. 23A shows representative immunofluorescence images of mucosal melanoma samples stained for using DAPI (DNA), anti-cGAS antibody, and anti-ENPP1 antibody. Scale bar: 100 μm.

FIG. 23B shows a representative high-resolution immunofluorescence image of a mucosal melanoma sample stained using DAPI (DNA) or anti-cGAS antibody showing cGAS localization to micronuclei. Scale bar: 2 μm.

FIG. 23C shows (Left) Representative multispectral immunofluorescence images of mucosal melanoma samples stained using DAPI (DNA), anti-CD8, and anti-Melan A antibodies; and (Right) CD8+ T-cell density as a function of combined cGAS and ENPP1 staining intensity in mucosal melanoma samples. Scale bar: 100 μm. Bars represent median, *p<0.05 two-sided Mann-Whitney test.

FIG. 23D shows percent objective response rate (ORR) to anti-PD1/PD-L1 therapy by cancer type in tumor histologies with low levels of CGAS expression.

FIG. 23E shows ENPP1 and cGAS mRNA expression levels of bladder tumors stratified by response to ICB. Bars represent median±interquartile range, * p<0.05, **** p<0.0001, two-sided Mann-Whitney test.

FIG. 24 shows tumor volume over time of orthotopically transplanted wildtype, ENPP1 knockout, NT5E knockout, or ENPP1/NT5E double knockout 4T1 triple negative breast tumors showing reduced tumor growth in the ENPP1 and NT5E double knockout.

FIG. 25A is a schematic illustrating the high-throughput quantification of CIN in human cancer process of data analysis and collection used in a high-grade serous ovarian cancer cohort (n=43 patients). Each sample was either dissociated into single cells and then subjected to single-cell RNA sequencing, single-cell DNA sequencing using the DLP platform, or subjected to standard histologic processing and embedding.

FIGS. 25B and 25C are images of immunofluorescence staining of high-grade serous ovarian cancer samples stained with DAPI for DNA and anti-cGAS antibody showing punctate staining of cGAS at micronuclei. FIG. 25B is an example showing low frequency of micronuclei (fewer puncta), whereas FIG. 25C is an example of a sample with high levels of micronuclei (more red puncta).

FIG. 25D is a histogram showing the distribution of the fraction of micronuclei/primary nuclei for the full high-grade serous ovarian cancer cohort of 100 patients.

FIG. 25E is an image showing an example of a high-grade serous ovarian cancer sample stained with DAPI for DNA and anti-cGAS antibody.

FIG. 25F is a single cell DNA copy number tracing from single cell DNA sequencing.

FIG. 25G is a copy number tracing obtained from single-cell RNA sequence data.

FIG. 25H is a genomic copy number profile obtained from bulk whole genome sequence data.

FIG. 25I is a chart showing the correlation between the frequency of cGAS⁺ micronuclei and the mean pairwise distance, which is a metric that infers copy number heterogeneity inferred from scRNA-seq data.

FIG. 26 is a chart showing tumor volume over time (days) of wild-type or cGAS-knockout (cGAS KO) 4T1 tumors that were orthotopically transplanted in the mammary fat pad of mice then treated with PBS (vehicle) or ADU-S100 (a STING agonist).

### DETAILED DESCRIPTION

It is to be appreciated that certain aspects, modes, embodiments, variations and features of the present technology are described below in various levels of detail in order to provide a substantial understanding of the present technology.

In practicing the present methods, many conventional techniques in molecular biology, protein biochemistry, cell biology, immunology, microbiology and recombinant DNA are used. See, e.g., Sambrook and Russell eds. (2001) Molecular Cloning: A Laboratory Manual, 3rd edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology. Methods to detect and measure levels of polypeptide gene expression products (i.e., gene translation level) are well-known in the art and include the use of polypeptide detection methods such as antibody detection and quantification techniques. (See also, Strachan & Read, Human Molecular Genetics, Second Edition. (John Wiley and Sons, Inc., NY, 1999)).

### I. DEFINITIONS

The definitions of certain terms as used in this specification are provided below. Unless defined otherwise, all technical and scientific terms used herein generally have the same meaning as commonly understood by one of ordinary skill in the art to which this present technology belongs.

As used in this specification and the appended claims, the singular forms “a,” “an,” and “the” include plural referents unless the content clearly dictates otherwise. For example, reference to “a cell” includes a combination of two or more cells, and the like. Generally, the nomenclature used herein and the laboratory procedures in cell culture, molecular genetics, organic chemistry, analytical chemistry and nucleic acid chemistry and hybridization described below are those well-known and commonly employed in the art.

As used herein, the term “about” in reference to a number is generally taken to include numbers that fall within a range of 1%, 5%, or 10% in either direction (greater than or less than) of the number unless otherwise stated or otherwise evident from the context (except where such number would be less than 0% or exceed 100% of a possible value).

As used herein, the term “adjuvant chemotherapy” refers to medicines administered after surgery for the treatment of cancer (e.g., debulking surgery also referred to as cytoreduction). Adjuvant chemotherapy is designed to prevent recurrence of the disease, particularly distant recurrence. “Neoadjuvant chemotherapy” refers to medicines that are administered before surgery for the treatment of cancer.

As used herein, the “administration” of an agent, drug, or peptide to a subject includes any route of introducing or delivering to a subject a compound to perform its intended function. Administration can be carried out by any suitable route, including orally, intranasally, parenterally (intravenously, intramuscularly, intraperitoneally, or subcutaneously), or topically. Administration includes self-administration and the administration by another.

As used herein, the term “biological sample” means sample material derived from living cells. Biological samples may include tissues, cells, protein or membrane extracts of cells, and biological fluids (e.g., ascites fluid or cerebrospinal fluid (CSF)) isolated from a subject, as well as tissues, cells and fluids present within a subject. Biological samples of the present technology include, but are not limited to, samples taken from breast tissue, renal tissue, the uterine cervix, the endometrium, the head or neck, the gallbladder, parotid tissue, the prostate, the brain, the pituitary gland, kidney tissue, muscle, the esophagus, the stomach, the small intestine, the colon, the liver, the spleen, the pancreas, thyroid tissue, heart tissue, lung tissue, the bladder, adipose tissue, lymph node tissue, the uterus, ovarian tissue, adrenal tissue, testis tissue, the tonsils, thymus, blood, hair, buccal, skin, serum, plasma, CSF, semen, prostate fluid, seminal fluid, urine, feces, sweat, saliva, sputum, mucus, bone marrow, lymph, and tears. Biological samples can also be obtained from biopsies of internal organs. Biological samples can be obtained from subjects for diagnosis or research or can be obtained from non-diseased individuals, as controls or for basic research. Samples may be obtained by standard methods including, e.g., venous puncture and surgical biopsy. In certain embodiments, the biological sample is an adipose tissue.

As used herein, a “control” or “reference sample” is an alternative sample used in an experiment for comparison purpose. A control can be “positive” or “negative.” For example, where the purpose of the experiment is to determine a correlation of the efficacy of a therapeutic agent for the treatment for a particular type of disease, a positive control (a compound or composition known to exhibit the desired therapeutic effect) and a negative control (a subject or a sample that does not receive the therapy or receives a placebo) are typically employed. In some embodiments, a reference sample is obtained from a healthy control subject, or contains a predetermined level of the proteins or markers that are being measured in the sample (e.g., cGAS⁺ micronuclei, STING protein expression, ENPP1 protein expression), or normal tissue corresponding to the tumor sample. As used herein, the terms “corresponding tissue,” “corresponding normal tissue,” “corresponding healthy tissue,” or “normal tissue corresponding to the tumor sample,” refer to tissue that is of the same origin of the tumor of interest. For example, if the tumor of interest is breast cancer, the corresponding normal tissue is normal or healthy breast tissue. The corresponding tissue may be from the same or a different individual.

As used herein, the term “effective amount” refers to a quantity sufficient to achieve a desired therapeutic and/or prophylactic effect, e.g., an amount which results in the prevention of, or a decrease in a disease or condition described herein or one or more signs or symptoms associated with a disease or condition described herein. In the context of therapeutic or prophylactic applications, the amount of a composition administered to the subject will vary depending on the composition, the degree, type, and severity of the disease and on the characteristics of the individual, such as general health, age, sex, body weight and tolerance to drugs. The skilled artisan will be able to determine appropriate dosages depending on these and other factors. The compositions can also be administered in combination with one or more additional therapeutic compounds. In the methods described herein, the therapeutic compositions may be administered to a subject having one or more signs or symptoms of a disease or condition described herein. As used herein, a “therapeutically effective amount” of a composition refers to composition levels in which the physiological effects of a disease or condition are ameliorated or eliminated. A therapeutically effective amount can be given in one or more administrations.

As used herein, “expression” includes one or more of the following: transcription of the gene into precursor mRNA; splicing and other processing of the precursor mRNA to produce mature mRNA; mRNA stability; translation of the mature mRNA into protein (including codon usage and tRNA availability); and glycosylation and/or other modifications of the translation product, if required for proper expression and function.

As used herein, the term “high-power field” or “HPF,” refers to the field of view under the maximum magnification power of the objective being used in a microscope.

As used herein, “metastasis” refers to the spread of cancer from its primary site to neighboring tissues or distal locations in the body. Cancer cells (including cancer stem cells) can break away from a primary tumor, penetrate lymphatic and blood vessels, circulate through the bloodstream, and grow in normal tissues elsewhere in the body. Metastasis is a sequential process, contingent on tumor cells (or cancer stem cells) breaking off from the primary tumor, traveling through the bloodstream or lymphatics, and stopping at a distant site. Once at another site, cancer cells re-penetrate through the blood vessels or lymphatic walls, continue to multiply, and eventually form a new tumor (metastatic tumor). In some embodiments, this new tumor is referred to as a metastatic (or secondary) tumor.

As used herein, the term “pharmaceutically-acceptable carrier” is intended to include any and all solvents, dispersion media, coatings, antibacterial and antifungal compounds, isotonic and absorption delaying compounds, and the like, compatible with pharmaceutical administration. Pharmaceutically-acceptable carriers and their formulations are known to one skilled in the art and are described, for example, in Remington's Pharmaceutical Sciences (20^{th} edition, ed. A. Gennaro, 2000, Lippincott, Williams & Wilkins, Philadelphia, PA.).

As used herein, “prevention” or “preventing” of a disorder or condition refers to a compound that, in a statistical sample, reduces the occurrence of the disorder or condition in the treated sample relative to an untreated control sample, or delays the onset or reduces the severity of one or more symptoms of the disorder or condition relative to the untreated control sample.

As used herein, the term “separate” therapeutic use refers to an administration of at least two active ingredients at the same time or at substantially the same time by different routes.

As used herein, the term “sequential” therapeutic use refers to administration of at least two active ingredients at different times, the administration route being identical or different. More particularly, sequential use refers to the whole administration of one of the active ingredients before administration of the other or others commences. It is thus possible to administer one of the active ingredients over several minutes, hours, or days before administering the other active ingredient or ingredients. There is no simultaneous treatment in this case.

As used herein, the term “simultaneous” therapeutic use refers to the administration of at least two active ingredients by the same route and at the same time or at substantially the same time.

As used herein, “solid tumor” refers to all neoplastic cell growth and proliferation, and all pre-cancerous and cancerous cells and tissues, except for hematologic cancers such as lymphomas, leukemias, and multiple myeloma. Examples of solid tumors include, but are not limited to: soft tissue sarcoma, such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor and other bone tumors (e.g., osteosarcoma, malignant fibrous histiocytoma), leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, brain/CNS tumors (e.g., astrocytoma, glioma, glioblastoma, childhood tumors, such as atypical teratoid/rhabdoid tumor, germ cell tumor, embryonal tumor, ependymoma) medulloblastoma, craniopharyngioma, ependymoma, pinealoma, hemangioblastoma, acoustic neuroma, oligodendroglioma, meningioma, melanoma, neuroblastoma, and retinoblastoma. Some of the most common solid tumors for which the compositions and methods of the present disclosure would be useful include: head-and-neck cancer, rectal adenocarcinoma, glioma, medulloblastoma, urothelial carcinoma, pancreatic adenocarcinoma, uterine (e.g., endometrial cancer, fallopian tube cancer) ovarian cancer, cervical cancer prostate adenocarcinoma, non-small cell lung cancer (squamous and adenocarcinoma), small cell lung cancer, melanoma, breast carcinoma, bladder cancer, ductal carcinoma in situ, renal cell carcinoma, and hepatocellular carcinoma, adrenal tumors (e.g., adrenocortical carcinoma), esophageal, eye (e.g., melanoma, retinoblastoma), gallbladder, gastrointestinal, Wilms' tumor, heart, head and neck, laryngeal and hypopharyngeal, oral (e.g., lip, mouth, salivary gland), nasopharyngeal, neuroblastoma, peritoneal, pituitary, Kaposi's sarcoma, small intestine, stomach, testicular, thymus, thyroid, parathyroid, vaginal tumor, and the metastases of any of the foregoing.

As used herein, the terms “subject,” “individual,” or “patient” can be an individual organism, a vertebrate, a mammal, or a human. In some embodiments, the subject, individual, or patient is a human.

As used herein, a “synergistic therapeutic effect” in some embodiments reflects a greater-than-additive therapeutic effect that is produced by a combination of at least two agents, and which exceeds that which would otherwise result from the individual administration of the agents. In some embodiments, a “synergistic therapeutic effect” reflects an enhanced therapeutic effect that is produced by a combination of at least two agents relative to the individual administration of the agents. For example, lower doses of one or more agents may be used in treating a disease or disorder, resulting in increased therapeutic efficacy and decreased side-effects.

“Treating,” “treat,” or “treatment” as used herein covers the treatment of a disease or disorder described herein, in a subject, such as a human, and includes: (i) inhibiting a disease or disorder, i.e., arresting its development; (ii) relieving a disease or disorder, i.e., causing regression of the disorder; (iii) slowing progression of the disorder; and/or (iv) inhibiting, relieving, or slowing progression of one or more symptoms of the disease or disorder. In some embodiments, treatment means that the symptoms associated with the disease are, e.g., alleviated, reduced, cured, or placed in a state of remission. In some embodiments, “inhibiting,” means reducing or slowing the growth of a tumor. In some embodiments, the inhibition of tumor growth may be, for example, by 5% or more, 10% or more, 20% or more, 30% or more, 40% or more, 50% or more, 60% or more, 70% or more, 80% or more, or 90% or more. In some embodiments, the inhibition may be complete. For example, a subject is successfully “treated” for cancer, if, after receiving a therapeutic amount of the STING inhibitor, STING agonist, cGAS inhibitor, ENPP1 inhibitor, ENPP1 inhibitor in combination with an NT5E inhibitor, and/or cancer therapy (e.g., chemotherapy, radiation therapy, immunotherapy), etc., of the present technology according to the methods described herein, the subject shows, for example, observable and/or measurable increased survival, decreased metastasis, reduced tumor burden, reduced tumor relapse during post-debulking adjuvant chemotherapy in the subject, reduced number of cancer cells, reduced tumor size, eradication of the tumor, inhibition of cancer cell infiltration into peripheral organs, inhibition or stabilization of tumor growth, and/or stabilization or improvement of the quality of life in the subject.

It is also to be appreciated that the various modes of treatment of disorders as described herein are intended to mean “substantial,” which includes total but also less than total treatment, and wherein some biologically or medically relevant result is achieved. The treatment may be a continuous prolonged treatment for a chronic disease or a single, or few time administrations for the treatment of an acute condition.

### II. INTRODUCTION

A. Pharmacologic STING Inhibition Suppresses Cancer Metastasis

Chromosomal instability (CIN) is a hallmark of human cancer and it is associated with widespread therapeutic resistance, immune evasion, and metastasis. Unlike many targetable genomic alterations in cancer, CIN remains a major therapeutic challenge given the lack of defined pathways that are shared among chromosomally unstable tumors.

Using human xenograft tumor models, it has recently been shown that CIN promotes tumor progression and metastasis through the chronic activation of cancer cell-intrinsic inflammatory signaling. Errors in chromosome segregation during mitosis—a defining hallmark of CIN—generate micronuclei. Micronuclear envelopes are rupture prone, leading to the exposure of their enclosed genomic double-stranded DNA (dsDNA) to the cytosol. Cytosolic dsDNA activates the cyclic GMP-AMP synthase (cGAS)-stimulator of interferon genes (STING) or “cGAS-STING” pathway, which has evolved to detect the early steps of viral infection shortly after viral DNA entry. Upon sensing cytosolic dsDNA, cGAS catalyzes the formation of the cyclic dinucleotide, cGAMP, which binds simulator of interferon genes (STING), leading to downstream inflammatory signaling. In parallel, STING activation by cGAMP promotes autophagy, which leads to STING degradation, thereby constituting a critical negative feedback mechanism.

In normal cells, cGAS-STING engagement induces cellular senescence and a pro-inflammatory immune response through the acute induction of interferon (IFN) signaling. This observation has provided the rationale for the development of STING agonists. This class of therapies is being tested in combination with immune checkpoint blockade in patients with advanced cancers with the goal of potentiating anti-tumor immune effects. Yet, by virtue of their persistent exposure to cytosolic dsDNA, chromosomally unstable cancer cells have evolved to suppress robust induction of IFN-stimulated genes (ISGs) downstream of STING along with its deleterious consequences, and instead activate STING-dependent noncanonical NF-κB signaling to promote migration, invasion, and metastasis. Whether this relationship can be therapeutically exploited remains to be shown. Importantly, given the dichotomous roles of STING as both a stimulator of the immune system and a promoter of tumor progression, it remains unclear whether its inhibition in chromosomally unstable tumors, where it is unremittingly activated, would represent a viable therapeutic strategy.

The technology of the present disclosure relates in part to the discovery that CIN enables tolerance to STING signaling due to ongoing exposure of cytosolic DNA in micronuclei, promoting constitutive cGAS activation. Mechanistically, cGAMP-mediated STING stimulation triggers its degradation, resulting in low steady-state levels that are sufficient to drive metastasis but insufficient to mount an interferon response. Pharmacologic inhibition of STING reduces migration and invasion, and dampens baseline inflammatory signaling in cancer cells. Strikingly, as demonstrated herein, STING inhibitors suppress metastasis in syngeneic models of melanoma, breast, and colorectal cancers. As demonstrated herein, in human tumors, cGAS localization to micronuclei is associated with evidence for high STING turnover, increased genomic copy number alterations, and poor prognosis. Thus, inhibition of chronic STING signaling represents a viable therapeutic strategy in chromosomally unstable metastatic cancers.

B. Metastasis and Immune Evasion from Extracellular cGAMP Hydrolysis

As mentioned above, CIN in cancer is associated with metastasis, immune evasion, and therapeutic resistance. Chromosome segregation errors lead to the formation of micronuclei and micronuclear envelopes are highly rupture-prone, often exposing genomic double-stranded DNA (dsDNA) to the cytosol. Cytosolic dsDNA is sensed by cGAS, which upon binding to its substrate, catalyzes the formation of the cyclic dinucleotide, cGAMP. cGAMP is a potent immune-stimulatory molecule that promotes inflammatory signaling in a manner dependent on its downstream effector STING. Chromosomally unstable cancer cells have evolved to cope with chronic cGAS-STING activation by silencing downstream type I interferon signaling whilst co-opting NF-κB-dependent transcription to spread to distant organs. However, cGAMP is also readily exported to the extracellular space where it can promote anti-tumor immune responses by activating STING in neighboring host cells. How cancer cells co-opt inflammatory signaling while simultaneously evading immune surveillance remains unknown. Furthermore, the role of extracellular cGAMP in metastasis and immune evasion in the context of CIN remains poorly understood. Because cancer cells rely in large part on CIN and its downstream inflammatory signaling to spread to distant organs, identifying mechanisms by which tumor cells cope with inflammation represents an attractive therapeutic opportunity.

The disclosure of the present technology relates in part to the discovery that the ectonucleotidase, ectonucleotide pyrophosphatase/phosphodiesterase 1 (ENPP1), promotes metastasis by selectively degrading extracellular cGAMP, an immune stimulatory metabolite whose breakdown products include the immune suppressor, adenosine. As demonstrated herein, ENPP1 depletion restores tumor immune infiltration, suppresses metastasis, and potentiates tumor response to immune checkpoint blockade (ICB) therapy. Conversely, its overexpression renders otherwise sensitive tumors completely resistant to ICB. In human cancers, ENPP1 expression correlates with reduced immune cell infiltration, increased metastasis, and resistance to anti-PD1/PD-L1 therapy. Thus, cGAMP hydrolysis by ENPP1 enables metastatic cancer cells to transmute an immune stimulatory pathway into an immune suppressive mechanism supporting tumor progression.

### III. cGAS⁺ MICRONUCLEI AS A BIOMARKER FOR CHROMOSOMAL INSTABILITY (CIN)

The ability to accurately detect and quantitatively assess chromosomal instability (CIN) in cancer is of tremendous interest within research and clinical settings as it can aid in understanding the role of CIN in cancer pathogenesis, its implications for patient prognosis, and for developing novel therapeutics capable of exploiting CIN. However, a reliable test for the identification of CIN in cancer has yet to be developed. Most importantly, CIN, as defined by the ongoing rate or frequency of chromosome missegregation events cannot be accurately inferred by sequencing approaches which are best suited to define genomic copy number heterogeneity and as such report the state of chromosomal alterations. And while CIN leads to aneuploidy and genomic copy number heterogeneity, the two metrics do not always correlate and it is important to be able to identify methods that can infer (and measure) CIN in tumor samples for prognostication and predictive purposes.

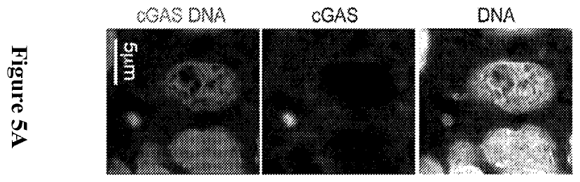

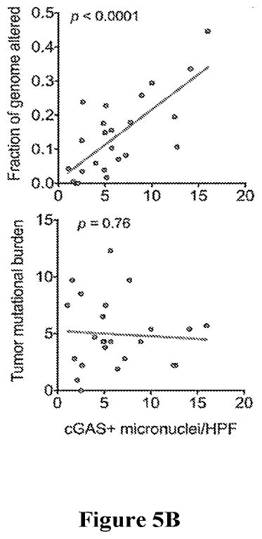

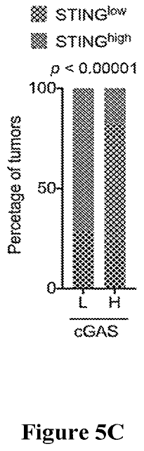

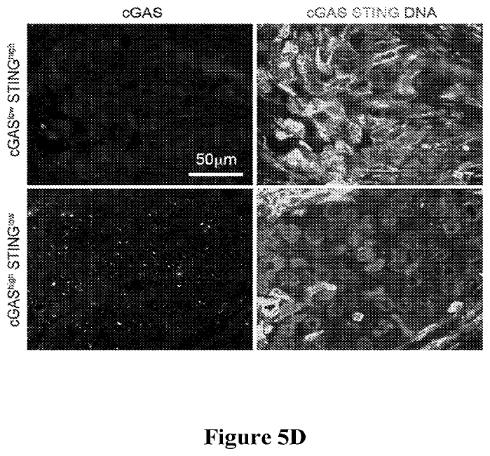

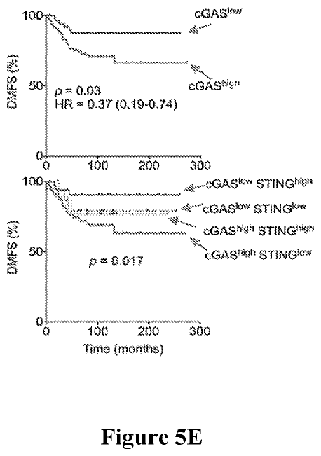

To address this need, as demonstrated herein, the inventors of the present technology found that in human cancer samples, cGAS is primarily localized to micronuclei and the frequency of micronuclear cGAS staining was highly correlated with genomic copy number alterations (R²=0.54, p<0.0001)—but not with the tumor mutational burden (R²=0.004, p=0.76) (FIG. 5B), suggesting that this staining pattern may serve as a surrogate marker for CIN in human cancer.

Accordingly, in some embodiments, the disclosure of the present technology relates to a method for detecting chromosomal instability in a tumor in a subject, comprising: obtaining one or more tissue sections of a tumor sample from a subject; and measuring the degree of chromosomal instability present in the tumor sample by contacting the tumor sample with an anti-cGAS antibody and measuring the present of cGAS⁺ micronuclei in the tumor sample.

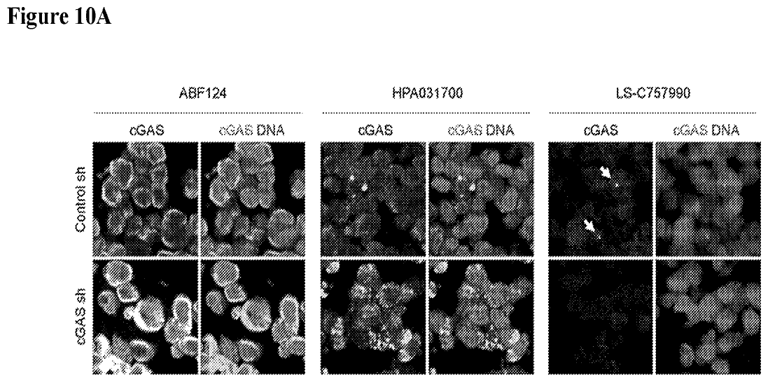

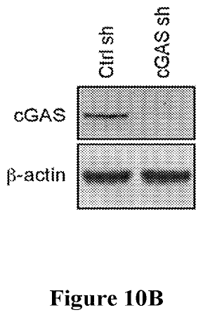

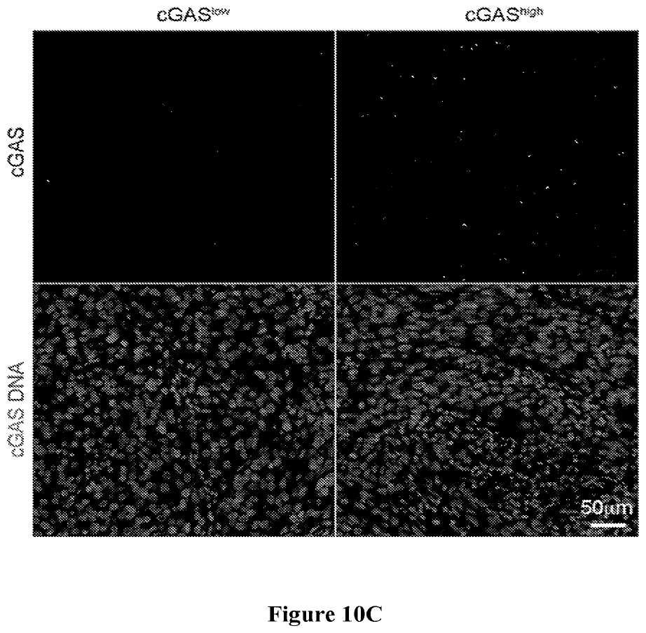

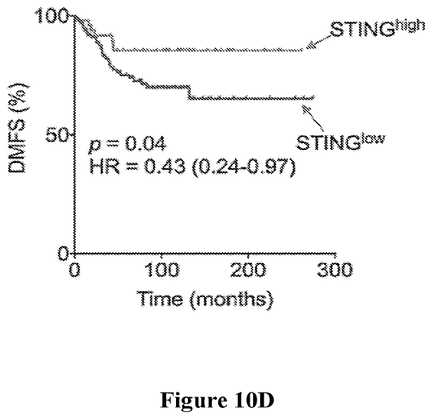

Quantifying the degree of chromosomal instability in an anti-cGAS antibody-stained tumor sample can be accomplished by methods known in the art. For example, in some embodiments, the number of cGAS⁺ micronuclei in a defined high-power field (HPF) can be quantified, such that, for example, a total of 5 or more cGAS⁺ micronuclei within the defined HPF (e.g., standard 20× high power field) indicates a high degree of chromosomal instability in a tumor sample. In some embodiments, semi-quantitative assessments may also be performed. In some embodiments, the degree of chromosomal instability may also be measured as a percentile in a large tumor cohort wherein a given tumor above the 33^{rd} percentile, for example, is considered to have a high degree of chromosomal instability, as is shown in FIG. 10C and FIGS. 5D-5E. In some embodiments, the degree of chromosomal instability in a tumor sample is measured as a fraction of cGAS⁺ micronuclei/primary nuclei, wherein a fraction above 8-10% is considered to indicate a high degree of chromosomal instability.

### IV. PERSONALIZED THERAPEUTIC APPROACHES FOR TARGETING THE CGAS-STING PATHWAY IN CANCER

As demonstrated by the experimental examples, the disclosure of the present technology identifies a genomic basis for tumor cell-intrinsic chronic inflammatory signaling arising from CIN and demonstrates that its pharmacologic inhibition is useful in methods for treating cancer.

Agents such as the cyclic dinucleotide analogs that activate the cGAS-STING pathway have been the subject of intense investigation due to their ability to promote IFN-dependent anti-tumor immune responses. As demonstrated herein, CIN enables tolerance to cGAS-STING signaling due to ongoing exposure of cytosolic dsDNA in micronuclei, promoting constitutive cGAS activation without a corresponding downstream IFN response. As such, chromosomally unstable tumors exhibit pre-existing resistance to STING agonists. Without wishing to be bound by theory, this may potentially explain the absence of their apparent efficacy in early stage clinical trials, particularly given the widespread CIN phenotype in metastatic cancers. In these tumors, STING triggers its own autophagy-mediated degradation, remaining at low steady-state levels that are sufficient to drive metastatic progression yet insufficient to induce IFN signaling. Interestingly, the autophagy-related function of STING predates its role as a stimulator of type I IFN. The data provided herein suggest that alleviating chronic STING stimulation and its subsequent degradation through cGAS inhibition restores IFN responsiveness downstream of STING and represents an alternative method to sensitize chromosomally unstable tumors to STING agonists.

The tunable nature of STING signaling has been demonstrated in multiple contexts and the outcome of activating this pathway is largely dependent on the strength and duration of the signaling output in response to cytosolic dsDNA. Acute and robust activation of STING leads to apoptosis, senescence, and anti-tumor immune responses. Chronic activation of this pathway, however, has been shown to promote tumorigenesis, therapeutic resistance, immune suppression, and metastasis. Therefore, personalizing the therapeutic approach of targeting the cGAS-STING pathway is central to the successful exploitation of this pathway in the clinic.

As shown in the experimental examples, the inverse correlation between the frequency of micronuclei with cGAS staining and cancer cell-intrinsic STING expression mirrors findings in cancer cell lines and, without wishing to be bound by theory, suggests that the apparent loss of STING protein in human cancers is most likely a manifestation of its chronic activation. In line with these findings, patients whose tumors displayed elevated cGAS staining and reduced cancer cell-intrinsic STING expression (cGAS^{high}STING^{low} tumors) had the worst prognosis and their tumors were amongst the most chromosomally unstable. Therefore, a mechanistic understanding underlying the use of cGAS-STING-based biomarkers enables a personalized therapeutic paradigm. For instance, patients whose tumors exhibit evidence for chronic cGAS-STING activation might benefit from STING inhibition. Conversely, tumors with low CIN, a paucity of micronuclei with cGAS staining, and increased tumor cell-STING expression might be sensitive to STING agonists as they are likely capable of mounting an effective cancer cell-intrinsic IFN response. Such a nuanced approach will be required for the successful delivery of cGAS-STING-directed therapies into the clinic. It is also relevant to a large cohort of patients with chromosomally unstable tumors and for whom there are few effective therapeutic options. The identification of chronic cGAS-STING signaling as a shared feature of tumors with CIN offers a heretofore-untapped opportunity for therapeutic intervention and recognizes chronic inflammation as an important target in cancer treatment.

Accordingly, in some embodiments, the disclosure of the present technology relates to methods for selecting subjects for treatment with a STING inhibitor based on the presence of a cGAS^{high}STING^{low} tumor and treating cancer in subjects having cGAS^{high}STING_{low} tumors with a therapeutically effective amount of a STING inhibitor. In some embodiments, the present technology provides a method for treating cancer associated with increased cGAS⁺ micronuclei and decreased STING protein expression (cGAS^{high}STING^{low}), in a subject in need thereof, comprising: detecting an increase in cGAS⁺ micronuclei and a decrease in cancer cell-specific STING protein expression in a tumor sample (cGAS_{high}STING^{low} tumor) obtained from a subject as compared to that observed in a reference sample, thereby detecting a cGAS^{high}STING^{low} tumor; and administering a therapeutically effective amount of a STING inhibitor to the subject for whom a cGAS^{high}STING^{low} tumor has been detected. In some embodiments, the present technology provides a method for selecting a subject for the treatment of cancer with a STING inhibitor, comprising: detecting an increase in cGAS⁺ micronuclei and a decrease in STING protein expression in a tumor sample (cGAS^{high}STING^{low} tumor) obtained from a subject as compared to that observed in a reference sample; and selecting the subject for whom a cGAS^{high}STING^{low} tumor sample has been detected as a subject for the treatment of cancer with a STING inhibitor. Reference samples may be obtained from a healthy control subject, or normal tissue corresponding to the tumor sample, or may contain a predetermined level of cGAS⁺ micronuclei and STING protein expression.

In some embodiments, detecting cGAS⁺ micronuclei comprises contacting the tumor sample with an anti-cGAS antibody and measuring the presence of cGAS⁺ micronuclei in the tumor sample, and detecting cancer cell-specific STING protein expression comprises contacting the tumor sample with an anti-STING antibody and measuring STING expression levels in the tumor sample.

Any STING inhibitor may be used in the methods of the present technology. In some embodiments, the STING inhibitor is selected from the group consisting of C-176, C-178, compound H-151, tetrahydroisoquinolone acetic acids, 9-nitrooleate, 10-nitrooleate, nitro conjugated linoleic acid), nitrofurans, Astin C, analogue M11, and any combination thereof.

In some embodiments, the method further comprise separately, sequentially, or simultaneously administering to the subject any one or more immune checkpoint blocking agents. In some embodiments, the immune checkpoint blocking agent is selected from the group consisting of anti-PD-L1 antibody, anti-PD-1 antibody, anti-CTLA-4 antibody, ipilimumab, nivolumab, pidilizumab, lambrolizumab, pembrolizumab, atezolizumab, avelumab, durvalumab, MPDL3280A, BMS-936559, MEDI-4736, MSB 00107180, LAG-3, TIM3, B7-H3, B7-H4, TIGIT, AMP-224, MDX-1105, arelumab, tremelimumab, IMP321, MGA271, BMS-986016, lirilumab, urelumab, PF-05082566, IPH2101, MEDI-6469, CP-870,893, Mogamulizumab, Varlilumab, Galiximab, AMP-514, AUNP 12, Indoximod, NLG-919, INCB024360, CD80, CD86, ICOS, DLBCL inhibitors, BTLA, PDR001, and any combination thereof.

The methods of the present technology may be used to treat any solid malignant tumor, including but not limited to melanoma, breast cancer, colorectal cancer, lung cancer, prostate cancer, bladder cancer, pancreatic cancer, ovarian cancer, squamous cell carcinoma of the skin, Merkel cell carcinoma, gastric cancer, liver cancer, thyroid cancer, and sarcoma.

Benefits of the present methods of treatment include but are not limited to one or more of the following: increased survival, decreased metastasis, reduced tumor burden, reduced tumor relapse during post-debulking adjuvant chemotherapy, reduction of the number of cancer cells, reduction of the tumor size, eradication of tumor, inhibition of cancer cell infiltration into peripheral organs, inhibition or stabilization of tumor growth, and stabilization or improvement of quality of life in the subject.

In addition, the disclosure of the present technology provides a method for re-sensitizing tumors to treatment with STING agonists by administering a cGAS inhibitor prior to administering a STING agonist to subjects identified as having cGAS^{high}STING^{low} tumors. This is of critical importance to therapies that aim to activate innate immune signaling (in particular interferon response) by activating STING in the tumor. This class includes STING agonists (activators), which have thus far exhibited limited clinical efficacy in early stage clinical trials. Without wishing to be bound by theory, it is thought that this is due to pre-existing and adaptive resistance induced by CIN (i.e., cGAS^{high}STING^{low}) tumors which is associated with suppressed type I interferon signaling as well as suppressed anti-tumor immune response. In addition to being able to predict patients that would be resistant to STING agonists (i.e., cGAS^{high}STING^{low}), it is proposed that reducing chronic cGAS activation through cGAS inhibition can restore sensitivity to STING activating agents and therapies by restoring type I interferon responsiveness. Accordingly, in some embodiments, the present technology provides a method for increasing tumor sensitivity to treatment with a STING agonist in a subject in need thereof, comprising: detecting an increase in cGAS⁺ micronuclei and a decrease in STING protein expression in a tumor sample (cGAS^{high}STING^{low} tumor) obtained from a subject as compared to that observed in a reference sample; and administering a therapeutically effective amount of a cGAS inhibitor to the subject for whom a cGAS^{high}STING^{low} tumor has been detected prior to administering a therapeutically effective amount of a STING agonist to the subject.

Any known cGAS inhibitor may be used in the methods of the present technology. In some embodiments, the cGAS inhibitor is selected from the group consisting of J014 or analogs thereof, G150 or analogs thereof, RU.521, suramin, PF-06928215, hydroxychloroquine, quinacrin, and any combination thereof.

Any known STING agonist may be used in the methods of the present technology. In some embodiments, the STING agonist is selected from the group consisting of c-di-AMP, c-di-GMP, diABZIs, 3′3′-cGAMP, 2′3′-cGAMP, 5,6-dimethylxanthenone-4-acetic acid (DMXAA), macrocycle-bridged STING agonist E7766, GSK3745417, MK-1454, MK-2118, ADU-S100, SB11285, BMS-98630, and any combination thereof.

In some embodiments, the STING agonist is administered within about 24 hours, about 48 hours, about 72 hours, about one week, or about two weeks or more of the cGAS inhibitor. In some embodiments, the STING agonist is administered concurrently with the cGAS inhibitor (e.g., at the same time or within less than one hour of each other).

In some embodiments, the methods of treating cancer may further comprise separately, sequentially, or simultaneously administering an additional therapy to the subject, including but not limited to, immune checkpoint blockade agents, radiation therapy, or chemotherapy.

In addition, as discussed above, in some embodiments, cGAS⁺ micronuclei and cancer-specific STING protein expression levels may be used as prognostic indicators for cancer patients. For example, as a prognostic indicator, cGAS^{high}STING^{low} tumors are associated with poor prognosis and increased metastasis, whereas cGAS^{low}STING^{high} tumors are associated with a favorable prognosis.

### V. PERSONALIZED THERAPEUTIC APPROACHES FOR TARGETING ENPP1 IN CANCER

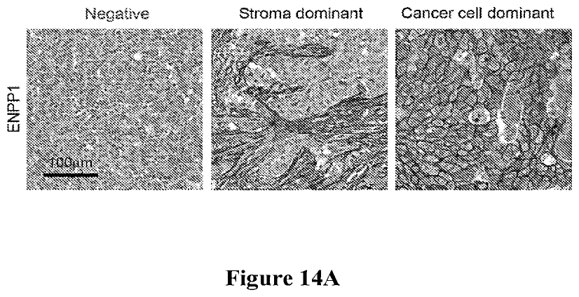

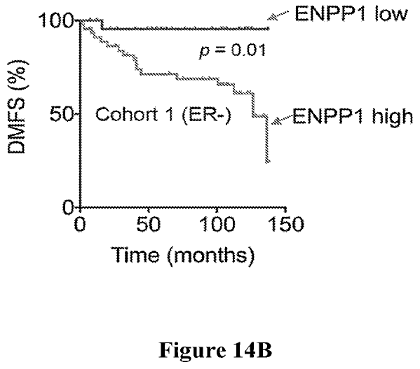

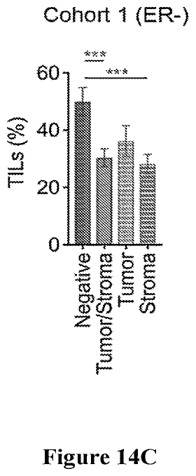

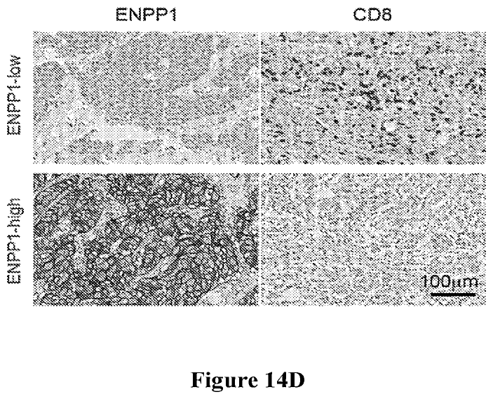

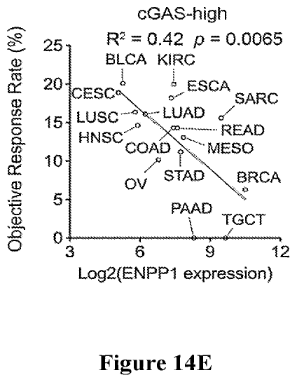

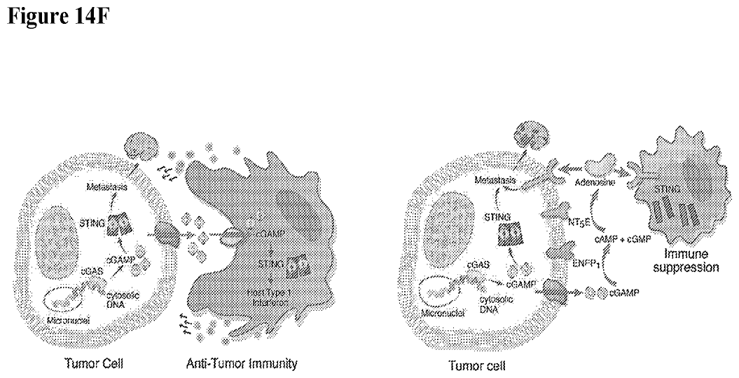

The experimental results presented herein uncover an adaptive mechanism by which chromosomally unstable tumors co-opt cancer cell-intrinsic cGAS-STING signaling, arising from chronic exposure to cytosolic dsDNA, without eliciting anti-tumor immune surveillance (FIG. 14F). By virtue of their constant exposure to cytosolic dsDNA, chromosomally unstable cancer cells must address the consequences of cGAMP leakage into the extracellular space and its potential uptake by cells in the tumor microenvironment. Through their ability to degrade cGAMP selectively in the extracellular environment, tumor cells can maintain relatively high levels of this metabolite in the intracellular compartment while minimizing paracrine STING activation in neighboring immune cells (FIG. 14F).

Furthermore, extracellular cGAMP hydrolysis by ENPP1 generates the substrate for adenosine production converting an immune stimulatory pathway into an immune suppressive mechanism that promotes tumor progression (FIG. 14F). Inhibition of extracellular adenosine production and signaling is currently being investigated at the pre-clinical and clinical stages. ENPP1 inhibition would achieve the dual purpose of reducing extracellular adenosine while simultaneously increasing the extracellular levels of the immunotransmitter cGAMP. These findings highlight an important STING-independent function for tumor cGAS and suggests that in the presence of ENPP1, high tumor cGAS activity might in fact be paradoxically immune suppressive.

STING agonists have been the focus of intense investigation given their ability to elicit anti-tumor immunity through type I interferon signaling. Inhibition of ENPP1 is distinct from direct pharmacologic activation of STING in a number of important ways. First, ENPP1 tilts the relative balance of STING activation away from cancer cells, where it promotes metastatic progression, and towards host cells where it potentiates anti-tumor immunity. STING agonists indiscriminately activate STING in both cancer cells and the host promoting dichotomous outcomes. Second, inhibition of cGAMP hydrolysis by ENPP1 would primarily impact cGAMP concentrations at the microscopic scales relevant to paracrine tumor cell-host cell interactions. This, along with the short half-life of cGAMP in circulation, is likely to minimize systemic side effects of ENPP1 inhibition, offering a larger therapeutic window. Third, ENPP1 is selectively upregulated in metastatic and chromosomally unstable tumor cells and thus a systemic ENPP1 inhibitor would interfere with the ability of disseminated tumor cells to evade immune surveillance arising from CIN. The data presented herein highlights the therapeutic utility of selectively targeting cancer cell dependencies on CIN and the mechanism by which they have evolved to tolerate it.

Accordingly, in some embodiments, the disclosure of the present technology relates to methods for selecting subjects for treatment with an ENPP1 inhibitor based on the presence of either increased ENPP1 protein expression levels in a tumor sample or increased cGAS^{T} micronuclei and increased ENPP1 expression levels in a tumor (cGAS^{high}ENPPh1^{high} tumor), as compared to that observed in a reference sample, and treating cancer in subjects having cGAS^{high}ENPP1^{high} tumors with a therapeutically effective amount of an ENPP1 inhibitor.

In some embodiments, the present technology provides a method for treating cancer associated with increased ENPP1 expression and/or increased cGAS⁺ micronuclei and increased ENPP1 (cGAS^{high}ENPP1^{high}) in a subject in need thereof, comprising: detecting the presence or absence of an increased ENPP1 protein expression level and/or cGAS⁺ micronuclei in a tumor sample obtained from a subject as compared to that observed in a reference sample; and administering a therapeutically effective amount of an ENPP1 inhibitor to the subject for whom an increased ENPP1 protein expression level or cGAS^{high}ENPP1^{high} tumor has been detected. In some embodiments, the present technology provides a method for selecting a subject for the treatment of cancer with an ENPP1 inhibitor, comprising: detecting the presence or absence of an increased ENPP1 protein expression level and/or a cGTAS^{high} ENPP1^{high} tumor in a tumor sample obtained from the subject; as compared to that observed in a reference sample and selecting the subject for whom an increased ENPP1 protein expression level and/or cGAS^{high}ENPP1^{high} tumor has been detected as a subject for the treatment of cancer with an ENPP1 inhibitor. Reference samples may be obtained from a healthy control subject, or normal tissue corresponding to the tumor sample, or may contain a predetermined level of ENPP1 protein expression and/or cGAS⁺ micronuclei.

In some embodiments, detecting ENPP1 protein expression comprises contacting the tumor sample with an anti-ENPP11 antibody and measuring ENPP1 expression levels in the tumor sample. In some embodiments, detecting cGAS⁺ micronuclei comprises contacting the tumor sample with an anti-cGAS antibody and measuring the presence of cGAS⁺ micronuclei in the tumor sample.

Any ENPP1 inhibitor may be used in the methods of the present technology. In some embodiments, the ENPP1 inhibitor is selected from the group consisting of α,β-metADP, α,β-metATP, 2-MeSADP, 2-MeSATP, bzATP, γ-S-α,β-metATP derivatives, ARL 67156, α-borano-β, γ-metATP derivatives, diadenosine boranophosphate derivatives, polyoxometalates [TiW11CoO40]⁸⁻, reactive blue 2 (RB2), quinazoline derivative, suramin, heparin, PPADS, biscoumarin derivative, oxadiazole derivatives, quinazoline derivative, triazole derivative, thioacetamide derivative, isoquinoline derivative, thiadiazolopyrimidinone derivative, STF-1084, thiazolobenzimidazolone derivative, sulfamate derivatives, SR 8314, MV626, MAVU-104, and any combination thereof.

In some embodiments, the method further comprises separately, sequentially, or simultaneously administering a therapeutically effective amount of an NT5E (CD73) inhibitor. In some embodiments, the subject may or may not have a tumor characterized by increased ENPP1 expression and/or increased cGAS' micronuclei and increased ENPP1 (cGAS^{high} ENPP1^{high}) as compared to that observed in a reference sample.

Any NT5E inhibitor may be used in the methods of the present technology. In some embodiments, the NT5E inhibitor is selected from the group consisting of α,β-methylene-ADP, PSB-12379, PSB-12489, AD680, 4-({5-[4-fluoro-1-(2H-indazol-6-yl)-1H-1,2,3-benzotriazol-6-yl]-1H-pyrazol-1-yl}methyl)benzonitrile, 4-({5-[4-chloro-1-(2H-indazol-6-yl)-1H-1,2,3-benzotriazol-6-yl]-1H-pyrazol-1-yl}methyl)benzonitrile, E5NT-02, E5NT-03, 5-fluorouridine-5′-O-[(phosphonomethyl)phosphonic acid], 4-benzoylcytidine-5′-O-[(phosphonomethyl)phosphonic acid], N⁴-[O-(4-benzyloxy)]-3-methyl-cytidine-5′-O-[(phosphonomethyl)phosphonic acid], N⁴-[O-(naphthalen-2-yl-methoxy)]-cytidine-5′-O-[(phosphonomethyl)phosphonic acid], substituted 5′-aminoadenosine derivatives, APCP, 2-trifluoromethyl-4,6-diarylquinolines, benzothiazine compounds, RR2-4, RR6, RR8-9, RR11, RR16, RR18, RR20-21, pyrazolo[3,4-b]pyridines, pyrrolo[2,3-b]pyridines, pyrido[2,3-d]pyrimidines, benzofuro[3,2-b]pyridines, (E)-N′-(1-(3-(4-fluorophenyl)-5-phenyl-4,5-dihydro-1H-pyrazol-1-yl)ethylidene)isonicotinohydrazide, and any combination thereof.

In some embodiments, the administration of a combination of an ENPP1 inhibitor and an NT5E inhibitor to a subject results in one or more of increased immune infiltration, suppression of metastasis, reduced tumor volume, treatment of primary tumors, increased immune activation against tumors, sensitization to immunotherapy, sensitization to radiation therapy, sensitization to chemotherapy, and sensitization to therapies that induce DNA damage or genomic instability as compared to the inhibition of either ENPP1 or NT5E alone. In some embodiments, the combination of an ENPP1 inhibitor and an NT5E inhibitor has a synergistic effect in this regard.

In some embodiments, the methods of treating cancer comprise separately, sequentially, or simultaneously administering a STING agonist and an NT5E inhibitor. Without wishing to be bound by theory, it is thought that because ENPP1 activates STING, the combination of a STING agonist and an NT5E inhibitor will have effects similar to those resulting from the administration of an ENPP1 inhibitor and an NT5E inhibitor in the treatment of cancer. In some embodiments, the subject may or may not have a tumor characterized by elevated ENPP1 levels as compared to a reference sample. In some embodiments, the administration of a combination of a STING agonist and an NT5E inhibitor to a subject results in one or more of increased immune infiltration, suppression of metastasis, reduced tumor volume, treatment of primary tumors, increased immune activation against tumors, sensitization to immunotherapy, sensitization to radiation therapy, sensitization to chemotherapy, and sensitization to therapies that induce DNA damage or genomic instability as compared to either the activation of STING or the inhibition of NT5E alone. In some embodiments, the combination of a STING agonist and an NT5E inhibitor has a synergistic effect in this regard.

In some embodiments, the methods of treating cancer may further comprise separately, sequentially, or simultaneously administering an additional therapy to the subject, including but not limited to, immune checkpoint blockade agents, radiation therapy, or chemotherapy.

The methods of the present technology may be used to treat any solid malignant tumor, including but not limited to melanoma, breast cancer, colorectal cancer, lung cancer, prostate cancer, bladder cancer, pancreatic cancer, ovarian cancer, squamous cell carcinoma of the skin, Merkel cell carcinoma, gastric cancer, liver cancer, thyroid cancer, and sarcoma.

Benefits of the present methods of treatment include but are not limited to one or more of the following: increased survival, decreased metastasis, reduced tumor burden, reduced tumor relapse during post-debulking adjuvant chemotherapy, reduction of the number of cancer cells, reduction of the tumor size, eradication of tumor, inhibition of cancer cell infiltration into peripheral organs, inhibition or stabilization of tumor growth, and stabilization or improvement of quality of life in the subject.

In addition, the disclosure of the present technology provides a method for sensitizing an otherwise resistant or unresponsive tumor characterized by a low ENPP1 to cGAS expression ratio to immune checkpoint blockade therapy. Accordingly, in some embodiments, the present technology provides a method for treating cancer with an immune checkpoint blockade agent in a subject in need thereof, comprising: detecting the presence or absence of a low ENPP1 to cGAS expression ratio in a tumor sample obtained from a subject as compared to that observed in a reference sample; and administering a therapeutically effective amount of one or more immune checkpoint blockade agents to the subject for whom a low ENPP1 to cGAS expression ratio has been detected. The reference sample may be obtained from a healthy control subject, normal tissue corresponding to the tumor sample, or a sample containing a predetermined level of ENPP1 protein expression and cGAS⁺ micronuclei.

In some embodiments, detecting ENPP1 protein expression comprises contacting the tumor sample with an anti-ENPP1 antibody and measuring ENPP1 expression levels in the tumor sample. In some embodiments, detecting cGAS⁺ micronuclei comprises contacting the tumor sample with an anti-cGAS antibody and measuring the presence of cGAS⁺ micronuclei in the tumor sample.

In some embodiments, the immune checkpoint blocking agent is selected from the group consisting of anti-PD-L1 antibody, anti-PD-1 antibody, anti-CTLA-4 antibody, ipilimumab, nivolumab, pidilizumab, lambrolizumab, pembrolizumab, atezolizumab, avelumab, durvalumab, MPDL3280A, BMS-936559, MEDI-4736, MSB 00107180, LAG-3, TIM3, B7-H3, B7-H4, TIGIT, AMP-224, MDX-1105, arelumab, tremelimumab, IMP321, MGA271, BMS-986016, lirilumab, urelumab, PF-05082566, IPH2101, MEDI-6469, CP-870,893, Mogamulizumab, Varlilumab, Galiximab, AMP-514, AUNP 12, Indoximod, NLG-919, INCB024360, CD80, CD86, ICOS, DLBCL inhibitors, BTLA, PDR001, and any combination thereof.

In some embodiments, the methods of treating cancer may further comprise separately, sequentially, or simultaneously administering an additional therapy to the subject, including but not limited to, radiation therapy, and/or chemotherapy.

The methods of the present technology may be used to treat any solid malignant tumor, including but not limited to melanoma, breast cancer, colorectal cancer, lung cancer, prostate cancer, bladder cancer, pancreatic cancer, ovarian cancer, squamous cell carcinoma of the skin, Merkel cell carcinoma, gastric cancer, liver cancer, thyroid cancer, and sarcoma.

Benefits of the present methods of treatment include but are not limited to one or more of the following: increased survival, decreased metastasis, reduced tumor burden, reduced tumor relapse during post-debulking adjuvant chemotherapy, reduction of the number of cancer cells, reduction of the tumor size, eradication of tumor, inhibition of cancer cell infiltration into peripheral organs, inhibition or stabilization of tumor growth, and stabilization or improvement of quality of life in the subject.

### VI. MODES OF ADMINISTRATION AND EFFECTIVE DOSAGES

Any method known to those in the art for contacting a cell, organ, or tissue with a STING inhibitor, STING agonist, cGAS inhibitor, or ENPP1 inhibitor composition may be employed. Suitable methods include in vitro, ex vivo, or in vivo methods. In vivo methods typically include the administration of a STING inhibitor, STING agonist, cGAS inhibitor, or ENPP1 inhibitor, such as those described above, to a mammal, suitably a human. When used in vivo for therapy, the STING inhibitor, STING agonist, cGAS inhibitor, or ENPP1 inhibitor is administered to the subject in effective amounts (i.e., amounts that have desired therapeutic effect). The dose and dosage regimen will depend upon the degree of the disease symptoms in the subject, the characteristics of the particular STING inhibitor, STING agonist, cGAS inhibitor, or ENPP1 inhibitor, e.g., its therapeutic index, the subject, and the subject's history.

The effective amount may be determined during pre-clinical trials and clinical trials by methods familiar to physicians and clinicians. An effective amount of a STING inhibitor, STING agonist, cGAS inhibitor, or ENPP1 inhibitor composition useful in the methods may be administered to a mammal in need thereof by any of a number of well-known methods for administering pharmaceutical compounds. The STING inhibitor, STING agonist, cGAS inhibitor, or ENPP1 inhibitor composition may be administered systemically or locally.

The STING inhibitor, STING agonist, cGAS inhibitor, or ENPP1 inhibitor can be incorporated into pharmaceutical compositions for administration, singly or in combination, to a subject for the treatment or prevention of a disorder described herein. Such compositions typically include the active agent and a pharmaceutically acceptable carrier. As used herein the term “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions.

Pharmaceutical compositions are typically formulated to be compatible with its intended route of administration. Examples of routes of administration include parenteral (e.g., intravenous, intradermal, intraperitoneal or subcutaneous), oral, inhalation, transdermal (topical), intraocular, iontophoretic, and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose via 1s made of glass or plastic. For convenience of the patient or treating physician, the dosing formulation can be provided in a kit containing all necessary equipment (e.g., via is of drug, vials of diluent, syringes and needles) for a treatment course (e.g., 7 days of treatment).

In some embodiments, the STING inhibitor, STING agonist, cGAS inhibitor, or ENPP1 inhibitor described herein is administered by a parenteral route. In some embodiments, the STING inhibitor, STING agonist, cGAS inhibitor, or ENPP1 inhibitor is administered by a topical route.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, a composition for parenteral administration must be sterile and should be fluid to the extent that easy syringability exists. It should be stable under the conditions of manufacture and storage and must be preserved against the contaminating action of microorganisms such as bacteria and fungi.

The STING inhibitor, STING agonist, cGAS inhibitor, or ENPP1 inhibitor compositions described herein can include a carrier, which can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thiomerasol, and the like. Glutathione and other antioxidants can be included to prevent oxidation. In many cases, isotonic agents are included, for example, sugars, polyalcohols such as mannitol, sorbitol, or sodium chloride in the composition. Prolonged absorption of the injectable compositions can be brought about by including in the composition an agent which delays absorption, for example, aluminum monostearate or gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, typical methods of preparation include vacuum drying and freeze drying, which can yield a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the STING inhibitor, STING agonist, cGAS inhibitor, or ENPP1 inhibitor compositions of the present technology can be delivered in the form of an aerosol spray from a pressurized container or dispenser which contains a suitable propellant, e.g., a gas such as carbon dioxide, or a nebulizer. Such methods include those described in U.S. Pat. No. 6,468,798.

Systemic administration of a STING inhibitor, STING agonist, cGAS inhibitor, or ENPP1 inhibitor composition of the present technology as described herein can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays. For transdermal administration, the active compounds are formulated into ointments, salves, gels, or creams as generally known in the art. In one embodiment, transdermal administration may be performed by iontophoresis.

A STING inhibitor, STING agonist, cGAS inhibitor, or ENPP1 inhibitor composition of the present technology can be formulated in a carrier system. The carrier can be a colloidal system. The colloidal system can be a liposome, a phospholipid bilayer vehicle. In one embodiment, the therapeutic STING inhibitor, STING agonist, cGAS inhibitor, or ENPP1 inhibitor composition is encapsulated in a liposome while maintaining structural integrity. As one skilled in the art would appreciate, there are a variety of methods to prepare liposomes. (See Lichtenberg et al., Methods Biochem. Anal., 33:337-462 (1988); Anselem et al., Liposome Technology, CRC Press (1993)). Liposomal formulations can delay clearance and increase cellular uptake (See Reddy, Ann. Pharmacother., 34(7-8):915-923 (2000)). An active agent can also be loaded into a particle prepared from pharmaceutically acceptable ingredients including, but not limited to, soluble, insoluble, permeable, impermeable, biodegradable or gastroretentive polymers or liposomes. Such particles include, but are not limited to, nanoparticles, biodegradable nanoparticles, microparticles, biodegradable microparticles, nanospheres, biodegradable nanospheres, microspheres, biodegradable microspheres, capsules, emulsions, liposomes, micelles and viral vector systems.

The carrier can also be a polymer, e.g., a biodegradable, biocompatible polymer matrix. In one embodiment, the STING inhibitor, STING agonist, cGAS inhibitor, or ENPP1 inhibitor can be embedded in the polymer matrix, while maintaining protein integrity. The polymer may be natural, such as polypeptides, proteins or polysaccharides, or synthetic, such as poly α-hydroxy acids. Examples include carriers made of, e.g., collagen, fibronectin, elastin, cellulose acetate, cellulose nitrate, polysaccharide, fibrin, gelatin, and combinations thereof. In one embodiment, the polymer is poly-lactic acid (PLA) or copoly lactic/glycolic acid (PGLA). The polymeric matrices can be prepared and isolated in a variety of forms and sizes, including microspheres and nanospheres. Polymer formulations can lead to prolonged duration of therapeutic effect. (See Reddy, Ann. Pharmacother., 34(7-8):915-923 (2000)). A polymer formulation for human growth hormone (hGH) has been used in clinical trials. (See Kozarich and Rich, Chemical Biology, 2:548-552 (1998)).

Examples of polymer microsphere sustained release formulations are described in PCT publication WO 99/15154 (Tracy et al.), U.S. Pat. Nos. 5,674,534 and 5,716,644 (both to Zale et al.), PCT publication WO 96/40073 (Zale et al.), and PCT publication WO 00/38651 (Shah et al.). U.S. Pat. Nos. 5,674,534 and 5,716,644 and PCT publication WO 96/40073 describe a polymeric matrix containing particles of erythropoietin that are stabilized against aggregation with a salt.

In some embodiments, the STING inhibitor, STING agonist, cGAS inhibitor, or ENPP1 inhibitor are prepared with carriers that will protect the STING inhibitor, STING agonist, cGAS inhibitor, or ENPP1 inhibitor against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using known techniques. The materials can also be obtained commercially, e.g., from Alza Corporation and Nova Pharmaceuticals, Inc. Liposomal suspensions (including liposomes targeted to specific cells with monoclonal antibodies to cell-specific antigens) can also be used as pharmaceutically acceptable carriers. These can be prepared according to methods known to those skilled in the art, for example, as described in U.S. Pat. No. 4,522,811.

The STING inhibitor, STING agonist, cGAS inhibitor, or ENPP1 inhibitor can also be formulated to enhance intracellular delivery. For example, liposomal delivery systems are known in the art, see, e.g., Chonn and Cullis, “Recent Advances in Liposome Drug Delivery Systems,” Current Opinion in Biotechnology 6:698-708 (1995); Weiner, “Liposomes for Protein Delivery: Selecting Manufacture and Development Processes,” Immunomethods, 4(3):201-9 (1994); and Gregoriadis, “Engineering Liposomes for Drug Delivery: Progress and Problems,” Trends Biotechnol., 13(12):527-37 (1995). Mizguchi et al., Cancer Lett., 100:63-69 (1996), describes the use of fusogenic liposomes to deliver a protein to cells both in vivo and in vitro.

Dosage, toxicity, and therapeutic efficacy of the STING inhibitor, STING agonist, cGAS inhibitor, or ENPP1 inhibitor can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. In some embodiments, the STING inhibitor, STING agonist, cGAS inhibitor, or ENPP1 inhibitor exhibit high therapeutic indices. While the STING inhibitor, STING agonist, cGAS inhibitor, or ENPP1 inhibitor that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies within a range of circulating concentrations that include the ED50 with little or no toxicity. The dosage may vary within this range depending upon the dosage form employed and the route of administration utilized. For any STING inhibitor, STING agonist, cGAS inhibitor, or ENPP1 inhibitor, the therapeutically effective dose can be estimated initially from cell culture assays. A dose can be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

Typically, an effective amount of the STING inhibitor, STING agonist, cGAS inhibitor, or ENPP1 inhibitor, sufficient for achieving a therapeutic or prophylactic effect, ranges from about 0.000001 mg per kilogram body weight per day to about 10,000 mg per kilogram body weight per day. Suitably, the dosage ranges are from about 0.0001 mg per kilogram body weight per day to about 100 mg per kilogram body weight per day. For example, dosages can be 1 mg/kg body weight or 10 mg/kg body weight every day, every two days or every three days or within the range of 1-10 mg/kg every week, every two weeks or every three weeks. In one embodiment, a single dosage of a STING inhibitor, STING agonist, cGAS inhibitor, or ENPP1 inhibitor composition ranges from 0.001-10,000 micrograms per kg body weight. In one embodiment, the STING inhibitor, STING agonist, cGAS inhibitor, or ENPP1 inhibitor concentrations is in a carrier range from 0.2 to 2000 micrograms per delivered milliliter. An exemplary treatment regime entails administration once per day or once a week. In therapeutic applications, a relatively high dosage at relatively short intervals is sometimes required until progression of the disease is reduced or terminated, and until the subject shows partial or complete amelioration of symptoms of disease. Thereafter, the patient can be administered a prophylactic regime.

In some embodiments, a therapeutically effective amount of a STING inhibitor, STING agonist, cGAS inhibitor, or ENPP1 inhibitor may be defined as a concentration of a STING inhibitor, STING agonist, cGAS inhibitor, or ENPP1 inhibitor at the target tissue of 10⁻¹² to 10⁻⁶ molar, e.g., approximately 10⁻⁷ molar. This concentration may be delivered by systemic doses of 0.001 to 100 mg/kg or equivalent dose by body surface area. The schedule of doses would be optimized to maintain the therapeutic concentration at the target tissue. In some embodiments, the doses are administered by single daily or weekly administration, but may also include continuous administration (e.g., parenteral infusion or transdermal application). In some embodiments, the dosage of the STING inhibitor, STING agonist, cGAS inhibitor, or ENPP1 inhibitor compositions of the present technology is provided at a “low,” “mid,” or “high” dose level. In one embodiment, the low dose is provided from about 0.0001 to about 0.5 mg/kg/h, suitably from about 0.001 to about 0.1 mg/kg/h. In one embodiment, the mid-dose is provided from about 0.01 to about 1.0 mg/kg/h, suitably from about 0.01 to about 0.5 mg/kg/h. In one embodiment, the high dose is provided from about 0.5 to about 10 mg/kg/h, suitably from about 0.5 to about 2 mg/kg/h.

For example, when administered to the appropriate subject as determined by the methods of the present technology, a therapeutically effective amount of the STING inhibitor, STING agonist, cGAS inhibitor, or ENPP1 inhibitor may partially or completely alleviate one or more symptoms of cancer and/or lead to increased survival, decreased metastasis, reduced tumor burden, reduced tumor relapse during post-debulking adjuvant chemotherapy, reduction of the number of cancer cells, reduction of the tumor size, eradication of tumor, inhibition of cancer cell infiltration into peripheral organs, inhibition or stabilization of tumor growth, and stabilization or improvement of quality of life in the subject.

The skilled artisan will appreciate that certain factors may influence the dosage and timing required to effectively treat a subject, including but not limited to, the severity of the disease or disorder, previous treatments, the general health and/or age of the subject, and other diseases present. Moreover, treatment of a subject with a therapeutically effective amount of the therapeutic compositions described herein can include a single treatment or a series of treatments.

The mammal treated in accordance present methods can be any mammal, including, for example, farm animals, such as sheep, pigs, cows, and horses; pet animals, such as dogs and cats; laboratory animals, such as rats, mice and rabbits. In some embodiments, the mammal is a human.

### EXAMPLES

The following examples are provided to further illustrate the methods of the present disclosure. These examples are illustrative only and are not intended to limit the scope of the disclosure in any way.

### Materials and Methods

Cell culture: 4T1, CT26, and B16F10 cells lines were purchased from the American Type Culture Collection (ATCC) and cultured in DMEM (B16F10) or RPMI (4T1) supplemented with 10% FBS and 2 mM L-glutamine in the presence of penicillin (50 U ml⁻¹) and streptomycin (50 μg ml⁻¹). All cells were found to be negative for mycoplasma upon repeated testing.

The generation of knockout and knockdown cell lines for STING Inhibition Study: Murine cancer cells with Cgas and STING (Tmem173)-knockout were generated by Cas9 ribonucleoprotein nucleofection using a Lonza 4D-Nucleofector and SF Cell line Kit. crRNA (IDT, Coralville, IA) sequences is listed in Table 1. Two to four guides were screened per target and knockout cell lines were confirmed using immunoblotting. Antibody information used in immunoblotting experiments is listed in Table 2. Stable knockdown of cGAS or STING in MDA-MB-231 and 4T1 cells was achieved using shRNAs in pRRL (SGEP) plasmids obtained from the MSKCC RNA Interference Core. Two to four distinct shRNA hairpins were screened per target. Targeted shRNA sequences are listed in Table 1.

TABLE 1
shRNA hairpins and crRNA guide sequences

| Gene | crRNA vs. | |
|---|---|---|
| target | shRNA | Catalog number |
| Cgas | crRNA | ACGCAAAGATATCTCGGAGG |
| Cgas | crRNA | GCGAGGGTCCAGGAAGGAAC |
| Cgas | shRNA | TTCATATTCAATTTTCTTCGAT |
| Cgas | shRNA | TTCATAATATTCTTGTAGCTCA |
| Tmem173 | crRNA | GAAGGCCAAACATCCAACTG |
| Tmem173 | crRNA | CTACATAACAACATGCTCAG |
| Tmem173 | shRNA | TATATAAAATCCTTTGGCTGGG |
| Tmem173 | shRNA | TTCTACATATATAAAATCCTTT |

TABLE 2
Antibodies used in immunoblots

| Antibodies against | Clone | Company | Catalog number |
|---|---|---|---|
| Mouse cGAS | D3O8O | Cell Signaling Technology | 31659 |
| β-actin | AC-15 | Abcam | ab6276 |
| STING | D2P2F | Cell Signaling Technology | 13647 |
| α-tubulin | DM1A | Sigma-Aldrich | T9026 |
| p-IRF3 (Ser-396) | 4D4G | Cell Signaling Technology | 29047 |
| IRF3 | D83B9 | Cell Signaling Technology | 4302 |
| p-p65 (Ser-536) | 93H1 | Cell Signaling Technology | 3033 |
| p65 (NF-κB) | C-20 | Santa Cruz Biotechnology | sc-372 |
| p-RelB (Ser-552) | D41B9 | Cell Signaling Technology | 5025 |
| RelB | Polyclonal | Cell Signaling Technology | 4954 |
| Cyclophilin B | Polyclonal | Thermo Fisher Scientific | TAB1002 |
| HRP-linked anti-IgG | | Cell Signaling Technology | 7074 |

cGAMP quantification: For intracellular and extracellular cGAMP quantification in cancer cell lines, cancer cells were seeded in 15 cm culture dishes. When culture plates were 80-90% confluent, media was changed to serum free phenol red free RPMI (Corning). Sixteen hours following media exchange, the conditioned media was removed and centrifuged at ≥600×g at 4° C. for 15 minutes. Supernatant was assayed directly. All the steps were performed on ice. Cells were washed with PBS twice then trypsinzed for 5 min at 37° C. and cells counts were measured. Cells were then centrifuged at ≥600×g at 4° C. for 15 minutes. Whole cell lysates were generated by lysing the cell pellet in LP2 lysis buffer (Tris HCl pH 7.7 20 mM, NaCl 100 mM, NaF10 mM, beta-glycerophosphate 20 mM, MgCl2 5 mM, Triton X-100 0.1% (v/v), Glycerol 500 (v/v)). The homogenate was then subjected to centrifugation at 10,000 g for 15 min CGAMP ELISA was performed according to manufacturer's protocol using DetectX® Direct 2′,3′-Cyclic GAMP Enzyme Immunoassay Kit (Arbor Assays, Ann Arbor, MI).

Interferon-β quantification: Tumor tissues were weighed and lysed by adding lysis buffer RIPA (1:10 w/v) along with clean metal bead for 5 mins in tissue homogenizer. Samples were centrifuged at 15,000 g for 10 min at 4° C. Protein concentration was measured and final protein concentration of 300 ug/ul was prepared for every sample. Measurements of IFN-b levels in tumor lysates were done by Eve Technologies.

Immunoblotting: Cells were pelleted and lysed using RIPA buffer. Protein concentration was determined using BCA protein assay and 20-30 μg total protein were loaded in each lane. Proteins were separated by gradient SDS-PAGE and transferred to PVDF or nitrocellulose membranes. See Table 2 for antibody information. Membranes were imaged using the LI-COR Odyssey software. Relative STING protein levels were quantified by measuring band intensities on immunoblots using Image J software, background was subtracted and normalized to loading control. To validate the effect of STING inhibitor (C176) by western blot analysis, cells were transfected with STING ligand (2′3′-cGAMP, 20 μg/ml) (Invitrogen, Carlsbad, CA) with C176 at indicated doses or DMSO control for the indicated times. Cells were washed with ice-cold PBS and harvested using radio-immunoprecipitation assay (RIPA) lysis buffer containing protease and phosphatase inhibitors (Roche, Basel, Switzerland). Cells were lysed with a probe sonicator for 20 s. Protein quantification was performed using Pierce™ Rapid Gold BCA Protein Assay Kit (Thermo Fisher Scientific, Waltham, MA). Proteins were reduced at 95° C. for 5 mins with NuPAGE™ LDS Sample Buffer (4×) containing NuPAGE™ Sample Reducing Agent (10×). Lysates (40 μg protein/well) were resolved on NuPAGE™ 4-12% Bis-Tris Protein Gels (Invitro-gen) and transferred to polyvinylidene difluoride (PVDF) membranes (Thermo Fisher Scientific, Waltham, MA) using a wet transfer system. Membranes were blocked for 1 h at room temperature in Tris buffer saline (TBS) containing 0.1% Tween 20 (TBS-T) with either 5% (w/v) dried skimmed milk powder (Sigma-Aldrich, St. Louis, MO) for total protein antibodies or 3% (w/v) BSA (Thermo Fisher Scientific, Waltham, MA) for phospho-antibodies. Primary antibodies used are listed in Table 2. Membranes were incubated with respective primary antibodies at 4° C. overnight. Membranes were washed 3 times with TBST for 5 mins prior to 1 h incubation with Anti-rabbit IgG, HRP-linked Antibody (#7074, Cell Signalling Technology, Danvers, MA) at room temperature. Protein signals were visualized with Luminata™ Immobilon Forte Western HRP substrate. Images were captured by ChemiDoc Imaging Systems (Bio-Rad, UK) and densitometries were determined by ImageJ.

Immunofluorescence microscopy: Cells were fixed with ice-cold (−30° C.) methanol for 15 min. Subsequently, cells were permeabilized using 1% triton for 4 min. See Table 3 for antibody information. TBS-BSA was used as a blocking agent during antibody staining. DAPI was added together with secondary antibodies. Cells were mounted with Prolong Diamond Anti-fade Mountant (Life Technologies, Carlsbad, CA, P36961).

TABLE 3
Antibodies used in immunofluorescence

| Antibodies against | Clone | Company | Catalog number |
|---|---|---|---|
| Mouse cGAS | D3O8O | Cell Signaling Technology | 31659 |
| Human cGAS | Polyclonal | Millipore Sigma | ABF124 |
| Human cGAS | Polyclonal | Sigma Aldrich | HPA031700 |
| Human cGAS | 1697CT136.65.30 | LSBio | LS-C757990 |
| Human centromere | | Antibodies Incorporated | 15-234-0001 |
| proteins | | | |
| STING | D2P2F | Cell Signaling Technology | 13647 |

Wound scratch assay for cell migration: Tumor cell lines B16F10, 4T1, and CT26 were seeded in 6 well plates. When cells reached>90% confluence they were treated with 0.5 μM mitomycin C (M4287, Sigma-Aldrich, St. Louis, MO) for 2 hours, after which a wound was formed using a sterile p200 pipette tip. Thereafter cells were washed once with fresh media, following by treatment with C176 at 1 μM (Probechem, China) or DMSO control in media containing 1% FBS. Images were obtained at 0, 4, 8, and 12 hours using Olympus CKX53 microscope at 4× magnification. The wound surface area was measured and quantified using ImageJ software (MRI Wound Healing tool-dev.mri.cnrs.fr/projects/imagej-macros/wiki/Wound_Healing_Tool).

xCELLigence assayfor cell invasion: Assessment of the impact of STING inhibition on cell invasion was conducted using the xCELLigence system CIM plates (ACEA Biosciences, San Diego, CA). The upper chamber was coated with 1 mg/ml of growth factor complete Matrigel (#356234, Corning, UK) made up in serum free media (SFM) and incubated at 37° C. for 4 hours to allow polymerisation. A total of 4×10⁴ cells in serum-free media were seeded into the upper chamber insert with C176 at 1 μM (Probechem, China) or DMSO control. The lower chamber was filled with complete media. The CIM plates were incubated at 1 hour at 37° C. and 5% C02 for cell attachment. Plates were then monitored using the xCELLigence system. Changes in impedance resulting from cell invasion to the underside of wells were recorded every 15 minutes and monitored for a total of 60 hours.

Cell proliferation assay: Cell proliferation was determined by seeding of 5×10³ B16F10, 4T1 or CT26 cells in 6 well plates in triplicate. Cells were treated with C176 at 1 μM (Probechem, China) or DMSO control. Thereafter cells were counted in triplicate at 12, 24, 36, and 48 hours using trypan blue staining and the EVE™ automated cell counter (NanoEnTek, South Korea).

H&E staining oflung metastases: Lungs were excised from euthanized mice and submerged in 4% PFA overnight at 4° C. and then were transferred to 70% ethanol. Tissue embedding, slide sectioning, and H&E staining were performed by the Molecular Cytology Core Facility at MSKCC.

Quantitative PCR for STING inhibition study: RNA was extracted from cells with Trizol (Invitrogen, Carlsbad, CA, #15596026). cDNA was synthesized using the RNA to cDNA EcoDry™ Premix (Double Primed) kit (Takara #639549). Real-time PCR was performed to measure the relative mRNA expression levels of ISGs and the control GAPDH using Luna® Universal qPCR Master Mix (NEB, IPSWICH, MA, M3003L). The qPCR reaction and analysis were performed on a QuantStudio 6 platform (Life technology, Carlsbad, CA). The sequence of primers for Ccl5 is 5′-GCTGCTTTGCCTACCTCTCC-3′ and 5′-TCGAGTGACAAACACGACTGC-3′. The sequence of primers for Cxcl10 is 5′-CCAAGTGCTGCCGTCATTTTC-3′ and 5′-GGCTCGCAGGGATGATTTCAA-3′, The sequence of primers for Isg15 is 5-A AAGAA-GCAGATTGCCCAGAA-3′ and 5′-TCTGCGTCAGAAAGACCTCA-3′. The sequence for primers for Gapdh is 5′-AGGTCGGTGTGAACGGATTTG-3′ and 5′-TGTAGACCATGTAGTTGAGGTCA-3′.

Animal metastasis studies: Animal experiments were performed in accordance with protocols approved by the MSKCC Institutional Animal Care and Use Committee. For survival experiments, power analysis indicated that 15 mice per group were sufficient to detect a difference at relative hazard ratios of <0.25 or >4.0 with 80% power and 95% confidence, given a median survival of 58 days in the control group and a total follow up period of 180 days also accounting for accidental animal death during procedures. There was no need to randomize animals. Investigators were not blinded to group allocation. For tail vein injections, 5×10⁴ 4T1, 2.5×10⁴ B16F10, or 10⁵ CT26 cells were injected into the tail vein of 6-7-week old BALB/c (4T1 and CT26) or C57BL/6 (B16F10) mice. Metastasis was primarily assessed through overall survival. Overall survival endpoint was met when the mice died or met the criteria for euthanasia under the IACUC protocol. Animals were censored when death was confirmed to have occurred due to metastasis-unrelated causes such as injury or death during injection, surgery, or anesthesia. Surface lung metastases were assessed at endpoint by direct visual examination after euthanasia at which points lungs were perfused and fixed in 4% paraformaldehyde (4T1 and B16F10 experiments) or stained using India-ink (CT26 experiments). Furthermore, lung metastasis after injection of 4T1 cells was qualitatively assessed using routine hematoxylin and eosin (H&E) staining. For orthotopic tumor implantation, 2.5×10⁵ 4T1 cells in 50 μl PBS were mixed 1:1 with Matrigel (BD Biosciences, San Jose, CA) and injected into the fourth mammary fat pad. Only one tumor was implanted per animal. Primary tumors were surgically excised on day 7 after implantation and metastatic dissemination was assessed by monitoring overall survival or on day 30 through quantification of surface lung metastases upon euthanasia. The length (L) and width (W) of the primary tumors were measured using calipers. Tumor size was calculated according to the following formula: L*W2/2. C176 was reconstituted with DMSO to concentration of 25 mg/mL for the treatment of B16F10 and CT26 tumors, and 75 mg/mL for the treatment of 4T1 tumors. Each mouse was injected intraperitoneally daily with 8 μL of C176 stock solution in 100 μL of corn oil every day. H-151 was reconstituted in DMSO with concentration of 100 mM. Each mouse was injected intraperitoneally with 7.5 μL of H-151 stock solution in 200 μL of PBS with 0.1% Tween −80.

cGAS staining in MDA-MB-231 cell pellets: dnMCAK-expressing MDA-MB-231 cells with control or cGAS shRNA were harvested from 15-cm dishes by cell scraper and washed with cold PBS. Cells were resuspended in 4% Paraformaldehyde in PBS and were incubated at room temperature for 10 min. After replacing the fixation buffer with fresh 4% Paraformaldehyde, cells were incubated at 4° C. overnight. The fixation buffer was then replaced by 70% ethanol. After embedding with low-melting agarose, cell pellets were processed to paraffin blocks and were sectioned by the Molecular Cytology Core Facility at MSKCC. Immunofluorescence staining with three different cGAS antibodies were performed individually as described before. Anti-cGAS antibody LS-757900 was applied with 1:200 dilution. Anti-cGAS antibodies ABF124 and HPA031700 were applied with concentration of 1 μg/ml.

Analysis of cGAS and STING protein expression in breast tumor samples: Primary analysis of cGAS and STING protein expression was performed on a tissue microarray (TMA) of 217 FFPE TNBC samples. Samples and follow up data were collected under MSKCC TRB approval. There were 3 cores per tumor sample. Of the 217 samples, 183 and 180 samples had sufficient material for adequate assessment of cGAS and STING staining levels, respectively. This included 179 samples with adequate staining and quality to simultaneously quantify both proteins. Detailed clinical characteristics and clinical follow-up data were previously reported by Tozbikian, G., et al., PLoS ONE 9: e114900 (2014), the totality of which is incorporated herein by reference. Immunohistochemistry for cGAS and STING was performed on the automated Discovery XT processor (Ventana Medical Systems, Oro Valley, AZ) by the Molecular Cytology Core Facility at MSKCC. Briefly, after deparaffinized and tumor tissue conditioning, the antigen was retrieved using standard CC1 (Ventana Medical Systems, Oro Valley, AZ). Following blockage with Background Buster (Innovex, Lincoln, RI), the slides were incubated with 1:100 diluted anti-STING antibody for 4 hr, and then incubated with the biotinylated secondary antibody for 30 minutes. The Streptavidin-HRP D (DABMap kit, Ventana Medical Systems, Oro Valley, AZ) and the Alexa Fluor™ 488 Tyramide SuperBoost™ Kit, streptavidin (Life Technologies, Carlsbad, CA, catalog #: B40932) were used to detect the signal according to the manufacturer instructions. Then similar procedure was applied to detect cGAS with 1:100 diluted anti-cGAS antibody and Alexa Fluor™ 594 Tyramide SuperBoost™ Kit, strep-tavidin (Life Technologies, Carlsbad, CA, catalog #: B40935). Then the slides were counterstained with hematoxylin and were mounted with Permount mounting medium. Slides of immunofluorescence and immunohistochemistry were scanned with Pan-noramic Flash 250 (3DHistech, Budapest, Hungary) with 20×/0.8 NA air objective by the Molecular Cytology Core Facility at MSKCC. cGAS and STING protein expression levels were assessed manually using scores of 0 (absent), 1 (weak), 2 (moderate) and 3 (strong). STING expression was assessed separately in the tumor and stromal compartment. cGAS was rarely localized to micronuclei in the stroma and therefore was primarily assessed in the tumor compartment. Distant metastasis-free survival data were collected by reviewing medical records available at MSKCC. Tumors were categorized as having low (negative or weak) or high (moderate or strong) cGAS or STING expression.

cGAS staining in mucosal melanoma samples: Immunofluorescence for cGAS was performed on the automated Discovery XT processor (Ventana Medical Systems, Oro Valley, AZ) by the Molecular Cytology Core Facility at MSKCC with similar protocol described as above. Tumor mutational burden (TMB) and fraction of genome altered (FNA) values were taken from the Memorial Sloan Kettering-Integrated Mutational Profiling of Actionable Cancer Target (MSK-IMPACT) panel as described by Cheng, D. T., et al., BMC Med Genomics 10, 33 (2017), the totality of which is incorporated herein by reference. TMB was defined as the number of mutations per megabase and FNA was defined as the fraction of the genome with annotated copy number alterations as recently described by Bielski, C. M., et al., Nat. Genet. 50: 1189-1195 (2018), the totality of which is incorporated herein by reference.

RNA sequencing analysis: B16-F10 WT and STING KO cells were pretreated with 1-μM C-176 or DMSO for 48 hours, and media with fresh drug was added at 24 hours. RNA was extracted using the RNeasy Mini Kit (Qiagen, Hilden, Germany, 74104). Non-strand-specific paired end sequencing libraries were generated with TruSeq Stranded mRNA (Illumina, San Diego, CA, 20020594) and sequenced on the Illumina NovaSeq platform. Reads were mapped to the mouse reference GRCm38 with the Broad Picard Pipeline (broadinstitute.github.io/picard/). Gene expression level was estimated with GenomicAlignments (v1.18.1) as described by Lawrence, M., et al., PLoS Comput. Biol. 9, e10031182013 (2013), the totality of which is incorporated herein by reference. Differential analysis was performed by DESeq2 (v1.24.0) as described by Love, M. I., et al., Genome Biol. 15: 550 (2014), the totality of which is incorporated herein by reference. Gene set enrichment analysis was performed on the normalized reads estimated by DESeq2. Genes downregulated in C176-treated were filtered by two cutoffs: adjusted p value less than 0.05 and log 2-transformed fold change (C176 versus vehicle) less than −1. Genes downregulated in STING KO were filtered by two cutoffs: adjusted p value less than 0.1 and log 2-transformed fold change less than −1.

Data Availability: RNA sequence data used in this manuscript has been deposited at the Gene Expression Omnibus (GEO) accession number GSE148291 (www.ncbi.nlm.nih.gov/geo/query/acc.cgi?acc=GSE148291) and will be made publicly available upon publication of the manuscript. Reviewers can access the data using the following token: orwx-eywebjcrbmr. No new code was used in this manuscript.

The generation ofknockout cell lines ENPP1 Inhibition Study: Murine cancer cells deficient in Cgas, or Enpp1 were generated by Cas9 ribonucleoprotein nucleofection using a Lonza 4D-Nucleofector and SF Cell line Kit. CrRNA (IDT, Coralvile, IA) sequences is listed in Table 4. Four guides were screened per target and knockout cell lines were confirmed using immunoblotting. Antibody information used in immunoblotting experiments is listed in Table 5. Stable knockdown of ENPP1 in MDA-MB-231 cells was achieved using shRNAs in pRRL (SGEP or SGEN) plasmids obtained from the MSKCC RNA Interference Core. Four distinct shRNA hairpins were screened per target. Targeted shRNA sequences are listed in Table 4.

TABLE 4
crRNA guide sequences

| Gene | crRNA vs. | |
|---|---|---|
| target | shRNA | Catalog number |
| Enpp1 | crRNA | TACAACGCAAGTIGCCACTG |
| Enpp1 | crRNA | GATTCCGGATAAAGTCCCTA |
| Enpp1 | crRNA | GGTGACCGCTAATCATCAGG |
| Enpp1 | crRNA | GATTACCGTGATCTGAAATG |
| Enpp1 | crRNA | GAAGTCTATAACTTAATGTG |
| Cgas | crRNA | ACGCAAAGATATCTCGGAGG |
| Cgas | CrRNA | GCGAGGGTCCAGGAAGGAAC |
| ENPP1 | shRNA | TTAATAATCTTCTCTTCTGCCA |
| ENPP1 | shRNA | TTTCAATAAAAAATCATTCCAG |
| ENPP1 | shRNA | TTAGAGACAATTATATTCCGTA |
| ENPP1 | shRNA | TATTAAATAATTTTGAGTTGTA |

TABLE 5
Antibodies used in immunoblots

| Antibodies against | Company | Catalog number |
|---|---|---|
| mouse cGAS | Cell Signaling Technology | 31659 |
| β-actin | Abcam | ab6276 |
| STING | Cell Signaling Technology | 13647 |
| α-tubulin | Sigma-Aldrich | T9026 |
| Lamin B1 | Abcam | ab16048 |
| human ENPP1 | Abcam | ab223268 |
| human ENPP1 | Abcam | ab40003 |

Immunofluorescence microscopy: Cells were fixed with ice-cold (−30° C.) methanol for 15 min (when staining for centromeres and cGAS) or 4% paraformaldehyde (when staining for ENPP1 and GFP). Subsequently, cells were permeabilized using 1% triton for 4 min. See Table 6 for antibody information. TBS-BSA was used as a blocking agent during antibody staining. DAPI was added together with secondary antibodies. Cells were mounted with Prolong Diamond Antifade Mountant (Life Technologies, Carlsbad, CA, P36961).

TABLE 6
Antibodies used in immunofluorescence

| Antibodies against | Company | Catalog number |
|---|---|---|
| human ENPP1 | Abcam | ab223268 |
| human centromere | Antibodies Incorporated | 15-234-0001 |
| proteins | | |
| mouse cGAS | Cell Signaling Technology | 31659 |
| GFP | Sigma-Aldrich | 11814460001 |

ENPP1 staining of human xenografts: Immunohistochemistry for ENPP1 in human breast cancer xenografts was performed on the automated Discovery XT processor (Ventana Medical Systems, Oro Valley, AZ) by the Molecular Cytology Core Facility at MSKCC. Briefly, after deparaffinized and tumor tissue conditioning, the antigen was retrieved using sodium citrate pH6 buffer for 30 min. Following blockage with Background Buster (Innovex, Lincoln, RI), the slides were incubated with 2.5 μg/ml anti-ENPP1 antibody (Abcam ab4003 at 1:200, Table 7) for 4 hr, and then incubated with the biotinylated secondary antibody for 30 minutes. The Streptavidin-HRP D (DABMap kit, Ventana Medical Systems, Oro Valley, AZ) and the DAB detection kit (Ventana Medical Systems, Oro Valley, AZ) were used to detect the signal according to the manufacturer instructions. Then the slides were counterstained with hematoxylin and were mounted with Permount mounting medium. Tumor necrosis was assessed semi quantitatively by a certified pathologist based on the cross-sectional area containing necrosis. The pathologist was blinded to tumor group allocation.

TABLE 7
Antibodies used in immunohistochemistry

| Antibodies against | Company | Catalog number |
|---|---|---|
| human ENPP1 | Abcam | ab40003 |
| human ENPP1 | Abcam | ab223268 |
| CD45 | BD Pharmingen | 550539 |
| CD8α | Cell Signaling Technology | 98941 |
| NK1.1 | Thermo Fisher Scientific | MA1-70100 |
| human cGAS | LifeSpan BioSciences | LS-C757990 |
| Melan-A | Santa Cruz Biotechnology | sc-20032 |

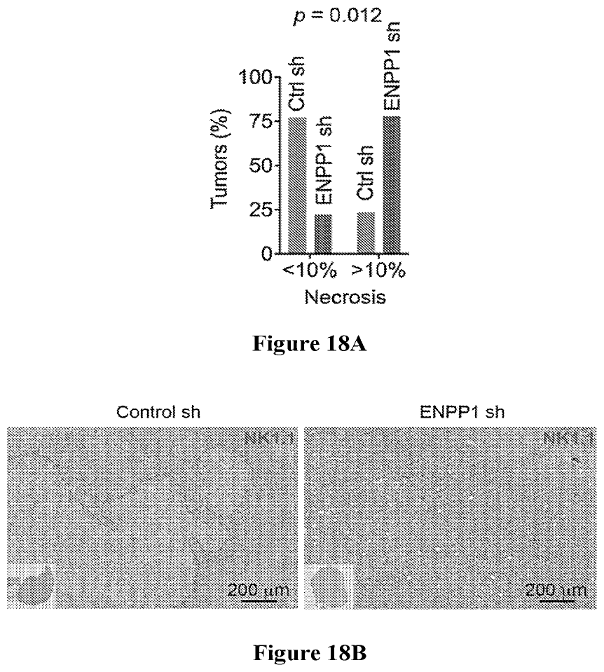

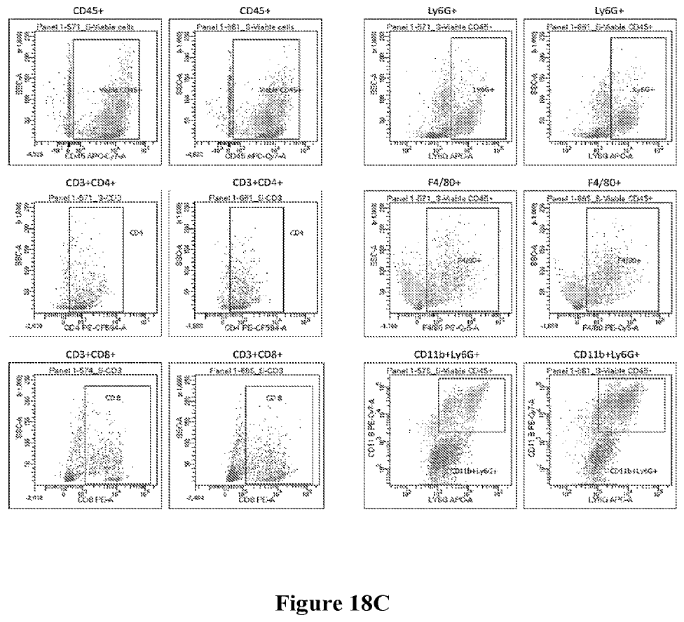

H&E staining and Immune phenotyping of lung metastases. Lungs were excised from euthanized mice and submerged in 4% PFA overnight at 4° C. and then were transferred to 70% ethanol. Tissue embedding, slide sectioning, and H&E staining were performed by the Molecular Cytology Core Facility at MSKCC. Immunohistochemistry for CD8 and CD45 staining were performed using anti-CD8 (#98941, Cell Signaling Technology, Danvers, MA) and anti-CD45 (Biosciences 550539) by the Laboratory of Comparative Pathology at MSKCC. For immune profiling using flow cytometry, animals were sacrificed 18 days after tail vein injection with control and ENPP1 KO 4T1 cells. Lungs were perfused through the right ventricle with 10-15 ml of PBS. The lungs were removed, and the large airways, thymus, lymph nodes were dissected from the peripheral lung tissue. The peripheral lung tissue was minced and transferred into 50 ml falcon tubes and processed in digestion buffer by mouse tumor dissociation kit (Miltenyi Biotec, Bergisch Gladbach, Germany), according to the manufacturer's instructions. Homogenized lungs were passed through 40-μm nylon mesh to obtain a single-cell suspension. The remaining red blood cells were lysed using BD Pharm Lyse (BD Biosciences, San Jose, CA). Cells were stained with viability dye LIVE/DEAD™ Fixable Blue Dead Cell Stain Kit (Invitrogen, Carlsbad, CA), followed by incubation with FcBlock (Invitrogen, Carlsbad, CA), and stained with a mixture of fluorochrome-conjugated antibodies (see Table 8 for a list of antibodies, clones, fluorochromes, and manufacturers). Data were acquired on a BD LSR II flow cytometer using BD FACS Diva software (BD Biosciences, San Jose, CA); compensation and data analysis were performed using FCS express 7 software. Unstained biological controls and single-color controls were used. Cell populations were identified using sequential gating strategy (FIG. 18C).

TABLE 8
Antibodies used in flow cytometry

| Antibodies against | Company | Catalog number |
|---|---|---|
| CD45, APC-eFluor 780 | Thermo Fisher Scientific | 47-0451-82 |
| Ly6G, APC | Thermo Fisher Scientific | 17-9668-82 |
| CD4, PE-Texas Red | Thermo Fisher Scientific | MCD0417 |
| F4/80, PE/Cy5 | BioLegend | 123112 |
| CD8, PE | Tonbo Biosciences | 50-0081-U500 |
| CD11b, PE/Cy7 | Thermo Fisher Scientific | 25-0112-82 |
| CD3c, BV785 | BioLegend | 100355 |
| PD1, APC/Cy7 | BioLegend | 135224 |

Quantitative PCR for Enp1 inhibition: RNA was extracted from cells with Trizol (Invitrogen, Carlsbad, CA, #15596026). CDNA was synthesized using the RNA to cDNA EcoDry™ Premix (Double Primed) kit (Takara #639549). Real-time PCR was performed to measure the relative mRNA expression levels of ENPP1 and the control GAPDH using Luna® Universal qPCR Master Mix (NEB, IPSWICH, MA, M3003L). The qPCR reaction and analysis were performed on a QuantStudio 6 platform (Life technology, Carlsbad, CA). The sequence of primers for ENPP1 is 5′-CAGTTGACAATGCCTTTGGAATG-3′ and 5′-CACTCTATCACAGGAGGTCTGG-3′. The sequence for primers for GAPDH is 5′-AGGTCGGTGTGAACGGATTTG-3′ and 5′-TGTAGACCATGTAGTTGAGGTCA-3′.

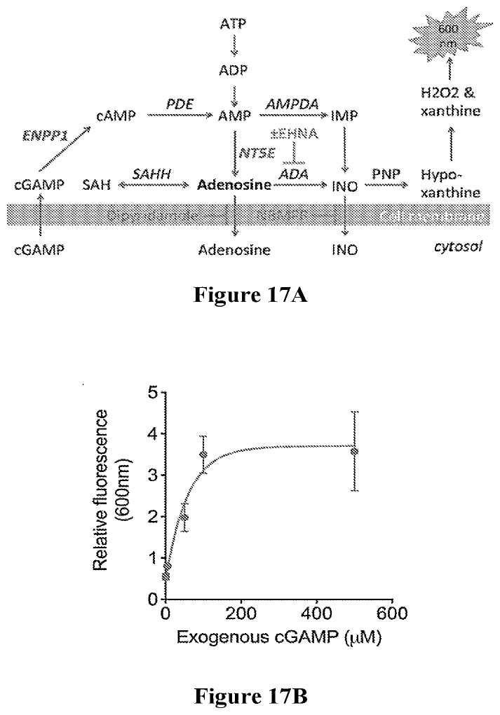

Adenosine measurements: 4T1 cells were seeded in 10 cm culture dishes in quadruplicates. When culture plates reached 80-90% confluence, 7 ml serum free phenol red free RPMI (Corning) with and without inhibitors (EHNA 100 μmol/L, NBMPR 100 μmol/L, Dipyridamole 40 μmol/L) was added to plates. Conditioned media was collected after 16 h incubation. Conditioned media was centrifuged at 10,000 g for 10 min at 4° C. Cells were harvested and cell counts were recorded for back calculations. Direct quantification of adenosine in flash-frozen conditioned media was performed by Charles River Laboratories Inc. (San Francisco, CA). Adenosine concentrations were determined by high performance liquid chromatography (HPLC) with tandem mass spectrometry (MS/MS) detection in multiple-reaction-monitoring mode (MRM). In brief, 4 μL of internal standard solution containing 10 nM Adenosine-13C5 was added to 10 μL of undiluted experimental sample. 10 μL was injected into an Infinity 1290 LC system (Agilent, USA) by an automated sample injector (SIL-20AD, Shimadzu, Japan). Analytes were separated by liquid chromatography using a linear gradient of mobile phase B at a flow rate of 0.200 mL/min on a reversed phase Atlantis T3 C18 column (2.1*150 mm, 3.0 μm particle size; Waters, USA) held at a temperature of 40° C. Mobile phase A consisted of 5 mM ammonium formate in ultrapure water. Mobile phase B was Methanol. Acquisitions were achieved in the positive ionization mode using a QTrap 5500 (Applied Biosystems, Foster City, CA) equipped with a Turbo Ion Spray interface. The ion spray voltage is set at 5.0 kV and the probe temperature is 500° C. The collision gas (nitrogen) pressure was kept at the Medium setting level. The following MRM transitions were used for quantification: m z 268.2/136.1 for Adenosine. Data were calibrated and quantified using the Analyst™ data system (Applied Biosystems, Foster City, CA, version 1.5.2). For indirect adenosine measurements in conditioned media after cGAMP addition were performed using the adenosine assay kit (Cell Biolabs, San Diego, CA) according to a modified manufacturer's protocol: for each sample, fluorescence intensity at 600 nm were measured with and without the adenosine deaminase inhibitor, EHNA (FIG. 17A).

Cellular growth and migration assays: Cellular proliferation rates were assessed by seeding 5×10⁴ control or Enpp1-KO 4T1 cells in 6-well plates (3-4 replicates per condition). Cells were seeded in the regular RPMI medium with 10% Fetal bovine serum (FBS). About 48 hours before cells growing to form a 90% confluency monolayer, regular media were replaced with media containing indicated drugs. The working concentration of cGAMP, adenosine, and the A2B antagonist PSB115 was 5.5 μm, 5.5 μm, and 1 μm, respectively. Fresh medium was changed every 12 hours. When reaching ˜90% confluency, cells were treated with RPMI medium containing 10 μm Mitomycin C for 1 hour. Wounds were formed using sterile P200 pipette tips. Images of the wounds were captured every 8 hours and were analyzed with a wound healing tool macro in ImageJ (dev.mri.cnrs.fr/projects/imagej-macros/wiki/Wound_Healing_Tool).

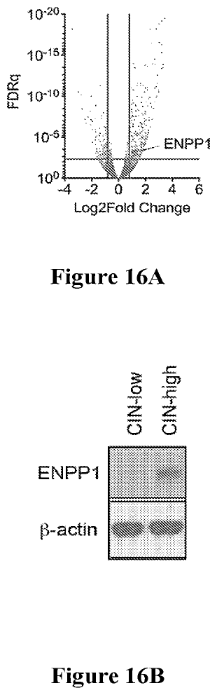

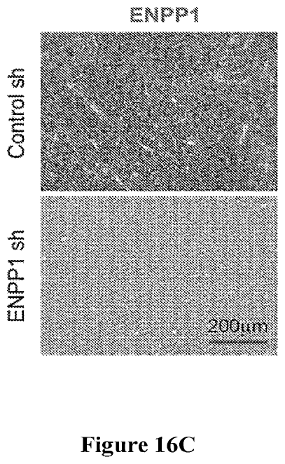

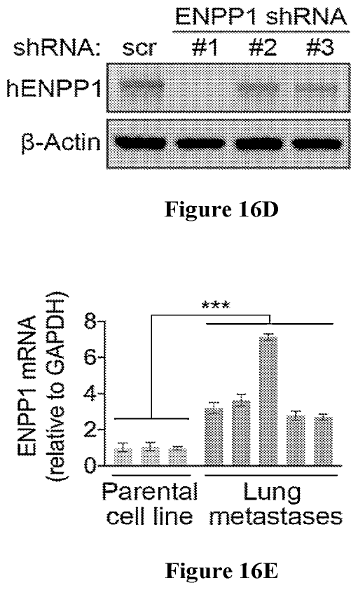

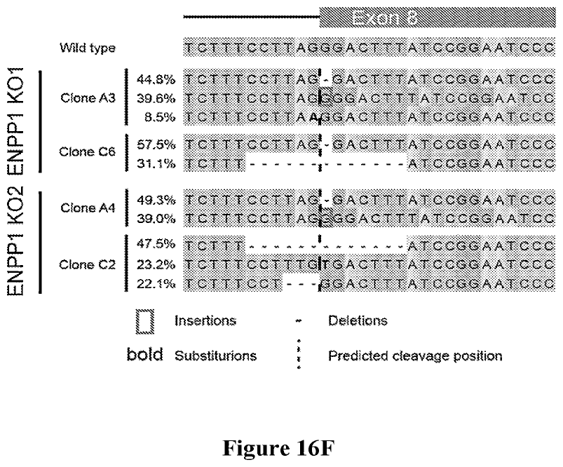

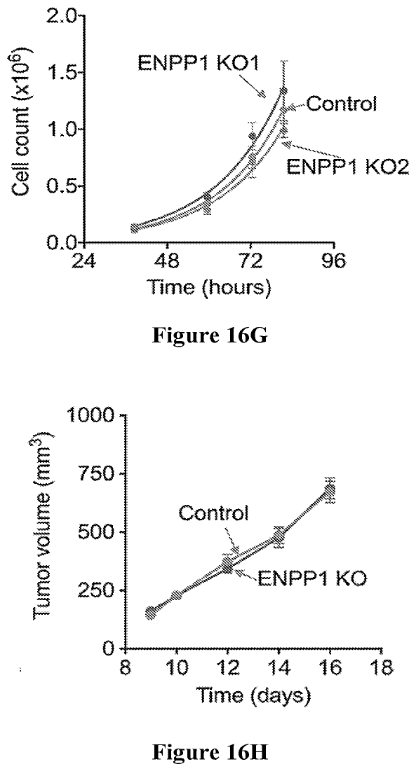

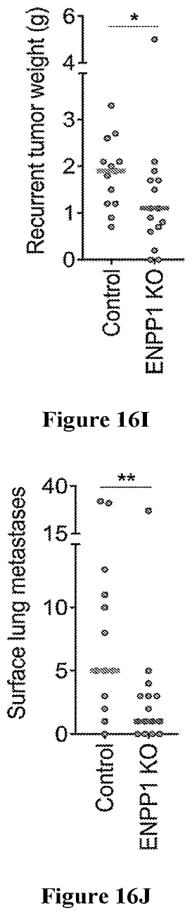

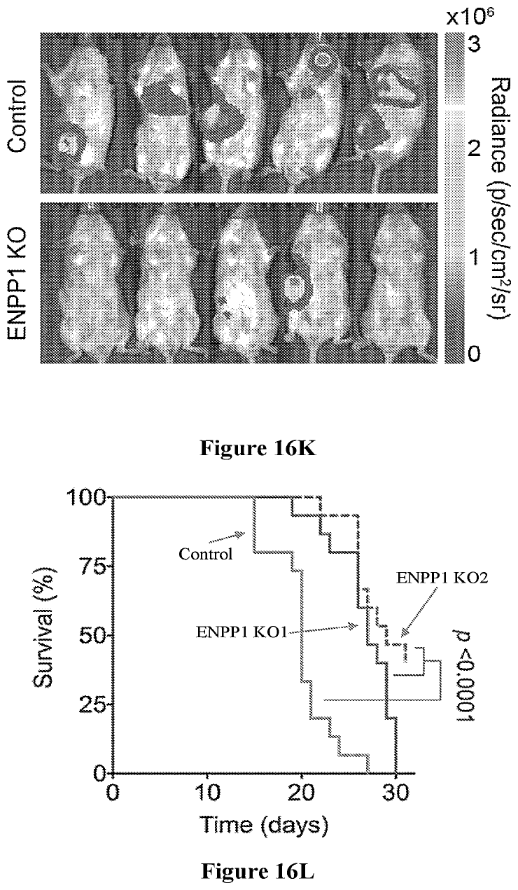

Animal metastasis studies: Animal experiments were performed in accordance with protocols approved by the MSKCC Institutional Animal Care and Use Committee. For survival experiments in 4T1 experiments, power analysis indicated that 15 mice per group would be sufficient to detect a difference at relative hazard ratios of <0.25 or >4.0 with 80% power and 95% confidence, given a median survival of 58 days in the control group and a total follow up period of 180 days also accounting for accidental animal death during procedures. There was no need to randomize animals. Investigators were not blinded to group allocation. For tail vein injections, 2.5×10⁴ 4T1 or 10⁵ CT26 cells were injected into the tail vein of 6-7-week old BALB/c mice. Metastasis was primarily assessed through overall survival. Overall survival endpoint was met when the mice died or met the criteria for euthanasia under the IACUC protocol. Surface lung metastases were assessed at endpoint by direct visual examination after euthanasia at which points lungs were perfused and fixed in 4% paraformaldehyde (4T1 experiments) or stained using india-ink (CT26 experiments). Furthermore, lung metastasis after injection of 4T1 cells was qualitatively assessed using routine hematoxylin and eosin (H&E) staining as shown in FIG. 14A. Metastatic dissemination in FIG. 16K was determined using bioluminescence imaging. Mice were injected with d-luciferin (150 mg kg⁻¹) and subjected to bioluminescence imaging (BLI) using tan IVIS Spectrum Xenogen instrument (Caliper Life Sciences, Waltham, MA) to image locoregional recurrence as well as distant metastases. BLI images were analyzed using Living Image Software v.2.50. For orthotopic tumor implantation, 5×10⁵ 4T1 cells in 50 μl PBS were mixed 1:1 with Matrigel (BD Biosciences, San Jose, CA) and injected into the fourth mammary fat pad. Only one tumor was implanted per animal. Primary tumors were surgically excised on day 7 after implantation and metastatic dissemination was assessed by monitoring overall survival or on day 30 through quantification of surface lung metastases upon euthanasia.

Analysis of ENPP1 protein expression and tumor infiltrating lymphocytes in breast tumor samples: Primary analysis of ENPP1 protein expression was performed on a tissue microarray (TMA) of comprising 226 TNBC FFPE tumor samples of which 223 had sufficient material. Samples and follow up data for cohort 1 were collected MSKCC IRB approval. There were 3 cores per tumor sample. Detailed clinical characteristics and clinical follow-up data were previously reported by Tozbikian, G., et al., PLoS ONE 9: e114900 (2014), the totality of which is incorporated herein by reference. Immunohistochemistry for ENPP1 in breast cancer cohort 1 was performed on the automated Discovery XT processor (Ventana Medical Systems, Oro Valley, AZ) by the Molecular Cytology Core Facility at MSKCC. Briefly, after deparaffinized and tumor tissue conditioning, the antigen was retrieved using standard CC1 (Ventana Medical Systems, Oro Valley, AZ). Following blockage with Background Buster (Innovex, Lincoln, RI), the slides were incubated with 2.5 μg/ml anti-ENPP1 antibody for 4 hr, and then incubated with the biotinylated secondary antibody for 30 minutes. The Streptavidin-HRP D (DABMap kit, Ventana Medical Systems, Oro Valley, AZ) and the DAB detection kit (Ventana Medical Systems, Oro Valley, AZ) were used to detect the signal according to the manufacturer instructions. Then the slides were counterstained with hematoxylin and were mounted with Permount mounting medium. Slides of immunofluorescence and immunohistochemistry were scanned with Pannoramic Flash 250 (3DHistech, Budapest, Hungary) with 20×/0.8 NA air objective by the Molecular Cytology Core Facility at MSKCC. ENPP1 protein expression levels were performed by a board-certified breast pathologist who was blinded to other clinicopathological characteristics and outcome. ENPP1 protein expression levels were assessed manually using scores of 0 (absent), 1 (weak), 2 (moderate) and 3 (strong) for both stromal and tumor compartments. Given this analysis was performed on small core material, ENPP1 expression was considered when >1% of cells showed a given staining pattern. Distant metastasis-free survival data were collected by reviewing medical records available at MSKCC. TILs were scored according to the recommendations of the international TILs working group based on the original hematoxylin and eosin-stained sections corresponding to each of the tumors present in the TMA, as described by Salgado R, et al., Ann. Oncol.: 259-271 (2015), the totality of which is incorporated herein by reference. Tumors were stratified as having low (negative or weak) or high (moderate or strong) ENPP1 expression. Independent validation studies were performed on a tissue microarray of n=91 estrogen receptor (ER) negative (Cohort 2) and n=115 ER positive (Cohort 3) FFPE breast tumors identified by the Northern Ireland Biobank (NIB), previously described elsewhere (36-39). Resected tumors were available between 1998 and 2008, with long-term follow-up data (relapse-free and overall survival) collated via the Northern Ireland Cancer Registry. Immunohistochemistry (IHC) was performed on 4 μm sections for CD8 (NIB15-0168, Office for Research Ethics Committees Northern Ireland (ORECNI) 13-NI-0149) using C8/144B, M7103, Dako at 1:50 dilution after an ER2 20 minutes retrieval, and for ENPP1 (NIB19-0301, ORECNI 13-NI-0149) using EPR22262-72, ab24538, Abcam at 1:1000 dilution. Slides were scanned on an Aperio AT2 Digital scanner at 40×. CD8+ T cell infiltration was reported as CD8+ cell density per mm² based on the total number of cells in each core and determined using the open-source digital pathological analysis software QuPath v0.1.2 (40,41). Cores with <100 tumor cells were removed from analysis and multiple core data were averaged. Rigorous quality control steps were taken to remove necrosis or keratin, tissue folds and entrapped normal structures; this was confirmed by a second reviewer with frequent consultation following an established method. ENPP1 protein expression levels were assessed manually using scores of 0 (absent), 1 (weak), 2 (moderate) and 3 (strong) for both stromal and tumor compartments as described above. Both analyses were performed blinded to other clinicopathological characteristics and outcome. Survival analysis was restricted to tumors with low nodal disease burden (NO-1). For OS analysis, ER− tumors were stratified as either positive (n=59) or negative (n=15) for ENPP1 staining. Given increased expression of ENPP1 in ER+ tumors in general, tumors were stratified as either having low (negative, weak, or moderate, n=41) or high (strong, n=42) ENPP1 staining.

ENPP1 staining and immune profiling of mucosal melanoma samples: Immunofluorescence for ENPP1 and cGAS was performed on the automated Discovery XT processor (Ventana Medical Systems, Oro Valley, AZ) by the Molecular Cytology Core Facility at MSKCC (Pubmed: 25826597). The procedure of deparaffinization, cell condition, antigen retrieval, and nonspecific blockages was similar as described in the immunohistochemistry section above. Instead of DAB detection kit, Tyramide-Alexa Fluor 488 (Invitrogen, Carlsbad, CA,T20922) and Tyramide-Alexa Fluor 594 (Invitrogen, Carlsbad, CA,T20935) were used for signal detection. CGAS and ENPP1 staining were sequentially performed with 1:200 diluted anti-cGAS and 2.5 μg/ml of anti-ENPP1 antibodies as the primary antibody. DNA was stained with 5 μg/ml of DAPI in PBS for 10 minutes. Then the slides were mounted with Mowiol mounting medium.

RNAseg analysis of TCGA tumors: RNA-seq data for human tumor samples from TCGA patients were obtained from (//gdc.cancer.gov/about-data/publications/pancanatlas). The data is upper-quartile normalized RSEM for batch-corrected mRNA gene expression and is from 33 different cancer types. Overall leukocyte fractions and CIBERSORT immune fractions for the TCGA Breast Cancer (BRCA) patients were obtained from (gdc.cancer.gov/node/998). The absolute abundance of the CIBERSORT immune cell types was obtained by multiplying the leukocyte fraction by the CIBERSORT immune fractions. The expression values for ENNP1 and CGAS from the TCGA RNA-seq data were utilized to categorize tumors into the four groups ENPP1_{low}cGAS^{low}, ENPP^{high}cGAS^{low}, ENPPh^{low}CGAS^{high}, and ENPP1^{high}cGAS^{high}. The median expression value per cancer type was used to categorize tumors into ENPP1^{low} and ENPP1^{high} groups. Tumors with expression values less than or equal to the median for a given cancer type were considered ENNP1^{low}, while tumors with expression values above the median were considered ENPP1^{high}. The bottom tertile expression value per cancer type was used to categorize tumors into cGAS^{low} and cGAS^{high} groups. Tumors with expression values less than or equal to the bottom tertile (<33%) of ccGAS expression in a given cancer type were categorized as cGAS^{low}, while tumors with expression values greater than the bottom tertile (>33%) were categorized as cGAS^{high}. The Wilcoxon Rank-Sum test was used to comp compare the relative abundance of CIBERSORT immune cell types between different cGAS/ENPP1 expression subgroups. For pathway enrichment analysis, the DESeq2 R package was used to identify differentially expressed genes between the ENPP1^{low}cGAS^{high} and ENPP1^{high}CGAS^{high} groups within the TCGA BRCA cohort. The Gene Set Enrichment Assay (GSEA) method was used to perform a pathway enrichment analysis between the ENPP1^{low}CGAS^{high} and ENPP1^{high}cGAS^{high} groups. A pre-ranked gene list from DESeq2 was created and sorted by the following: sign of the log fold change *−log(adjusted p-value). The sorted pre-ranked list was run in GSEA with the Hallmark gene set database that was downloaded from the Molecular Signatures Database (MSigDB). Survival analysis across TCGA tumor types were performed using KMPlot (http://www.kmplot.com) using auto-selection for best cutoff between the 25th and 75th percentiles.

RNAseg analysis ofhuman sarcomas: Matched clinicopathological and RNA sequencing data for samples annotated as undifferentiated pleomorphic sarcoma (UPS, also known as malignant fibrous histiocytoma) were obtained from The Cancer Genome Atlas (TCGA) Genomic Data Commons Data Portal repository in May 2018. TCGA Samples were collected retrospectively from multiple institutions following institutional review board approval, processed, molecularly characterized, and pathologically verified by the TCGA Biospecimen Core Resource at the National Cancer Institute, as previously described by Cancer Genome Atlas Research Network, Cell 171: 950-965.e28 (2017), the totality of which is incorporated herein by reference. Raw read counts were utilized for our analysis. Two additional publicly available RNA sequencing datasets of UPS tumors were obtained for validation as described by Lesluyes T, et al., Eur J Cancer 57:104-111 (2016) and by Hoadley K A, et al., Cell: 173:291-6 (2018), respectively, the totality of which is incorporated herein by reference. For analysis of the dataset (EGAD00001004439) as described by Steele C. D., et al., Cancer Cell 35: 441-448 (2019), the totality of which is incorporated herein by reference, previously processed data (transcripts per million) were utilized. For analysis of the dataset as described by Lesluyes T., et al., Eur J Cancer 57:104-111 (2016), the totality of which is incorporated herein by reference, FASTQ files (SRA accession ID SRP057793) were preprocessed with Kallisto using the human genome reference GRCh38 and transcript level abundances were computed using the Bioconductor package tximport, as described by Soneson C., et al., F1000Res 4:1521 (2015), the totality of which is incorporated herein by reference. The abundance of tissue-infiltrating immune cells was estimated using transcriptome-based methods. The Microenvironment Cell Populations-counter (MCP-counter) method was used to determine relative abundance of various tumor immune microenvironment constituents. Specifically, MCP-counter cytotoxic T-lymphocyte (CTL) scores were calculated from expression of seven transcripts including CD8A and log 2-normalized. CTL scores were validated using an orthogonal transcriptome-based method, cytolytic activity (CYT) scores, calculated as the geometric mean of granzyme A (GZMA) and perforin (PRFI) transcript counts.

Bladder cancer response data to anti-PD-L1 treatment: RNA sequencing data was described by Mariathasan S., et al., Nature 554:544-548 (2018), the totality of which is incorporated herein by reference, a metastatic urothelial cancer anti-PD-L1 treated cohort in SRA format, and reverted back to FASTQ using bam2fastq (v1.1.0). FASTQ reads were aligned to the hg19 genome using STAR as described by Dobin A., et al., Bioinformatics 29: 15-21 (2013), the totality of which is incorporated herein by reference. Transcript quantification was performed using RSEM with default parameters described by Li B., et al., BMC Bioinformatics 12: 323 (2011), the totality of which is incorporated herein by reference. Response was defined based on radiological response as per the RECIST criteria, with “CR/PR” being classified as a responder and “SD/PD” being a non-responder. The cGAS^{high} group was defined as the upper two tertiles, and cGAS^{low} as the bottom tertile, of CGAS expression.

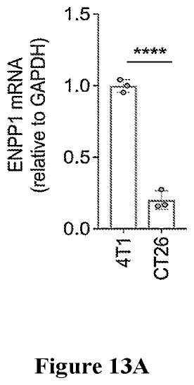

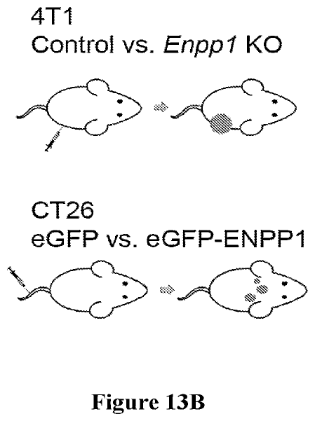

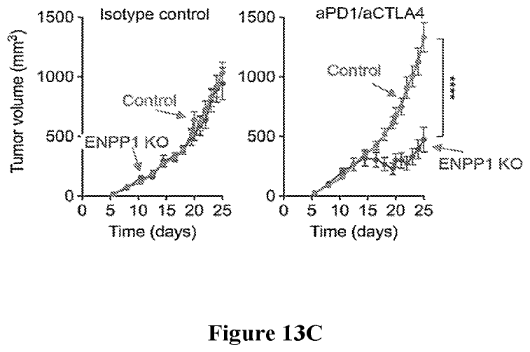

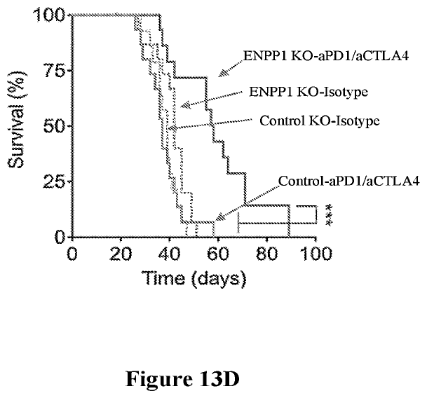

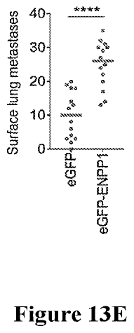

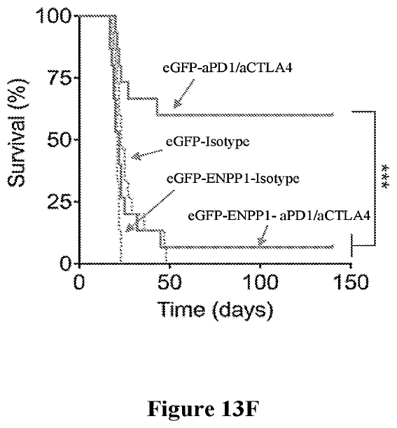

Animal immunotherapy experiments. To assess the role of ENPP1 in the primary tumor growth upon the immune checkpoint blockade (ICB), the 4T1 orthotopic mammary fat pad implantation model were adopted. First, 4T1 cells (4T1-Luc) cells and 4T1-Luc Enpp1 knockout (KO) cells were generated by stably integrating the Lentivirus pLVX vector expressing the tdTomato-Luciferase fusion gene in the 4T1 and 4T1 Enpp1-KO cells, respectively. Fifteen ˜7-week-old mice were used for each of the arm, including four combinations of two cell lines (4T1-Luc and 4T1-Luc ENPP1 KO) and two conditions (ICB and the isotype control treatment). 250,000 4T1-Luc cells or 4T1-Luc Enpp1-KO cells in PBS:Matrigel (1:1) mix were injected into the mammary fat pad of Balb/c mice. 200 μg rat anti-mouse PD1 IgG2a antibody (aPD1) and 100 μg mouse anti-mouse CTLA4 IgG2b antibody (aCTLA4) or their corresponding isotype control antibodies were delivered intraperitoneally in 100 ml of PBS to mice every 3 days starting at day 6 post implantation. After 4 doses of combined ICB, maintenance aCTLA4 treatment and the corresponding isotype control were given every 3 days. The length (L) and width (W) of the tumor were measured using calipers. The tumor size was calculated according to the following formula: L*W²/2. For the experiment in FIG. 13D, endpoint was determined when a primary tumor size of 2000 mm³ was seen. For the CT26 model, 100,000 eGFP or eGFP-ENPP1 expressing CT26 cells were delivered intravenously to 7 week-old Balb/c mice. Treatment with aPD1/aCTLA4 antibodies and their corresponding isotype control antibodies was initiated intraperitoneally starting on day 6 and given every 3 days for 5 total doses. Animals were monitored for overall survival.

### Example 1: Constitutive Activation of cGAS-STING Signaling in Chromosomally Unstable Cancer Cells Materials and Methods

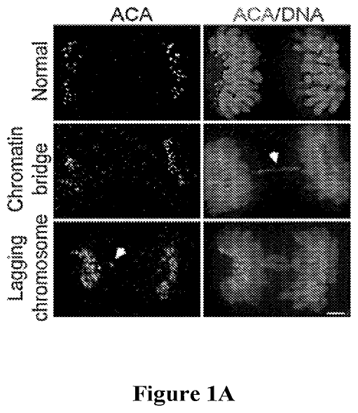

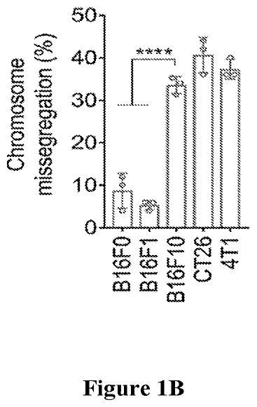

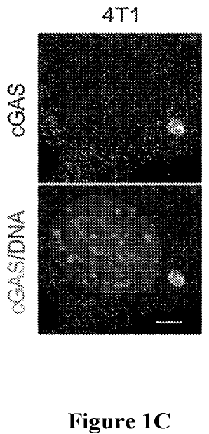

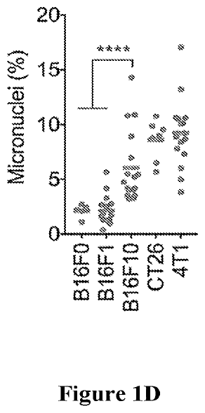

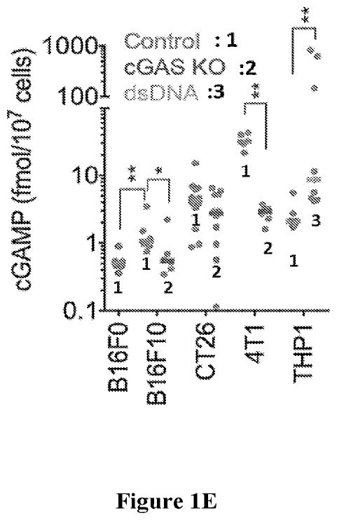

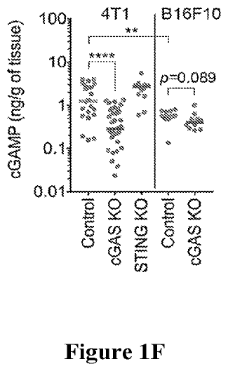

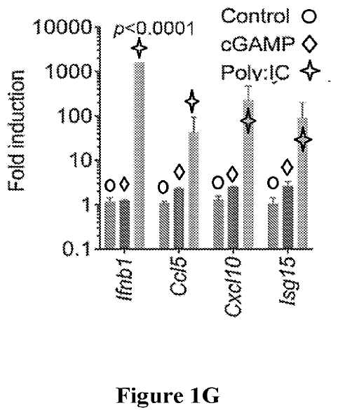

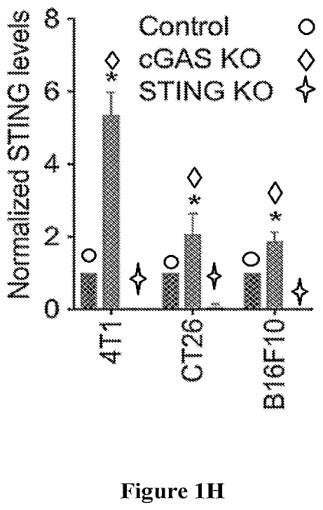

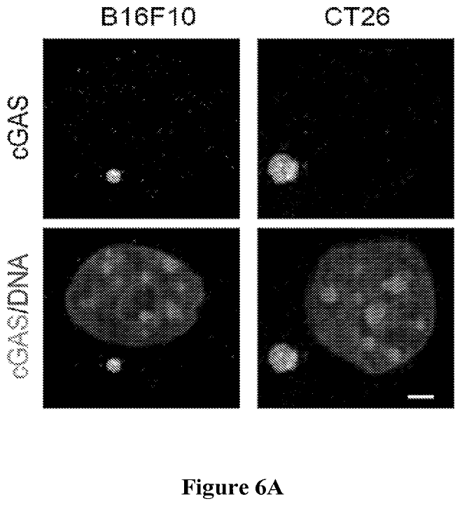

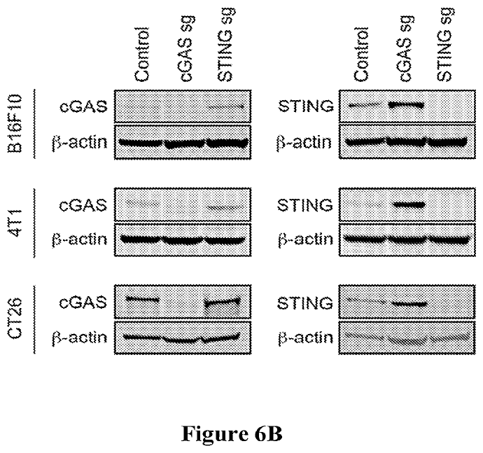

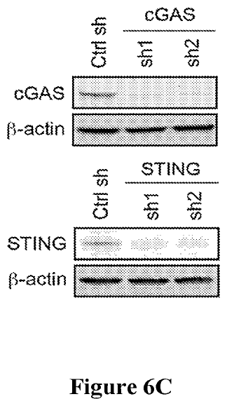

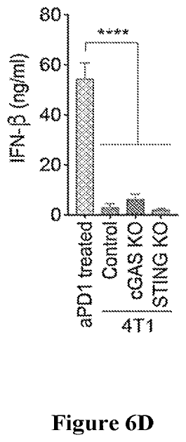

First the degree of chromosomal instability (CIN) in syngeneic mouse models of melanoma, with low (B16F0 and B16F1) or high lung-metastatic potential (B16F10), and highly metastatic models of colorectal (CT26) and triple-negative breast cancer (TNBC, 4T1), were assessed. Compared to B16F0 and B16F1, models with increased metastatic proclivity (B16F10, 4T1 and CT26) had significantly higher frequencies of chromosome missegregation during anaphase, a hallmark of CIN (FIGS. 1A-1B). These cells contained a preponderance of micronuclei characterized by cGAS localization, suggestive of cytosolic exposure of their enclosed genomic dsDNA (FIGS. 1C-1D and FIG. 6A). Next, cGAMP levels in cell lysates were measured using an ELI-SA-based method. All tested cell lines had detectable, but variable levels of cGAMP, which were significantly reduced upon CRISPR-Cas9 knockout (KO) of Cgas (FIGS. 1E and 6B). The levels of cGAMP correlated with the frequency of micronuclei and the metastatic potential of each cell line (FIG. 1E). Furthermore, basal cGAMP concentrations observed in 4T1 cells, characterized by the highest frequency of micronuclei, paralleled those observed in the monocytic cell line, THP1, upon acute transfection with dsDNA (FIG. 1E). This suggests that chromosomally unstable cancer cells can generate significant amounts of cGAMP at baseline as a result of constitutive cGAS activation.

In a tumor, cGAMP may derive from either cancer cells or host cells. To determine the source of cGAMP in vivo, B16F10 cells were subcutaneously implanted in C57BL/6 mice, and 4T1 cells were subcutaneously implanted into the mammary fat pad of BALB/c mice. Relative cGAMP levels recovered from total tumor lysates mirrored those observed in cell lines, with 4T1 tumors displaying higher concentrations of the cyclic dinucleotide relative to their B16F10 counterparts (FIG. 1F). Importantly, KO of cGAS—but not of STING (Tmem173)—in tumor cells led to a marked reduction in cGAMP recovered from total tumor lysates (FIG. 1F), suggesting that cancer cells produce a significant proportion of the total tumor cGAMP.

Despite constitutive cGAS-STING activation, 4T1 cells had low baseline expression of Ifnb1 and other ISGs. Furthermore, there was no induction in Ifnb1 and only a minimal increase in ISG expression (˜2-fold) upon cGAMP transfection (FIG. 1G). Notably, failure to induce a type I IFN response was specific to STING, as transfection with Poly(I.C), an activator of dsRNA sensing pathways, led to a robust induction of ISGs (FIG. 1G). Similar to our in vitro findings, control, cGAS-KO, and STING-KO tumors had low basal—and nearly undetectable—levels of IFN-β, when compared to those observed during active anti-tumor immune responses arising from immune checkpoint blockade of a responsive tumor model used as a reference control (FIG. 6D).

In comparison to IFN-competent normal cells, tumor cells generally have reduced STING protein levels. It is postulated that the lack of a robust IFN response was due to reduced STING signaling strength which dictates its output. Whether low STING levels in highly metastatic cells were the result of increased STING turnover and degradation, which results from its stimulation by cGAMP, was then investigated. Indeed, KO of cGAS in all three metastatic cancer cell lines led to a significant increase in STING protein levels (FIGS. 1H and 6B). Remarkably, loss of cGAS activation restored IFN-responsiveness in tumor cells as evidenced by marked induction of ISGs upon cGAMP transfection and this response was suppressed in the presence of the STING inhibitor, C-176 (FIG. 1I). Collectively, these data suggest that chromosomally unstable cancer cells exhibit chronic cGAS-STING signaling leading to increased turnover and low steady-state levels of STING protein, which are insufficient to generate a robust IFN response. 4T1, CT26, and B16F10 cells lines were purchased from the American Type Culture Collection (ATCC) and cultured

### Example 2: Chronically Active STING Promotes Metastasis in Immunocompetent Settings

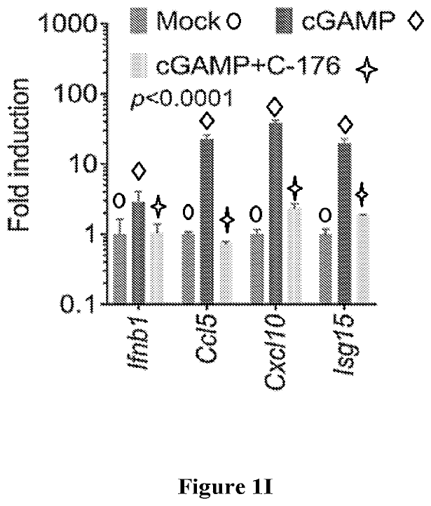

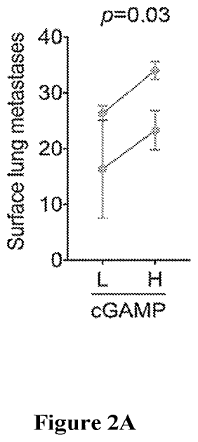

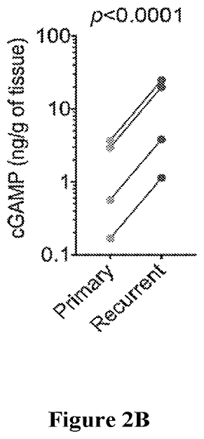

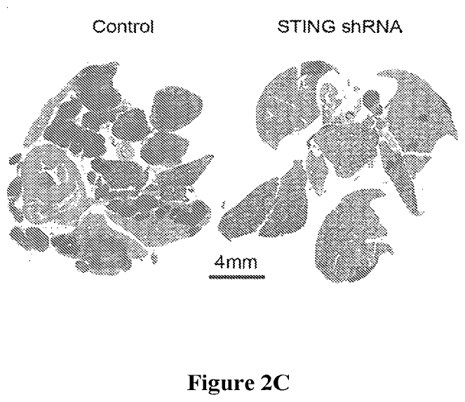

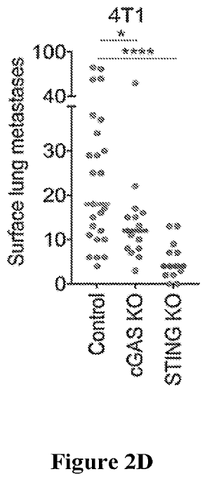

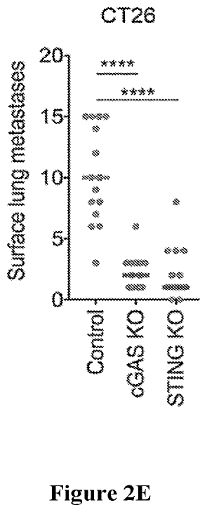

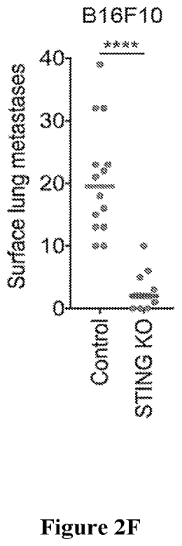

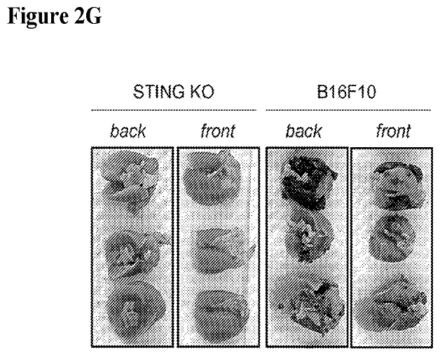

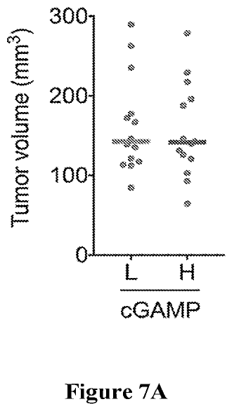

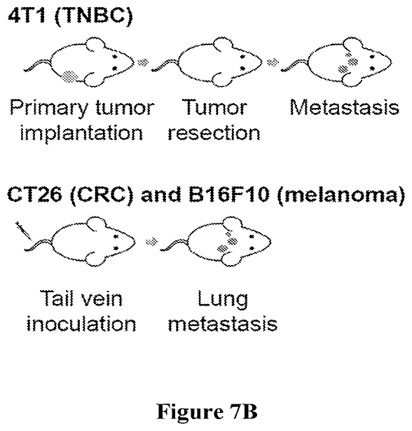

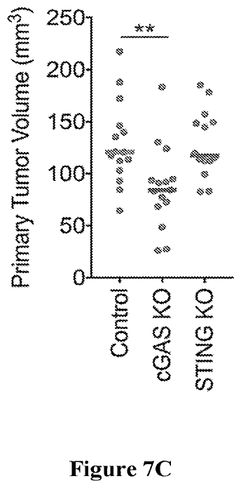

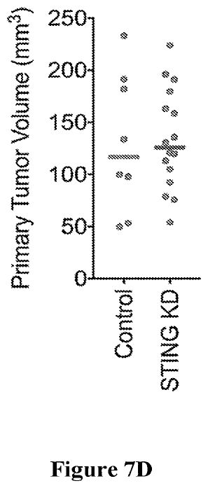

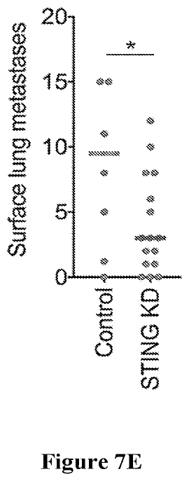

To evaluate the consequences of chronic cGAS-STING signaling in immunocompetent models, cGAMP concentrations in two independent cohorts of orthotopically transplanted 4T1 breast tumors at the time of resection were measured, and it was found that tumors with cGAMP levels above the median values portended the development of more numerous lung metastases (FIG. 2A). There was no association between relative cGAMP levels and primary tumor size (FIG. 7A). Furthermore, locally recurrent tumors had significantly more cGAMP when compared to their matched primary tumors (FIG. 2B).

Next, lung metastatic burden after orthotopic tumor transplantation followed by early resection (4T1) or as a result of direct tail vein inoculation of tumor cells (B16F10 and CT26) upon CRISPR-Cas KO or shRNA-mediated depletion of Cgas or STING (FIGS. 6B-6C and 7B) was assessed. Loss of STING did not impact primary tumor size, while Cgas KO tumors were smaller compared to the parental control tumors (FIGS. 7C-7D). On the other hand, loss of either cGAS or STING from tumor cells led to significant reduction in surface lung metastases in all three immuno-competent models tested (FIGS. 2C-2G and 7E). Therefore, while inadequate to induce and IFN response, persistent cGAMP-mediated STING activation in chromosomally unstable cancer cells can promote metastatic progression in immuno-competent settings.

### Example 3: Pharmacologic STING Inhibition Suppresses Cancer Cell Migration and Invasion

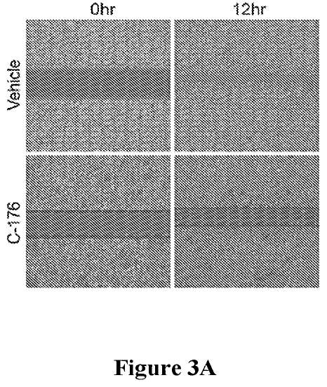

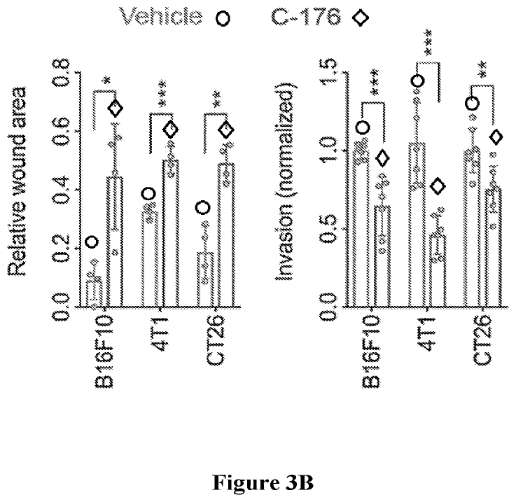

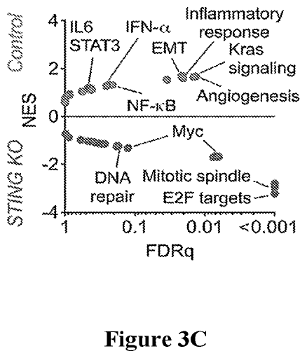

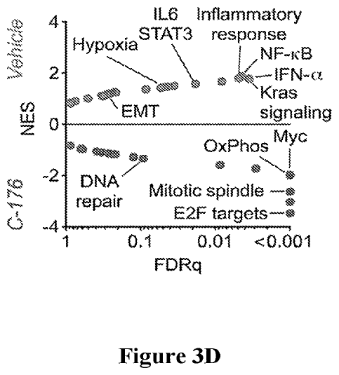

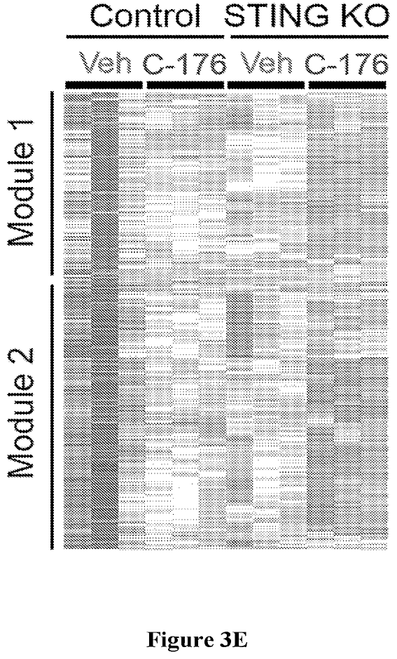

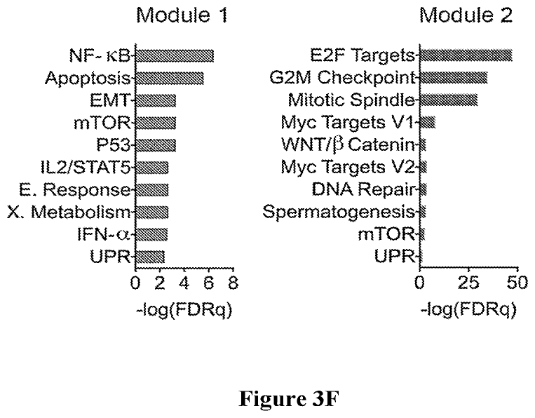

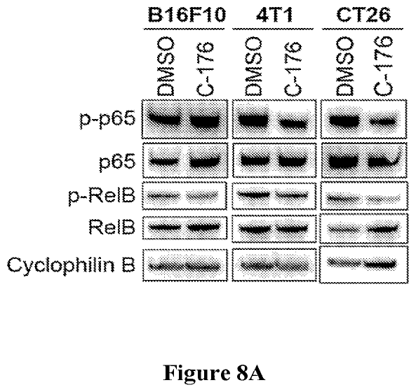

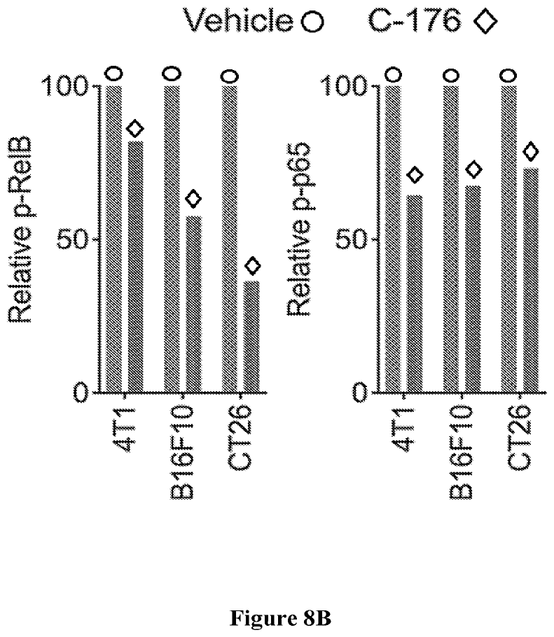

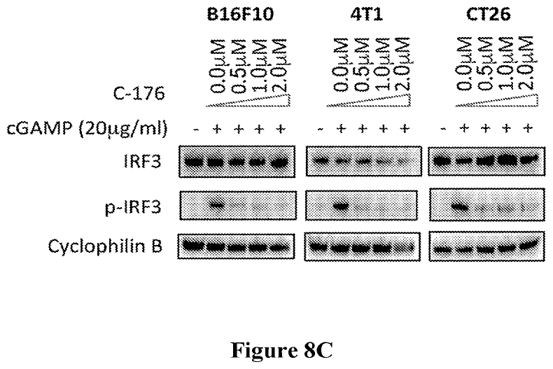

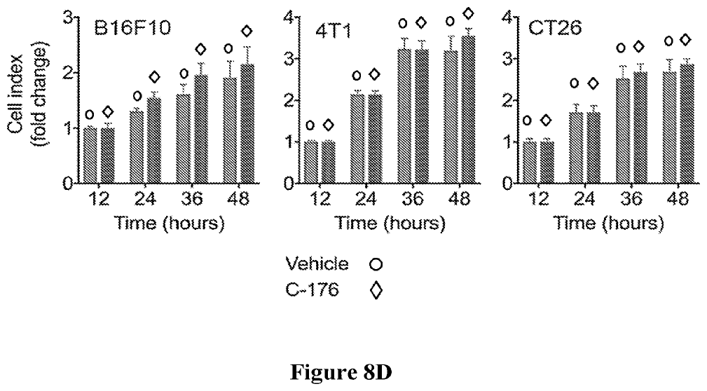

To assess whether pharmacologically targeting STING could phenocopy its genetic knockout, C-176, a covalent inhibitor that blocks activation-induced palmitoylation of the murine STING isoform, was used. C-176 inhibited baseline inflammatory signaling downstream of STING in tumor cells as evidenced by significant reductions in the phosphorylated pools of the NF-κB transcription factors, p65 and RelB (FIGS. 8A-8B). It also suppressed IRF3 phosphorylation upon transfection of large amounts (20 μg/ml) of cGAMP (FIG. 8C). At these inhibitory concentrations, C-176 did not impact cellular proliferation (FIG. 8D). Addition of C-176 to tumor cells led to significant reductions in both migration and invasion of all three metastatic cancer cell lines (FIG. 3A-3C).

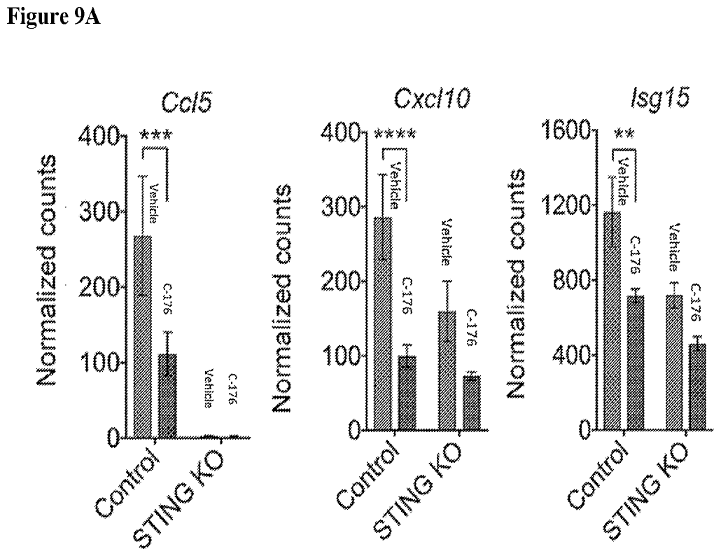

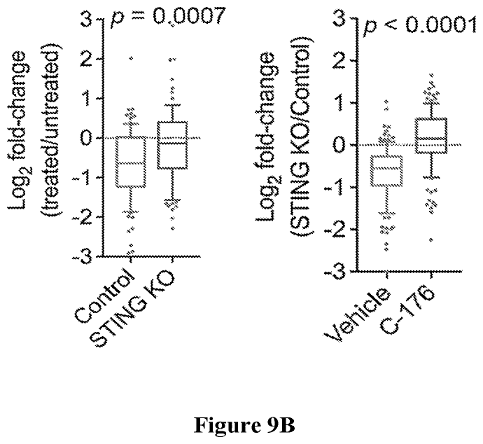

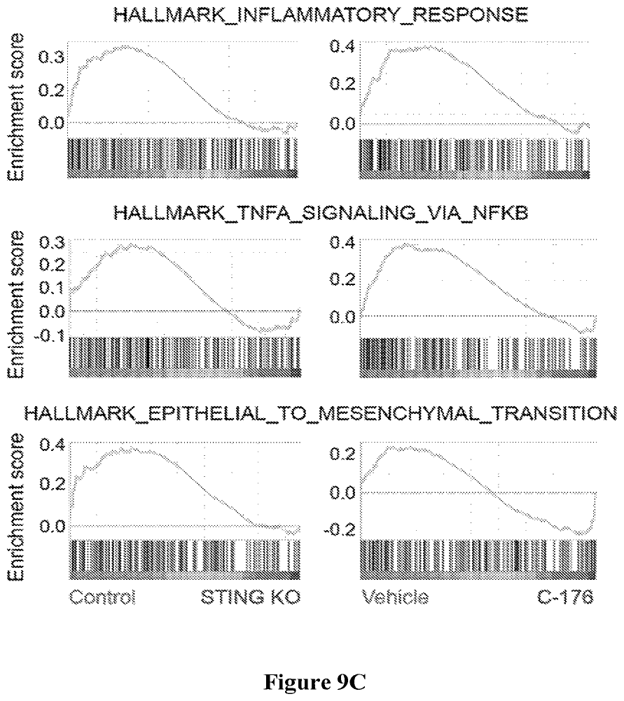

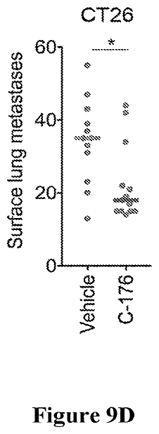

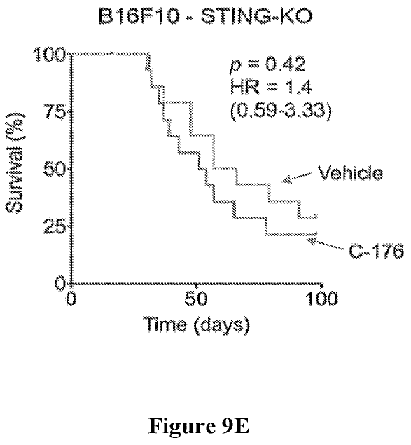

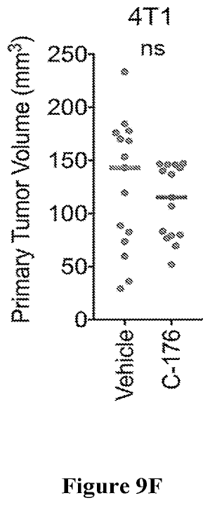

To further characterize the effects of STING inhibitors on tumor cells, RNA-sequencing of control and STING-KO B16F10 cells treated with C-176 or vehicle control was sequenced. Treatment with the STING inhibitor led to significant reductions in baseline expression of inflammation-related genes, such as Ccl5, Cxcl10, and Isg15 (FIG. 9A). Baseline expression of type I IFN genes was either minimal (Ifnb1) or completely undetectable (IFN-a genes), exhibiting no significant change between conditions (not shown), suggesting that baseline inflammation-related gene expression is likely IFN-independent. Using gene set enrichment analysis (GSEA), it is found that treatment with C-176 phenocopied STING KO, and either treatment led to downregulation of pathways related to inflammation and of genes involved in the epithelial-to-mesenchymal transition (EMT) (FIGS. 3C-3D and 9C). In line with on-target activity of the inhibitor, expression of STING-dependent genes was significantly reduced upon treatment with C-176 (FIG. 9B). Similarly, genes whose expression was reduced after C-176 treatment were also suppressed upon STING KO (FIG. 9B). Next 724 genes that were differentially expressed upon C-176 treatment in control, but not in STING KO cells were identified (FIG. 3E). These genes were divided into two modules, the first of which consisted of inflammation and EMT-related genes, and was upregulated in control cells and suppressed upon STING inhibition. The second module, which defined STING-inhibited states, encompassed pathways involved in cell cycle and metabolism (FIG. 3F). This dichotomy in transcriptional responses suggests that chronic cGAS-STING activation underlies baseline inflammatory and migratory cancer cell behaviors in a manner that is amenable to pharmacologic intervention.

### Example 4: STING Inhibitors Suppress Cancer Metastasis

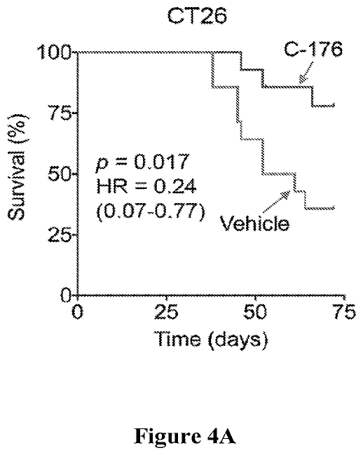

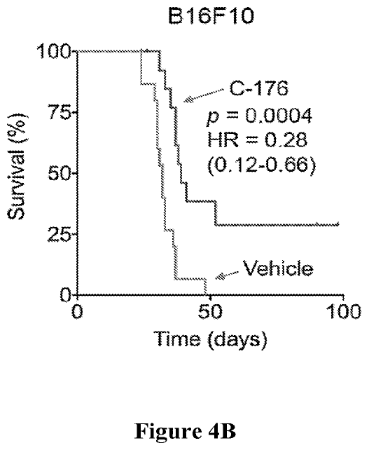

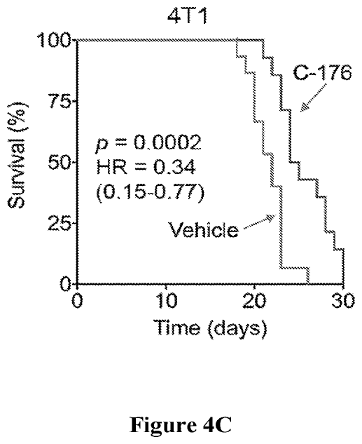

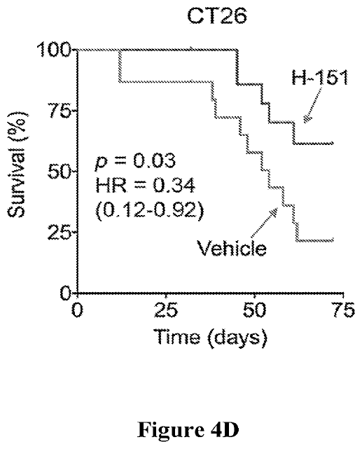

To test the effect of pharmacologic STING inhibition on cancer metastasis in immunocompetent settings, C-176 was delivered through daily IP injections to tumor bearing animals and both survival and the development of surface lung metastases after tail vein inoculation of tumor cells were monitored. Strikingly, C-176 led to a significant reduction in the number of CT26 lung metastasis 13 days after tail vein injection and significantly prolonged survival of tumor bearing mice in all three syngeneic models (FIGS. 4A-4C and 9D). In B16F10-bearing animals, nearly 30% of the treated group had no evidence of disease at 98 days (FIG. 4A). Furthermore, treatment of CT26 bearing animals with H-151, another STING inhibitor, led to significant prolongation in overall survival compared to vehicle-treated control animals (FIG. 4D). The efficacy of STING inhibition on metastasis mirrored the extent of reduction in RelB phosphorylation after C-176 treatment (FIGS. 4A-4C and 8B). To ensure that this therapeutic effect is mediated through cancer cell-intrinsic STING inhibition, C-176 was administered to C57BL/6 mice inoculated with STING-KO B16F10 cells. In these mice, C-176 treatment did not provide an additional survival advantage beyond STING KO (FIG. 9E). Thus, the therapeutic effect of the STING inhibitors on metastasis is primarily mediated through inhibition of cancer cell-intrinsic STING. Furthermore, the effect of pharmacologic STING inhibition was specific to metastatic progression, as it did not have a significant impact on 4T1 primary tumor size (FIG. 9F), mirroring genetic results. Notably, prolonged daily treatment with the STING inhibitor was well tolerated and did not lead to any clinically evident toxicity when compared to vehicle-treated control animals. Non-tumor-bearing animals treated with vehicle or C-176 for 87 days survived for the entire duration of treatment. End-of-treatment necropsy of C-176-treated animals revealed no distinct pathologic abnormalities compared to vehicle-treated animals. Furthermore, blood counts and blood chemistries were within normal limits, with the exception of lower baseline cortisol levels in C-176-treated animals, in line with its anti-inflammatory properties (Tables 9 and 10).

TABLE 9
Blood chemistry of mice treated with C-176 and vehicle control

| | C-176 | | Vehicle | | Reference |
|---|---|---|---|---|---|
| Animal | 1 | 2 | 1 | 2 | Range |
| [BUN (mg/dL)] | 17 | 16 | 18 | 16 | 5.0-28 |
| [CREA (mg/dL)] | 0.20 | 0.19 | 0.17 | 0.15 | 0.2-0.5 |
| BUN/CREA Ratio | 85.0 | 84.2 | 105.9 | 106.7 | — |
| [ALP (U/L)] | 61 | 40 | 101 | 75 | 105-370 |
| [ALT (U/L)] | 26 | 32 | 43 | 40 | 27-195 |
| [AST (U/L)] | 134 | 69 | 105 | 84 | 54-77 |
| [GGT (U/L)] | 0 | 0 | 0 | 0 | — |
| [TBIL (mg/dL)] | 0.1 | 0.1 | 0.1 | 0.2 | 0.2-0.6 |
| [DBIL (mg/dL)] | 0.0 | 0.0 | 0.0 | 0.0 | — |
| IBIL | 0.1 | 0.1 | 0.1 | 0.2 | — |
| [TP (g/dL)] | 5.4 | 4.5 | 5.2 | 5.3 | 4.8-7.2 |
| [ALB (g/dL)] | 3.1 | 2.6 | 3.2 | 3.2 | 2.4-4.3 |
| GLOB (g/dL) | 2.3 | 1.9 | 2 | 2.1 | 1.7-2.2 |
| A/G Ratio | 1.3 | 1.4 | 1.6 | 1.5 | — |
| [P (mg/dL)] | 10.8 | 10.1 | 9.9 | 11.4 | 7.3-14.5 |
| [Ca (mg/dL)] | 10.0 | 9.9 | 9.7 | 10.0 | 9.5-12.5 |
| [GLU (mg/dL)] | 205 | 210 | 220 | 212 | 172-372 |
| [CHOL (mg/dL)] | 103 | 93 | 90 | 109 | 55-169 |
| [TRIG (mg/dL)] | 82 | 80 | 124 | 100 | 67-289 |
| [CK (U/L)] | 540 | 109 | 290 | 82 | 428-1609 |
| [TCO2 (mEq/L)] | 24 | 18 | 27 | 24 | — |
| [Na (mEq/L)] | 157 | 153 | 154 | 160 | 145-181 |
| [K (mEq/L)] | 8.1 | 9.7 | 7.4 | 7.1 | 7.3-11.1 |
| [CL (mEq/L)] | 115 | 113 | 110 | 116 | 111-134 |
| [Na/K] | 19 | 16 | 21 | 23 | — |
| [Anion Gap] | 26 | 32 | 24 | 27 | — |
| BASELINE | 0.811 | 0.662 | 1.21 | 1.71 | — |
| CORTISOL | | | | | |

TABLE 10
Blood counts of mice treated with C-176 and vehicle control

| | C-176 | | Vehicle | | Reference |
|---|---|---|---|---|---|
| Animal | 1 | 2 | 1 | 2 | Range |

COMPLETE BLOOD COUNT

| | | | | | |
|---|---|---|---|---|---|
| RBC (M/uL) | 10.18 | 9.5 | 9.9 | 10.35 | 6.93-12.24 |
| HGB (g/dL) | 15.3 | 14.4 | 15.3 | 15.9 | 12.4-20.5 |
| HCT (%) | 47.3 | 45.2 | 48.9 | 50.1 | 42.1-68.3 |
| MCV (fL) | 46.5 | 47.6 | 49.4 | 48.4 | 50.7-64.4 |
| MCH (pg) | 15 | 15.2 | 15.5 | 15.4 | 13.0-17.6 |
| MCHC (g/dL) | 32.3 | 31.9 | 31.3 | 31.7 | 23.3-33.1 |
| RDW-SD (fL) | 29.1 | 29.6 | 29.8 | 30.1 | — |
| RDW-CV (%) | 25.4 | 24 | 23.7 | 25 | 16.9-23.5 |
| RET# (K/uL) | 411.3 | 502.6 | 522.7 | 373.6 | 294-444 |
| RET (%) | 4.04 | 5.29 | 5.28 | 3.61 | 2.56-4.56 |
| PLT (K/uL) | 1137 | 1026 | 836 | 937 | 420-1698 |
| PDW (fL) | 9.9 | 10.3 | 9.9 | 9.3 | — |
| MPV (fL) | 7 | 7.3 | 7 | 8.5 | 4.6-5.9 |

AUTOMATED DIFFERENTIAL

| | | | | | |
|---|---|---|---|---|---|
| WBC# (K/uL) | 6.65 | 8.25 | 4.87 | 3.70 | 3.48-14.84 |
| NEUT# (K/uL) | 2.98 | 4.69 | 0.93 | 0.93 | 0.58-3.83 |
| LYMPH# (K/uL) | 3.35 | 3.14 | 3.58 | 2.45 | 2.22-11.56 |
| MONO# (K/uL) | 0.22 | 0.25 | 0.2 | 0.22 | 0.21-1.37 |
| EO# (K/uL) | 0.1 | 0.16 | 0.15 | 0.10 | 0.01-0.49 |
| BASO# (K/uL) | 0 | 0.01 | 0.01 | 0.00 | 0.00-0.18 |
| NEUT (%) | 44.8 | 56.9 | 19.1 | 25.2 | 9.86-39.11 |
| LYMPH (%) | 50.4 | 38.1 | 73.5 | 66.2 | 48.81-83.82 |
| MONO (%) | 3.3 | 3 | 4.1 | 5.9 | 3.29-14.33 |
| EO (%) | 1.5 | 1.9 | 3.1 | 2.7 | 7 0.07-4.91 |
| BASO (%) | 0 | 0.1 | 0.2 | 0.0 | 0.00-1.84 |

MANUAL DIFFERENTIAL

| | | | | | |
|---|---|---|---|---|---|
| NEUT (%) | 45 | 57 | 16 | 31 | 9.86-39.11 |
| BAND (%) | 1 | — | — | — | — |
| LYMPH (%) | 52 | 42 | 82 | 67 | 48.81-83.82 |
| MONO (%) | — | — | — | — | 3.29-14.33 |
| EO (%) | 2 | 1 | 2 | — | 0.07-4.91 |
| BASO % | — | — | — | — | 0.00-1.84 |
| Other 1% | — | — | — | 2 | — |

### Example 5: Chronic cGAS-STING Signaling is Associated with Poor Prognosis in Human Cancer

Building on this experimental insight, the dynamics of cGAS and STING in human cancers was investigated. Three commercially available anti-human cGAS antibodies were validated using paraformaldehyde-fixed cell pellets from control or cGAS-depleted MDA-MB-231 human TNBC cells. Two out of the three antibodies displayed diffuse cytoplasmic staining in both control and cGAS-depleted cell pellets, suggesting non-specific binding (FIGS. 10A-10B). A third antibody (LS-C757990) revealed specific staining in micronuclei, which was lost upon shRNA-mediated cGAS depletion (FIGS. 10A-10B).

With this validated antibody, cGAS in 24 human mucosal melanoma samples, for which targeted exome and low-pass whole genome sequencing was available through the MSK-IMPACT panel, and in a cohort of 179 TNBC tumors for which long-term clinical follow-up was available, were stained. cGAS was primarily localized to micronuclei and this conspicuous punctate staining pattern was seen in 100% and 92% of human mucosal melanoma and TNBC samples, respectively (FIGS. 5A and 10C)—arguing against widespread loss of cGAS protein expression across human cancers. Interestingly, the frequency of micronuclear cGAS staining was highly correlated with genomic copy number alterations (R²=0.54, p<0.0001)—but not with the tumor mutational burden (R²=0.004, p=0.76) (FIG. 5B), suggesting that this staining pattern may serve as a surrogate marker for CIN in human cancer.

In line with our findings in cancer cells, an inverse correlation between the frequency of micronuclei with cGAS staining and tumor cell-intrinsic STING expression in human TNBC was observed (FIG. 5C). Tumors with a preponderance of micronuclei with cGAS staining had low, but detectable, STING protein levels within cancer cells, in line with increased protein turnover. This observation held true even within individual tumor samples in which cGAS and STING protein expression exhibited spatial heterogeneity (FIG. 5D). Increased frequency of micronuclei with cGAS localization was associated with reduced distant metastasis-free survival (DMFS), whereas the opposite was observed with cancer cell-intrinsic STING levels (FIG. 10D). Furthermore, patients with cGAS^{high}STING^{low} tumors had the highest odds for distant metastasis whereas with those with cGAS^{low}STING^{high} tumors had the most favorable prognosis (FIG. 5E). Unlike cancer cells, stromal cells consistently displayed strong STING protein expression levels, exhibiting little evidence for cGAS staining in micronuclei. Based on experimental and clinical data, it is proposed that the widespread prevalence of micronuclei with cGAS staining, coupled with reduced cancer cell-intrinsic STING levels, represent a subset of aggressive chromosomally unstable cancers wherein the cytosolic dsDNA-sensing pathway is chronically stimulated.

Collectively, these results demonstrate that: STING inhibitors are useful in methods for treating cancer associated with increased cGAS⁺ micronuclei and decreased STING protein expression; and cGAS inhibitors are useful in methods for treating cancer associated with increased cGAS⁺ micronuclei and decreased STING protein expression when administered prior to the administration of a STING inhibitor, as compared to that observed in a reference sample.

### Example 6: cGAMP is Readily Exported from Cancer Cells

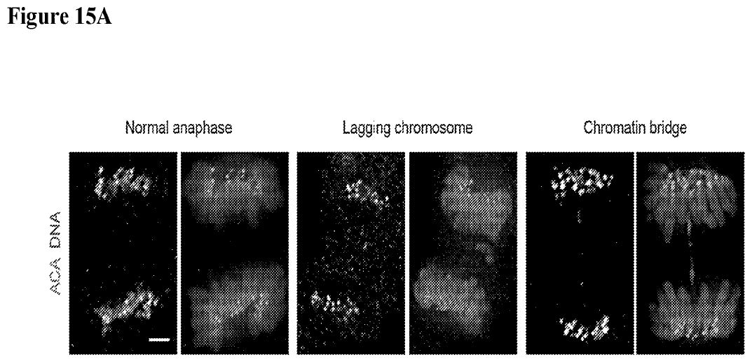

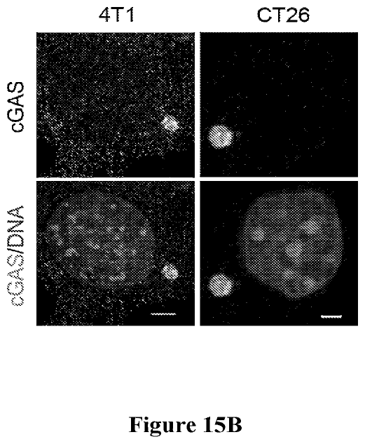

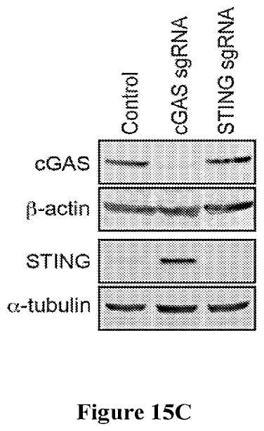

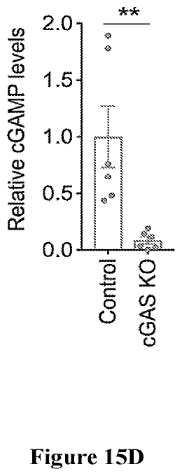

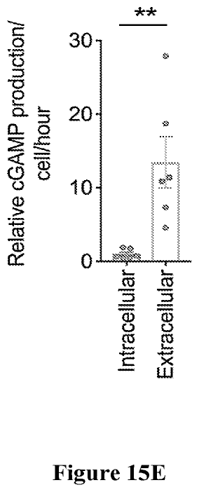

In this experiment, two syngeneic mouse models of triple-negative breast cancer (TNBC) metastasis (4T1) and colorectal cancer metastasis (CT26) were used. As expected, both models exhibited high levels of CIN, as evidenced by the presence of chromosome missegregation during anaphase and a preponderance of micronuclei with robust cGAS localization (FIGS. 15A-15B). cGAMP was readily detectable in total 4T1 cell lysates and CRISPR-Cas9 mediated knockout of Cgas resulted in a significant reduction in the levels of this metabolite (FIGS. 15C-15D). Furthermore, cGAMP levels were nearly 15-fold higher in conditioned media after 24 hr, as compared to cell lysates, when both were normalized to cell counts (FIG. 15E), suggesting that cGAMP is readily exported from cancer cells.

### Example 7: ENPP1 Expression is Elevated in Highly Metastatic (CIN^{high}) Tumor Cells

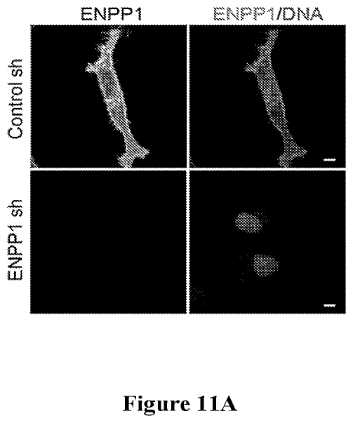

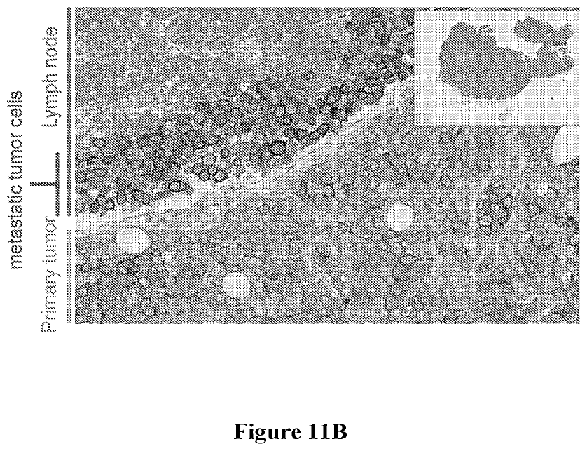

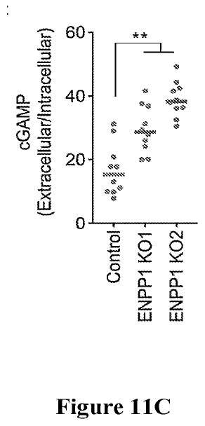

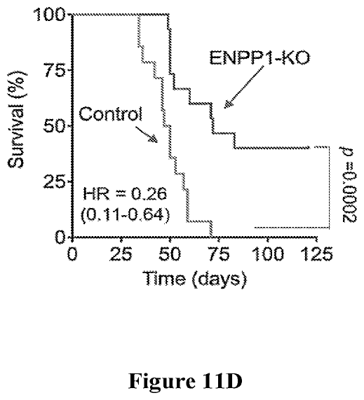

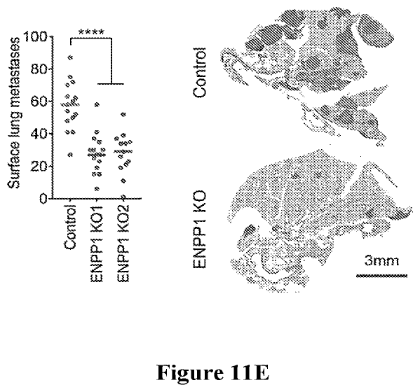

To determine how chromosomally unstable cells cope with ongoing cGAMP production, otherwise isogenic human MDA-MB-231 TNBC cells that were engineered to exhibit different rates of CIN through overexpression of the kinesin-13 proteins, Kif2b or MCAK, or the dominant-negative mutant isoform of MCAK (dnMCAK) were used. Pairwise differential expression analysis comparing CIN^{high} (highly metastatic) to CIN^{low} (poorly metastatic) cells revealed a large number of differentially expressed genes, among which ENPP1 stood out (FIG. 16A, Log 2 fold change=1.23, FDRq=8.4×10⁻⁴) because of its role as an ectonucleotidase that hydrolyses cGAMP in the extracellular space. Protein levels of ENPP1 were markedly increased in CIN^{high} cells compared with their CIN^{low} counterparts and staining using an anti-ENPP1 antibody revealed strong membrane localization that was abolished upon shRNA-mediated depletion (FIG. 11A and FIGS. 16B-16D). Similar to findings in human metastatic MDA-MB-231 cells, there was a significant increase in ENPP1 messenger RNA (mRNA) levels in mouse lung metastasis-derived 4T1 cells as compared with the parental cell line (FIG. 16E). And, even within orthotopically transplanted tumors, increased expression of ENPP1 was observed selectively in tumor cells invading nearby intra-mammary lymph nodes (FIG. 11B), suggesting that ENPP1 might play a role in promoting metastasis in chromosomally unstable cells.

### Example 8: ENPP1 Loss Results in Increased Survival, Reduced Local Tumor Recurrence, and Reduced Metastasis

To evaluate the role of ENPP1 in metastasis in an immune-competent setting, CRISPR-Cas9 knockout (KO) of Enpp1 in 4T1 cells was performed (FIG. 16F). As expected, loss of ENPP1 led to a significant increase in the extracellular-to-intracellular cGAMP ratio (FIG. 11C). Then parental and Enpp1-KO 4T1 cells were transplanted into BALB/c hosts either through tail vein inoculation or orthotopic transplantation into the mammary fat pad followed by primary tumor excision. Enpp1-KO did not impact cellular proliferation in vitro or primary tumor growth in vivo when cells were orthotopically transplanted in the mammary fat pad (FIGS. 16G-16H). However, loss of ENPP1 led to significantly longer overall survival and a marked reduction in local tumor recurrence and metastasis regardless of whether cells were introduced directly into the tail vein or orthotopically transplanted followed by surgical excision of the primary tumor (FIGS. 11D-11E and FIGS. 16I-16L). These results suggest that disrupting the ability of chromosomally unstable cancer cells to sequester cGAS-STING signaling, by increasing extracellular cGAMP availability, might suppress metastatic progression.

### Example 9: Adenosine is a Breakdown Product of cGAMP

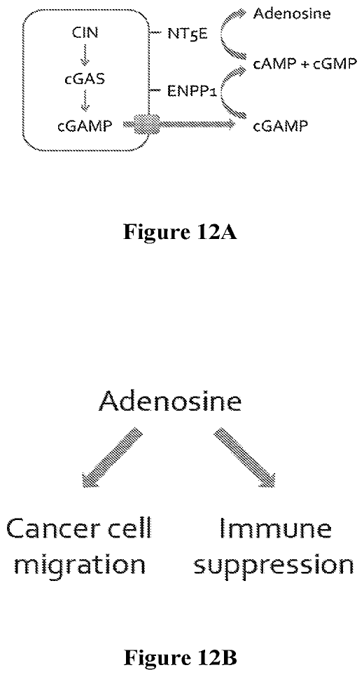

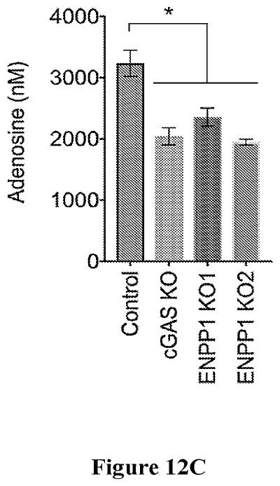

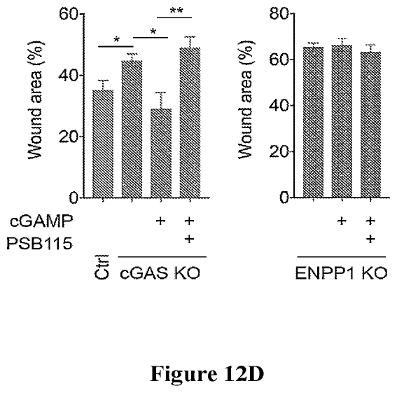

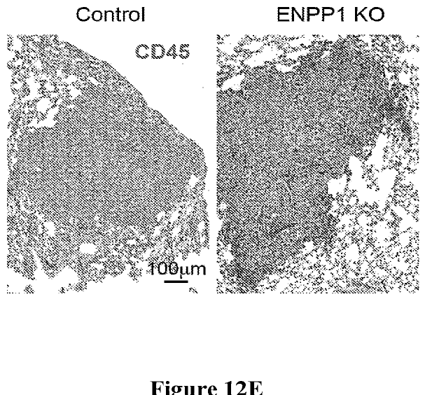

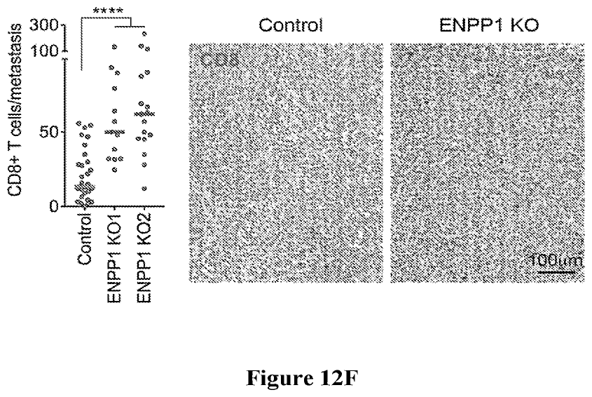

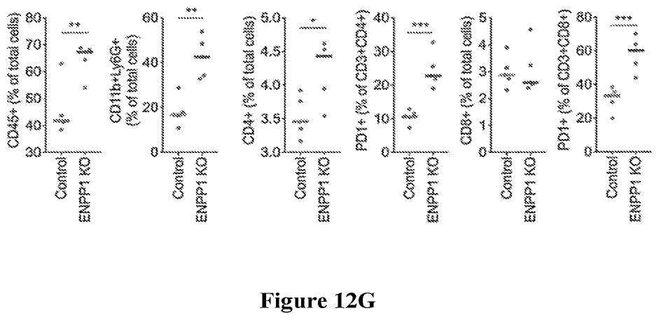

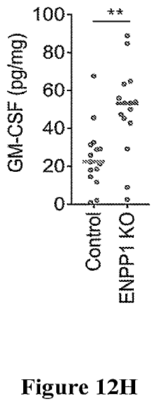

To further explore the fate of tumor-derived extracellular cGAMP, whether the breakdown products of this metabolite might contribute to the production of adenosine, an immune-suppressive and tumor-promoting molecule, was investigated (FIGS. 12A-12B). Measuring adenosine in conditioned media is technically challenging given the presence of enzymes that either degrade this nucleoside (adenosine deaminase, ADA) or promote its cellular reuptake (FIG. 17A). To overcome these challenges, serum-free media was added to 4T1 cells in the presence of erythro-9-(2-hydroxy-3-nonyl)adenine (EHNA), an ADA inhibitor, along with dipyridamole and 6-S-[(4-Nitrophenyl)methyl]-6-thioinosine (NBMPR), which prevent cellular re-uptake of adenosine (FIG. 17A). Extracellular adenosine levels—as assessed by liquid chromatography-mass spectrometry in conditioned media—were reduced by up to 40% upon knockout of either Cgas or Enpp1 (FIG. 12C). Using an orthogonal approach, exogenous cGAMP was added to 4T1 cells and a fluorescence-based method was used to detect hydrogen peroxide (H₂O₂) resulting from the oxidation of hypoxanthine, a breakdown product of adenosine (FIG. 17A). By comparing fluorescence in the presence and absence of EHNA, relative contribution from adenosine degradation toward H₂O₂ production was assessed and a concentration-dependent increase in H₂O₂ production after the addition of exogenous addition of cGAMP was observed (FIG. 17B), suggesting that this cyclic dinucleotide can be readily converted into adenosine in the extracellular environment.

Through its ability to bind extracellular adenosine receptors in both tumor and immune cells, adenosine promotes cancer cell migration and is a potent immune suppressor, respectively (FIG. 12B). Interestingly, knockout of either Cgas or Enpp1 led to a significant reduction in cancer cell migration, whereas exogenous addition of cGAMP rescued migration only in Cgas-KO but not Enpp1-KO tumor cells (FIG. 12D). The effect of cGAMP was dependent on adenosine receptor activity and was abolished upon the addition of PSB115, an inhibitor of the adenosine A2B receptor on cancer cells (FIG. 12D).

### Example 10: ENPP1 Loss Results in Increased Tumor Immune Infiltration

Next, the effect of ENPP1 loss on tumor immune infiltration using shRNA-mediated depletion or CRISPR-Cas9-mediated knockout in CIN^{high} MDA-MB-231 orthotopic xenografts and 4T1 allografts, respectively, was examined. Loss of ENPP1 led to increased tumor necrosis and enhanced infiltration of natural killer (NK)-cells in MDA-MB-231 tumors (FIGS. 18A-18B). In the 4T1 model, Enpp1-KO metastatic lesions exhibited significant infiltration by CD45+ cells and a ˜3-5-fold enrichment with CD8+ T-cells compared to wildtype counterparts (FIGS. 12E-12F). Flow cytometry-based immune profiling of dissociated lungs revealed a significant increase in CD45+ cells, CD4+ T-cells as well as granulocytic CD11b+Ly6G+ cells as compared to controls (FIG. 12G and FIG. 18C). There was no overall enrichment for CD8+ T-cells in the injected lungs however there was a significant increase in PD1+ subpopulations of CD3+CD8+ and CD3+CD4+ cells (FIG. 12G). The overall preponderance of granulocytic cells was notable, given that ENPP1-KO tumors had higher levels of GM-CSF as measured using ELISA-based assays (FIGS. 12G-12H). These findings suggest that granulocytic cells may play a role in restricting metastatic colonization of Enpp1-KO cells in line with previous reports showing an anti-tumor and pro-inflammatory effect of CD11b+Ly6G+ cells.

### Example 11: ENPP1 Depletion Sensitizes Otherwise Resistant Chromosomally Unstable Tumors to Immune Checkpoint Blockade Therapy

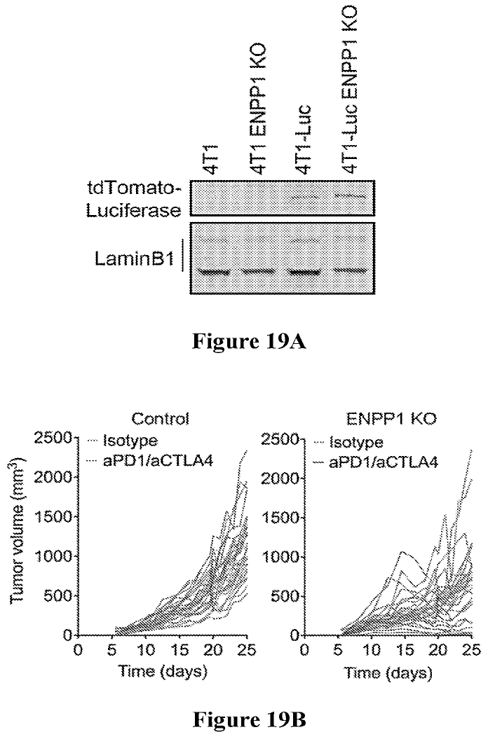

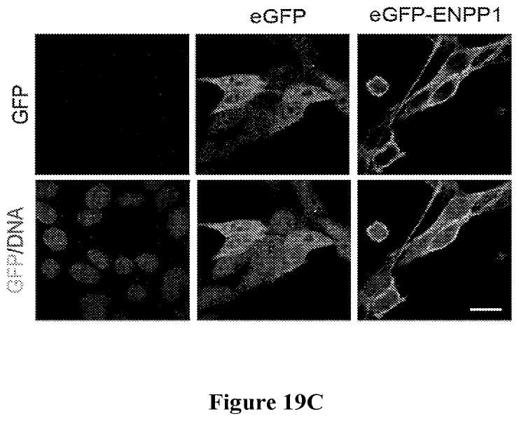

In this example, whether ENPP1 depletion might be used as a therapeutic vulnerability to sensitize otherwise resistant chromosomally unstable tumors to immune checkpoint blockade (ICB) was investigated. Interestingly, ENPP1 mRNA expression was significantly higher in 4T1 cells compared to CT26 (FIG. 13A). It was postulated that Enpp1 knockout would render 4T1 tumors responsive to ICB whereas its overexpression would confer resistance to CT26 tumors. Luciferase-expressing 4T1 cells were orthotopically transplanted into the mammary fat pad of BALB/c mice and primary tumor growth were assessed over the span of 25 days (FIG. 13B and FIGS. 19A-19B). Animals were treated with combined ICB (anti-PD1 and anti-CTLA4) starting at day 6 after tumor cell inoculation for 4 doses followed by maintenance aCTLA4 treatment every 3 days. Enpp1-KO tumors exhibited a profound response to combined ICB when compared to wildtype counterparts, leading to significantly prolonged survival (FIGS. 13C-13D). To test if overexpression of ENPP1 would confer treatment resistance in otherwise sensitive CT26 tumors, eGFP or eGFP-ENPP1 was expressed in tail vein-injected CT26 cells (FIG. 13B and FIG. 19C). Mice were treated with combined ICB starting at day 6 for a total of 5 doses. Strikingly, not only did eGFP-ENPP1 expression lead to increased metastasis and reduced survival of isotype control-treated mice, it also rendered this model completely resistant to combined ICB (FIGS. 13E-13F). Conversely, eGFP-expressing CT26 tumors were responsive to combined ICB with 60% of animals surviving for over 90 days, supporting a role for ENPP1 in metastasis and immune evasion of chromosomally unstable tumors.

### Example 12: High ENPP1 Expression is Associated with Reduced Survival, Increased Metastasis, and Disease Relapse in Human Cancers

Next, the role of ENPP1 in human cancers was investigated. ENPP1 mRNA expression across the TCGA, two independent sarcoma cohorts, and in tumor-derived organoids, was analyzed. ENPP1 protein expression in three independent breast cancer cohorts, including two estrogen-receptor-negative (ER-) cohorts (n=223 and 91) and one estrogen receptor-positive (ER+) cohort (n=115), was probed. Finally, ENPP1 expression in mucosal melanoma primary and metastatic tumors (n=24) was evaluated. Unlike cutaneous melanoma, mucosal melanoma is characterized by elevated CIN, reduced tumor mutational burden, and increased resistance to immune checkpoint blockade.

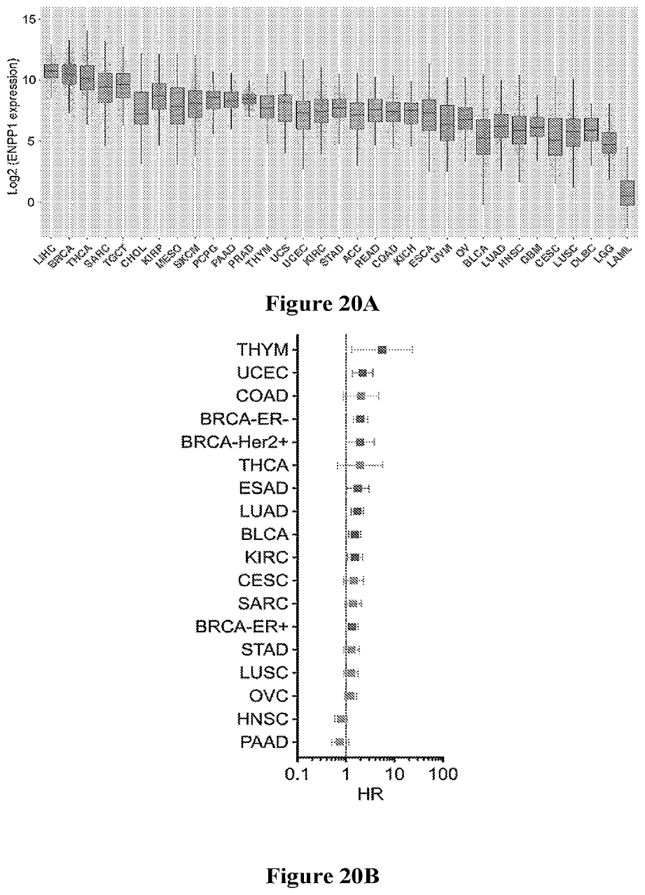

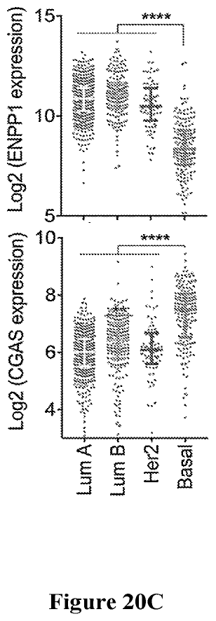

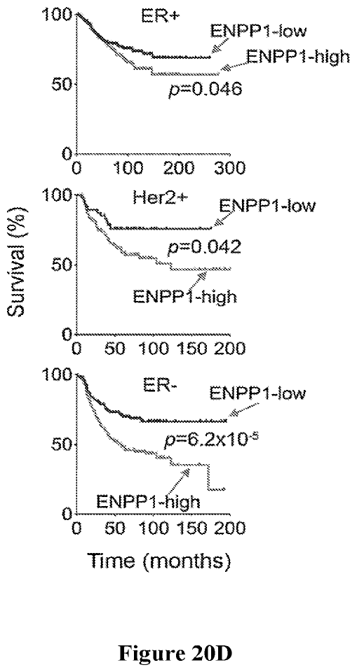

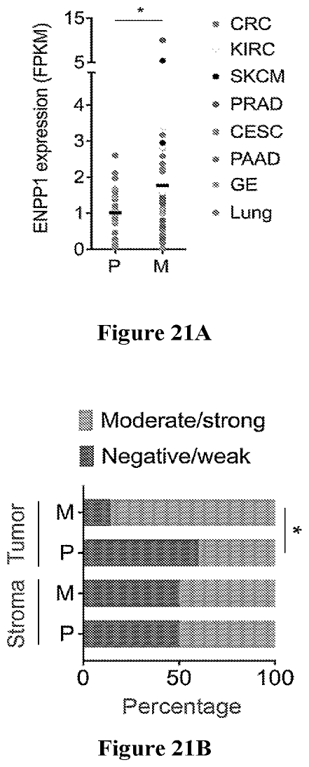

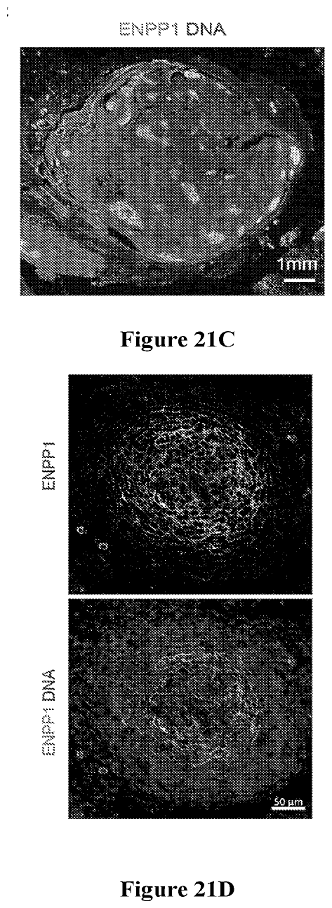

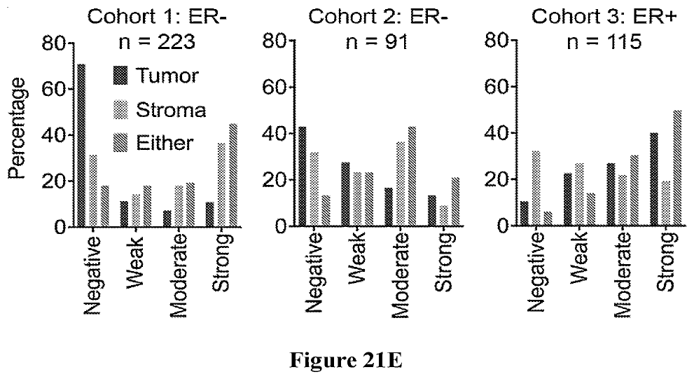

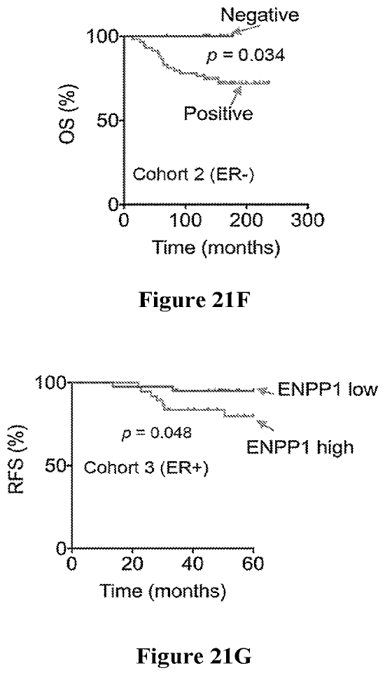

ENPP1 mRNA expression was highly variable across cancer types, with highest expression levels observed in sarcomas, liver, breast, and thyroid cancers (FIG. 20A). Elevated ENPP1 mRNA was associated with reduced overall survival in multiple tumor types including breast cancer, irrespective of the hormone receptor status (FIGS. 20B-20D). In line with these findings, ENPP1 mRNA levels were found to be significantly higher in organoids derived from human metastatic tumors compared to those originating from primary tumors (FIG. 21A). This was also validated in mucosal melanomas, where metastases displayed increased cancer cell-specific ENPP1 staining intensity (FIG. 21B). Tumor cell-intrinsic ENPP1 protein expression was most remarkable in lymph-node metastases where cancer cell clusters exhibited strong ENPP1 expression in an otherwise immune-cell replete microenvironment (FIGS. 21C-21D). In primary breast tumors, three distinct patterns of ENPP1 protein expression: tumor-cell-dominant, stroma-dominant, and negative, were observed (FIG. 14A and FIG. 21E). Overall, 64% of primary TNBCs exhibited moderate or strong ENPP1 staining in either tumor cells or the stroma—a distribution that was consistent across the two ER-cohorts—compared with 90% of ER+ tumors, which displayed enrichment for tumor-cell intrinsic expression (FIG. 21E). Across all three cohorts, high ENPP1 protein expression was associated with reduced survival and increased metastasis and disease relapse (FIG. 14B and FIGS. 21F-21G).

Accordingly, these results demonstrate that ENPP1 inhibitors are useful in methods for treating cancer associated with increased ENPP1 protein expression levels and/or increased cGAS⁺ micronuclei and increased ENPP1 protein expression as compared to that observed in a reference sample.

### Example 13: ENPP1 Expression Levels Inversely Correlate to Tumor Lymphocyte Infiltration

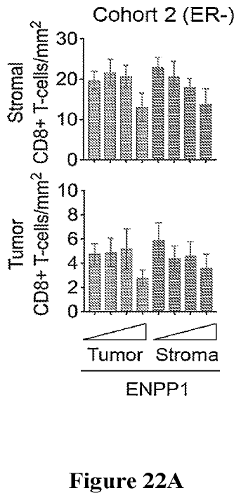

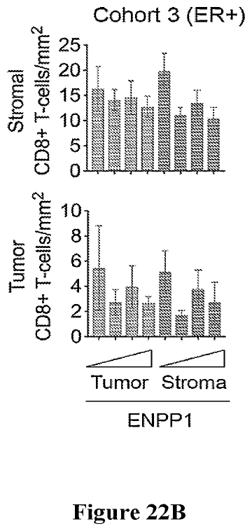

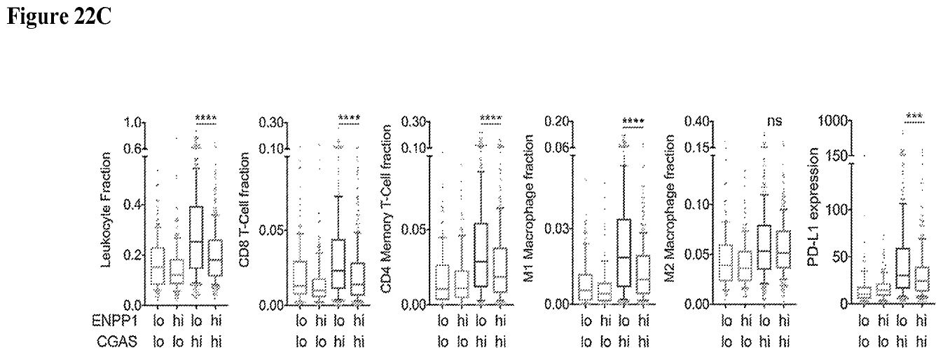

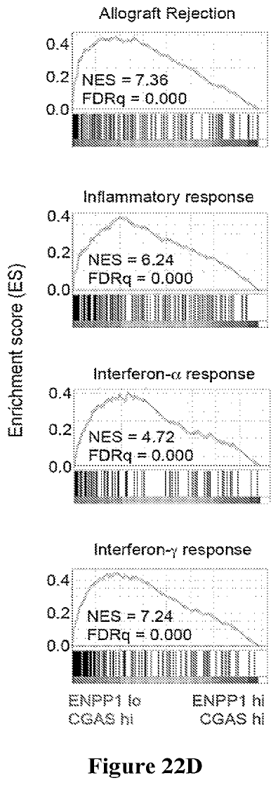

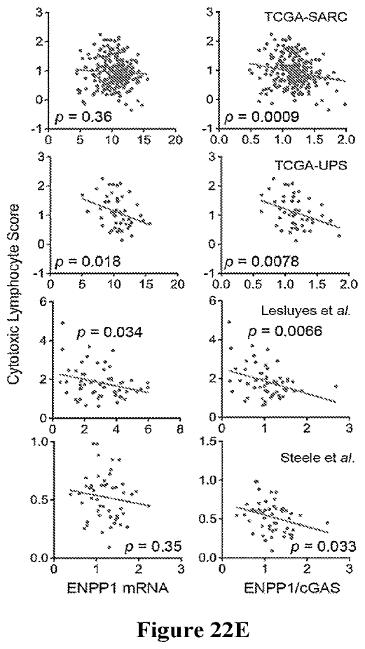

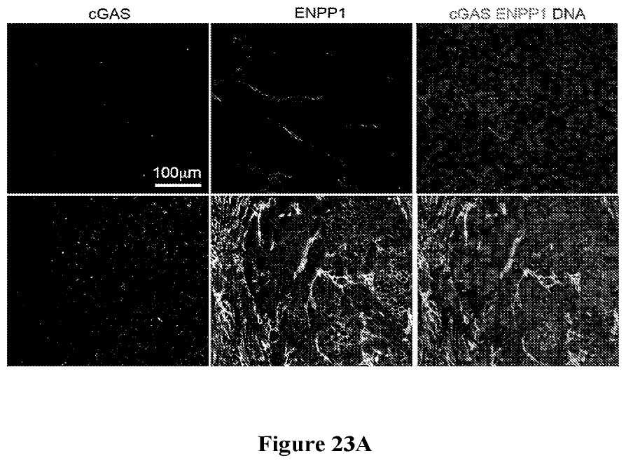

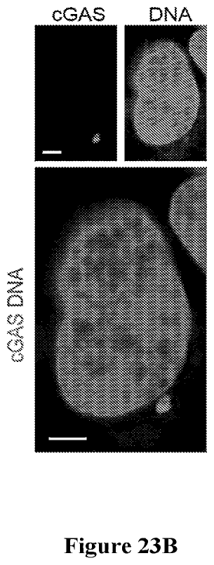

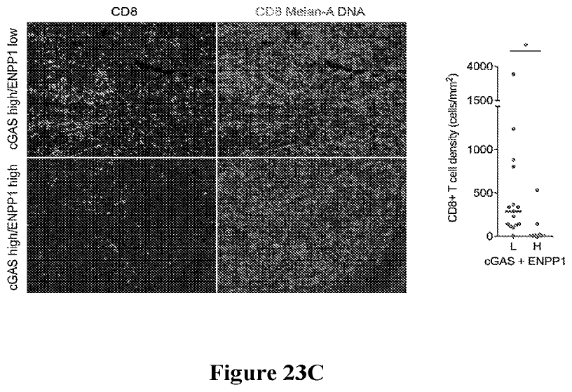

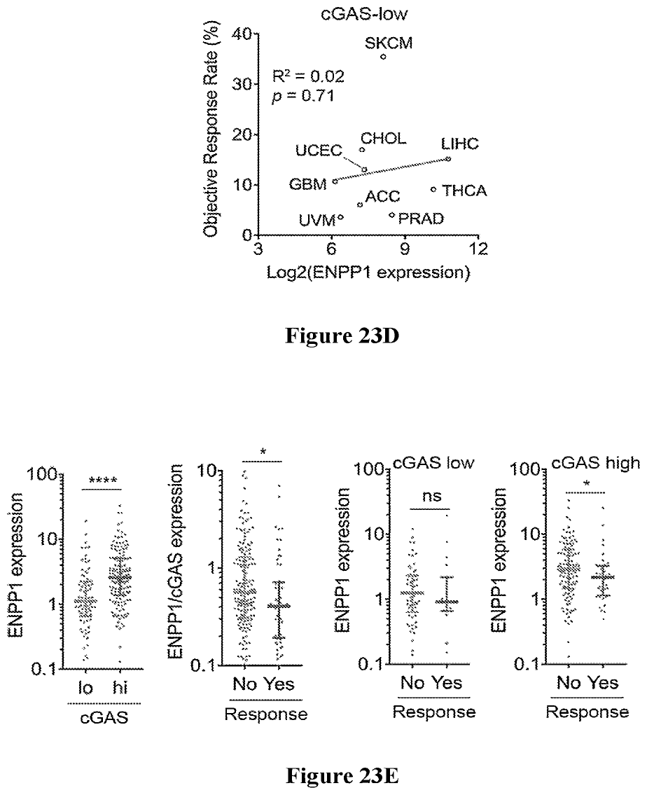

Next, ENPP1 protein levels with tumor-infiltrating lymphocytes (TILs) and CD8+ T-cell density across breast cancers were correlated, and an inverse correlation between ENPP1 IHC expression intensity and lymphocytic infiltration was found (FIGS. 14C-14D and FIG. 22A-22B). It was reasoned that if ENPP1 impacts tumor immune infiltration through cGAMP degradation, then its levels would be solely relevant in tumors with high levels of cGAS. The 1,079 breast cancers found in the TCGA were segregated into four subsets based on their relative CGAS and ENPP1 expression levels and used the CIBERSORT method to infer the prevalence of immune cell subsets from tissue expression profiles. Expectedly, ENPP1 expression was minimally associated with the immune cell fraction in tumors with low CGAS expression, whereas in those with high CGAS mRNA, it was inversely correlated with the overall leukocyte fraction as well as with the proportion of CD8+ T-cells, CD4+ T-cells, and pro-inflammatory macrophages in tumors with elevated CGAS mRNA (FIG. 22C). Interestingly, PD-L1 expression had a similar pattern, with the highest levels seen in tumors with high CGAS and low ENPP1 expression. Gene Set Enrichment Analysis (GSEA) comparing cGAS^{high}ENPP1^{high} to cGAS^{high}ENPP1^{low} breast tumors revealed upregulation of inflammatory pathways related to allograft rejection, type I interferon, and interferon-γ-associated responses in the latter subset of tumors (FIG. 22D). These findings suggest that ENPP1-to-cGAS ratio might be more predictive of tumor immune infiltration compared to ENPP1 expression levels alone. This assumption was orthogonally validated in sarcomas and mucosal melanoma tumors. In sarcomas, ENPP1-to-CGAS expression ratio was more strongly associated with the cytotoxic lymphocyte score compared with ENPP1 expression levels alone (FIG. 22E). In mucosal melanomas, tumors with numerous cGAS-positive micronuclei and low ENPP1 expression exhibited increased CD8+ T-cell density, whereas those with elevated ENPP1 expression in the setting of widespread cGAS-positive micronuclei exhibited significantly reduced CD8+ T-cell infiltration (FIGS. 23A-23C).

### Example 14: Low ENPP1:cGAS Ratios Correlate to Tumor Response to Immune Checkpoint Blockade Therapy

In line with its role modulating tumor immune responses, it was found that ENPP1 expression within a given cancer type negatively correlates with its overall response rate to anti-PD1/PD-L1 therapy. This inverse association was again restricted to tumor types characterized by elevated levels of cGAS expression (FIG. 14E and FIG. 23D). Next, the mRNA expression levels of cGAS and ENPP1 in 228 bladder cancers treated with anti-PD-L1 (aPD-L1) therapy were analyzed. There was an overall positive correlation between CGAS and ENPP1 expression and ENPP1 levels were significantly lower in CGASgh tumors that responded to a PD-L1 therapy. A low ENPP1-to-cGAS expression ratio was significantly correlated to tumor response across the entire cohort (FIG. 23E).

Accordingly, these results demonstrate that immune checkpoint blockade agents are useful in methods for treating cancer associated with a low ENPP1 to cGAS expression ratio.

### Example 15: Therapeutic Synergy Between Combined ENPP1 and NT5E Loss of Function

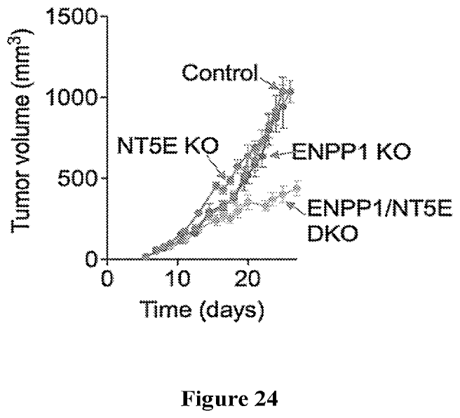

Tumor volume over time of orthotopically transplanted wildtype, ENPP1 knockout, NT5E knockout, or ENPP1/NT5E double knockout 4T1 triple negative breast tumors shows reduced tumor growth in an ENPP1 and NT5E double knockout (FIG. 24). These results suggest that dual inhibition of NT5E (also known as CD73) and ENPP1 can be therapeutically beneficial in the treatment of cancer.

NT5E inhibitors have been developed and have had limited efficacy in the clinic. However, as the results shown in FIG. 24 demonstrate, co-inhibition of ENPP1 and NT5E may provide the clue to unlocking this therapeutic class. Without wishing to be bound by theory, the synergy in the reduction of tumor volume may be due to complete inhibition of the extracellular adenosine pathway while simultaneously increasing extracellular cGAMP levels in the tumor microenvironment leading to enhanced anti-tumor immune responses. These two enzymes are extracellular, and, as such, can be targeted with a small molecule inhibitor, an inhibitory antibody, or a LYTAC. This dual blockade is expected to further synergize with immunotherapy, chemotherapy, and radiation therapy treatments.

Accordingly, these results demonstrate that the combination of ENPP1 inhibition and NT5E inhibition is useful in methods for treating cancer.

### Example 16: Therapeutic Synergy Between Activation of STING and Inhibition of NT5E

To test the effect of pharmacologic STING activation and inhibition of NT5E on cancer metastasis, a STING agonist and an NT5E inhibitor are delivered through daily IP injections to tumor bearing animals and both survival and the development of metastases are monitored.

It is anticipated that these results will demonstrate that activation of STING in combination with NT5E inhibition are therapeutically beneficial in the treatment of cancer. It is also expected that activation of STING and inhibition of NT5E will further synergize with immunotherapy, chemotherapy, and radiation therapy treatments.

Accordingly, these results will demonstrate that the combination of STING activation and NT5E inhibition is useful in methods for treating cancer.

### Example 17: Using Micronuclei as a Surrogate Measure of Chromosomal Instability

This example demonstrates a method for measuring the presence of cGA⁺ micronuclei in a tumor sample as a surrogate for detecting chromosomal instability in the tumor sample. This example also highlights some of the advantages of the claimed technology, including the importance of measuring CIN directly rather than relying on, e.g., NexGen techniques that infer aneuploidy.

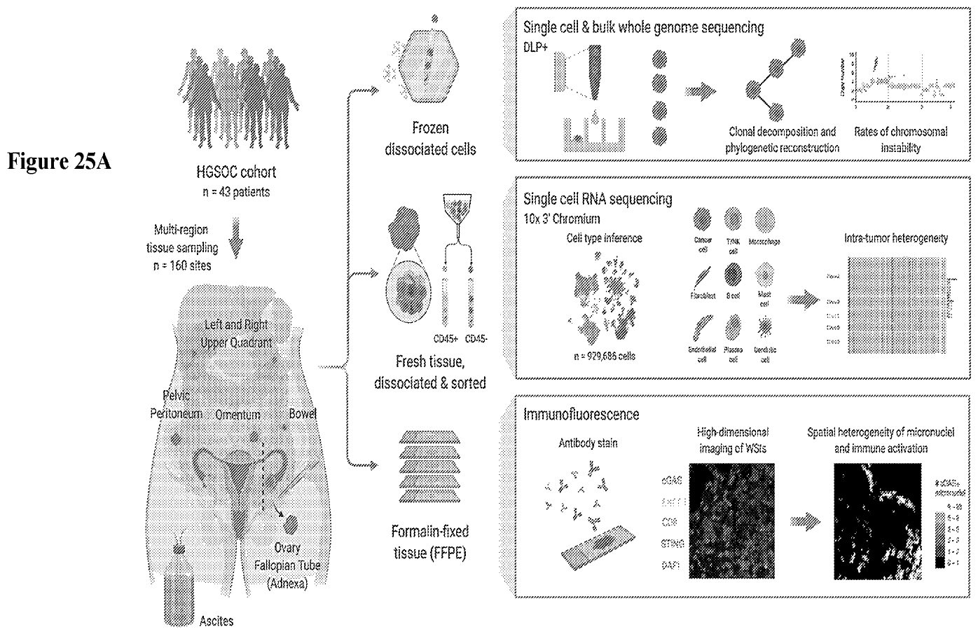

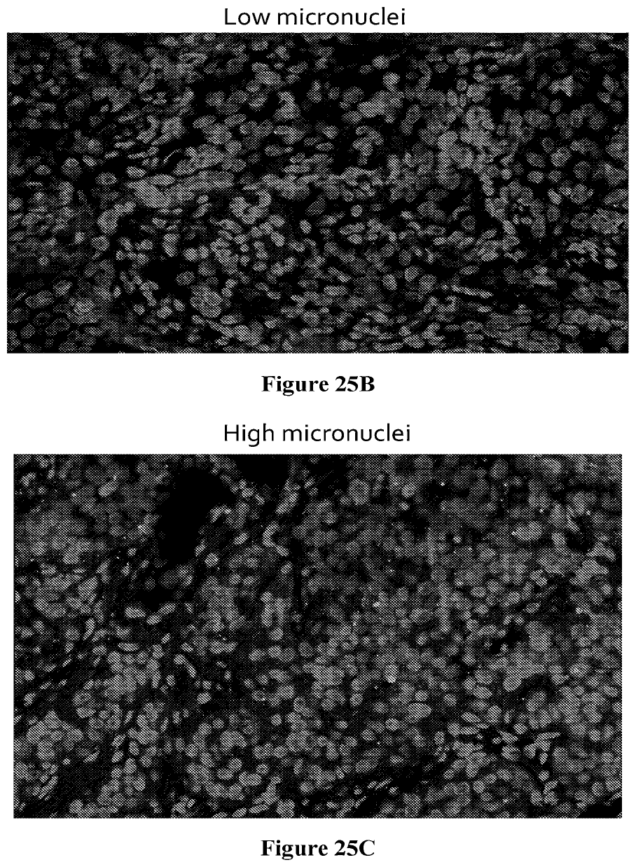

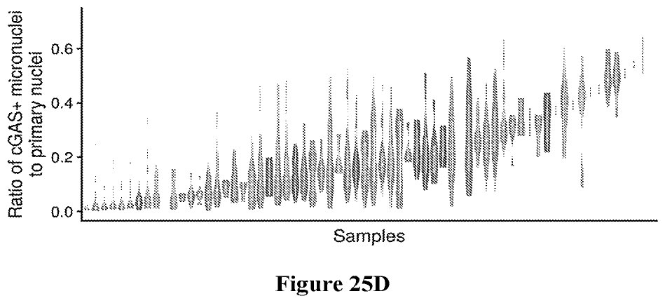

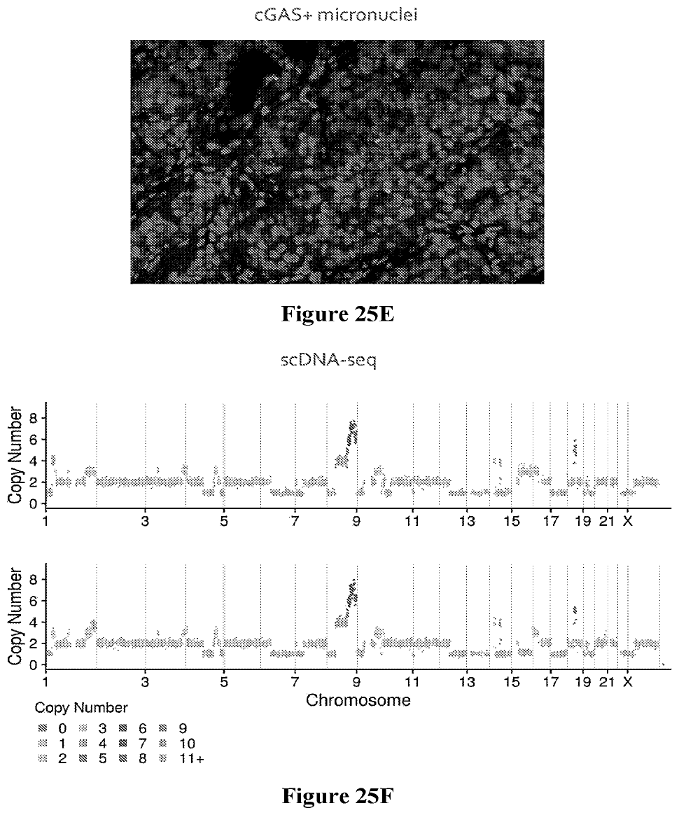

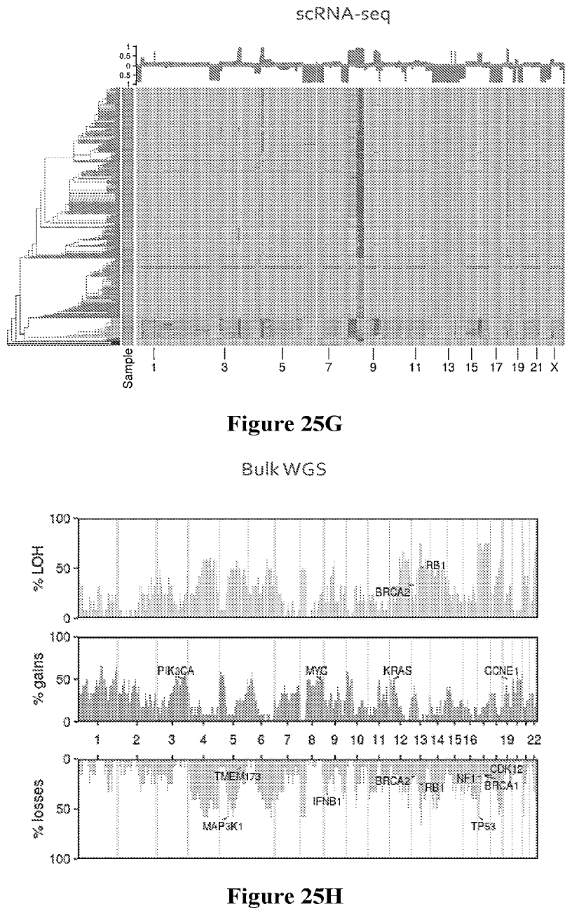

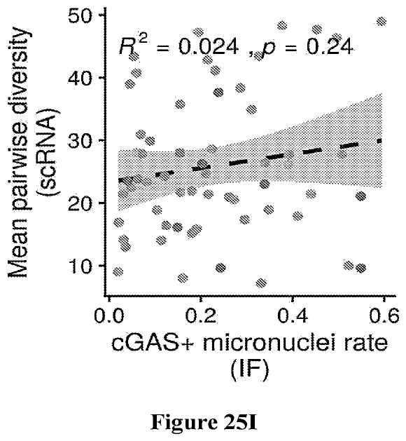

Methods. Briefly, 100 human high-grade serous ovarian cancer (HGSOC) samples from a cohort of 43 patients from MSK were analyzed for the presence of cGAS⁺ micronuclei along with other metrics such as single cell DNA sequencing, single cell RNA sequencing, and bulk whole genome sequencing to validate the use of cGAS⁺ micronuclei as a surrogate marker for chromosomal instability and to analyze its relationship to various outcomes (FIG. 25A). cGAS staining was carried out as described in Li et al. 2021 Cancer Discovery PMID: 33372007. scRNAseq data was done using the commercial 10× platform. The single cell DNA sequencing was done as described in Laks et al. Cell 2019, PMID: 31730858.

Results. The method described above was applied in a scalable manner to 100 tumor samples at minimal cost and labor as compared to other more laborious or expensive methods that rely on NexGen sequencing (expensive) or karyotyping (laborious and low throughput). Illustrative “low micronuclei” and “high micronuclei” samples are shown in FIGS. 25B and 25C, respectively. As shown in FIG. 25D, a large degree of heterogeneity in patient samples (even within individual patients) was observed spanning low levels of chromosomal instability (or cGAS⁺ micronuclei), from almost 0% to over 60%, suggesting a good dynamic range.

Analysis of cGAS⁺ micronuclei as a metric for CIN revealed that this metric does not correlate with copy number alterations/aneuploidy (as measured by single cell RNAseq and bulk whole genome sequencing). The single cell RNAseq (scRNA-seq) is called mean pairwise distance (MPD). The frequency of cGAS⁺ micronuclei (as measured by cGAS staining and defined as the number of micronuclei divided by the total number of primary nuclei; FIG. 25E provides an example of a high-grade serous ovarian cancer sample stained with DAPI for DNA and anti-cGAS antibody), however, correlated with missegregation rates inferred from single cell DNA sequence data (scDNA-seq) (FIG. 25F). Illustrative aneuploidy/heterogeneity results are shown in FIGS. 25G and 25H. Collectively, this data: (1) highlights the relevance and utility of cGAS⁺ micronuclei in methods for measuring chromosomal instability (as defined by continuous chromosome segregation errors); and (2) illustrates that currently available methods, such as bulk whole genome sequencing, exome sequencing, and single cell RNA sequencing are not only known to be laborious and expensive, but that these methods cannot capture the ongoing rates of chromosome missegregation as there is no correlation between aneuploidy and chromosomal instability itself as shown in FIG. 25I. Without wishing to be bound by theory, this may be the case because aneuploidy is not only a result of the rate of missegregation of chromosomes, but also the product of selection and other factors. Therefore, a method that directly measures chromosomal instability, such as the methods described herein whereby cGAS⁺ micronuclei are used as a surrogate for chromosomal instability, is advantageous. Thus, FIG. 25I shows that CIN and aneuploidy do not necessarily correlate and this highlights an advantage of the methods of the present technology, which provide a measure of CIN without reliance on NexGen techniques to infer aneuploidy.

Accordingly, these results demonstrate the efficacy of methods for measuring the presence of cGAS⁺ micronuclei in tumor samples as a surrogate for detecting chromosomal instability in the tumor samples.

### Example 18: Inhibition of cGAS as a Method for Increasing the Sensitivity of a Tumor to Treatment with STING Activators/Agonists

This example demonstrates the efficacy of cGAS inhibition in a method for increasing tumor sensitivity to treatment with STING activators/agonists.

Chronic activation of STING by constitutively active cGAS (upstream of STING) promotes desensitization to type I interferon signaling downstream of STING, which is necessary for the activation of anti-tumor immunity. The present inventors found that, in cells, alleviating the chronic activation of STING by knockout of cGAS genetically leads to restoration of STING levels and restoration of type I interferon.

Methods. Briefly, wild-type or cGAS-knockout 4T1 tumors were orthotopically transplanted in the mammary fat pad of BALB/c immune competent mice. cGAS was knocked out using CRISPR-Cas9. The mice were treated intratumorally with PBS (vehicle) or the STING agonist, ADU-S100 (10 μg).

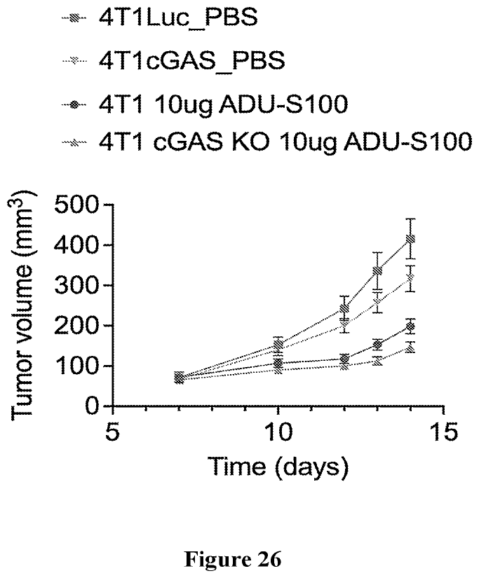

As shown in FIG. 26, tumors that lack cGAS (cGAS KO) are more sensitive to STING agonists than tumors that have active cGAS. This data shows that the most sensitive condition is the one treated with ADU-S100 in the absence of cGAS.

Accordingly, these results demonstrate the efficacy of methods of the present technology for increasing the sensitivity of tumors to treatment and methods for treating cancer by inhibiting cGAS (for example, in a subject for whom a cGAS^{high}STING^{low} tumor has been detected) and subsequently (or sequentially or simultaneously) administering a STING agonist to the subject.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

What is claimed is:

1. A method for detecting chromosomal instability in a tumor in a subject, comprising:
obtaining one or more tissue sections of a tumor sample from a subject; and
measuring the degree of chromosomal instability present in the tumor sample by contacting the tumor sample with an anti-cGAS antibody and measuring the presence of cGAS⁺ micronuclei in the tumor sample.

2. The method of claim 1, wherein measuring the presence of cGAS⁺ micronuclei in the tumor sample comprises one or more of:
quantifying the number of cGAS⁺ micronuclei in a defined high-power field, wherein a high degree of chromosomal instability in the tumor is detected when 5 or more cGAS⁺ micronuclei are present in the high-power field; and
quantifying the fraction of cGAS⁺ micronuclei/primary nuclei, wherein a high degree of chromosomal instability in the tumor is detected when the fraction of cGAS⁺ micronuclei/primary nuclei is 8% or higher.

3. The method of claim 2, wherein the measuring of the presence of cGAS⁺ micronuclei in the tumor sample comprises quantifying the fraction of cGAS⁺ micronuclei/primary nuclei, wherein a high degree of chromosomal instability in the tumor is detected when the fraction of cGAS⁺ micronuclei/primary nuclei is 10% or higher.

4. A method for treating cancer associated with increased cGAS⁺ micronuclei and decreased stimulator of interferon genes (STING) protein expression (cGAS^{high}STING^{low}), in a subject in need thereof, comprising:
detecting an increase in cGAS⁺ micronuclei and a decrease in cancer cell-specific STING protein expression in a tumor sample (cGAS^{high}STING^{low} tumor) obtained from a subject as compared to that observed in a reference sample, thereby detecting a cGAS^{high}STING^{low} tumor; and
administering a therapeutically effective amount of a STING inhibitor to the subject for whom a cGAS^{high}STING^{low} tumor has been detected.

5. A method for selecting a subject for the treatment of cancer with a stimulator of interferon genes (STING) inhibitor, comprising:
detecting an increase in cGAS⁺ micronuclei and a decrease in STING protein expression in a tumor sample (cGAS^{high}STING^{low} tumor) obtained from a subject as compared to that observed in a reference sample; and
selecting the subject for whom a cGAS^{high}STING^{low} tumor sample has been detected as a subject for the treatment of cancer with a STING inhibitor.

6. The method of claim 4 or claim 5, wherein the reference sample is obtained from a healthy control subject, or normal tissue corresponding to the tumor sample, or contains a predetermined level of cGAS⁺ micronuclei and STING protein expression.

7. The method of any one of claims 4-6, wherein detecting cGAS⁺ micronuclei comprises contacting the tumor sample with an anti-cGAS antibody and measuring the presence of cGAS⁺ micronuclei in the tumor sample, and wherein detecting cancer cell-specific STING protein expression comprises contacting the tumor sample with an anti-STING antibody and measuring STING expression levels in the tumor sample.

8. The method of any one of claims 4-7, wherein the STING inhibitor is selected from the group consisting of C-176, C-178, compound H-151, tetrahydroisoquinolone acetic acids, 9-nitrooleate, 10-nitrooleate, nitro conjugated linoleic acid, nitrofurans, Astin C, Astin C analogue M11, and any combination thereof.

9. The method of any one of claims 4-8, further comprising separately, sequentially, or simultaneously administering to the subject one or more immune checkpoint blocking agents selected from the group consisting of anti-PD-L1 antibody, anti-PD-1 antibody, anti-CTLA-4 antibody, ipilimumab, nivolumab, pidilizumab, lambrolizumab, pembrolizumab, atezolizumab, avelumab, durvalumab, MPDL3280A, BMS-936559, MEDI-4736, MSB 00107180, LAG-3, TIM3, B7-H3, B7-H4, TIGIT, AMP-224, MDX-1105, arelumab, tremelimumab, IMP321, MGA271, BMS-986016, lirilumab, urelumab, PF-05082566, IPH2101, MEDI-6469, CP-870,893, Mogamulizumab, Varlilumab, Galiximab, AMP-514, AUNP 12, Indoximod, NLG-919, INCB024360, CD80, CD86, ICOS, DLBCL inhibitors, BTLA, PDR001, and any combination thereof.

10. The method of any one of claims 4-9, wherein the cancer is a solid malignant tumor.

11. The method of claim 10, wherein the solid malignant tumor is selected from the group consisting of melanoma, breast cancer, colorectal cancer, lung cancer, prostate cancer, bladder cancer, pancreatic cancer, ovarian cancer, squamous cell carcinoma of the skin, Merkel cell carcinoma, gastric cancer, liver cancer, thyroid cancer, and sarcoma.

12. The method of any one of claims 4-11, wherein treatment comprises increasing survival, decreasing metastasis, reducing tumor burden, reducing tumor relapse during post-debulking adjuvant chemotherapy in the subject, reducing the number of cancer cells, reducing the tumor size, eradicating the tumor, inhibiting cancer cell infiltration into peripheral organs, inhibiting or stabilizing tumor growth, and/or stabilizing or improving quality of life in the subject.

13. The method of any one of claims 4-12, wherein the subject is a mammal.

14. The method of claim 13, wherein the mammalian subject is a human.

15. A method for increasing tumor sensitivity to treatment with a stimulator of interferon genes (STING) agonist in a subject in need thereof, comprising:
detecting an increase in cGAS⁺ micronuclei and a decrease in STING protein expression in a tumor sample (cGAS^{high}STING^{low} tumor) obtained from a subject as compared to that observed in a reference sample; and
administering a therapeutically effective amount of a cGAS inhibitor to the subject for whom a cGAS^{high}STING^{low} tumor has been detected prior to administering a therapeutically effective amount of a STING agonist to the subject.

16. A method for treating cancer having increased cGAS⁺ micronuclei and decreased stimulator of interferon genes (STING) protein expression in cancer cells (cGAS^{high}STING^{low}), in a subject in need thereof, comprising:
detecting an increase in cGAS⁺ micronuclei and a decrease in cancer cell-specific STING protein expression in a tumor sample (cGAS^{high}STING^{low} tumor) obtained from a subject as compared to that observed in a reference sample, thereby detecting a cGAS^{high}STING^{low} tumor; and
administering a therapeutically effective amount of a cGAS inhibitor to the subject for whom a cGAS^{high}STING^{low} tumor has been detected; and
subsequently administering a STING agonist to the subject for whom a cGAS^{high}STING^{low} tumor has been detected.

17. The method of claim 15 or 16, wherein the reference sample is obtained from a healthy control subject, or normal tissue corresponding to the tumor sample, or contains a predetermined level of cGAS⁺ micronuclei and STING protein expression.

18. The method of any one of claims 15-17, wherein detecting cGAS⁺ micronuclei comprises contacting the tumor sample with an anti-cGAS antibody and measuring the presence of cGAS⁺ micronuclei in the tumor sample, and wherein detecting cancer cell-specific STING protein expression comprises contacting the tumor sample with an anti-STING antibody and measuring STING expression levels in the tumor sample.

19. The method of any one of claims 15-18, wherein the cGAS inhibitor is selected from the group consisting of J014 or analogs thereof, G150 or analogs thereof, RU.521, suramin, PF-06928215, hydroxychloroquine, quinacrin, and any combination thereof.

20. The method of any one of claims 15-19, wherein the STING agonist is selected from the group consisting of c-di-AMP, diABZIs, 3′3′-cGAMP, 2′3′-cGAMP, 5,6-dimethylxanthenone-4-acetic acid (DMXAA), macrocycle-bridged STING agonist E7766, GSK3745417, MK-1454, MK-2118, ADU-S100, SB11285, BMS-98630, and any combination thereof.

21. The method of any one of claims 15-20, wherein the STING agonist is administered within about less than 1 hour, or within about 0 hours, about 24 hours, about 48 hours, about 72 hours, about one week, or about two weeks or more of the cGAS inhibitor.

22. The method of any one of claims 15-21, further comprising separately, sequentially, or simultaneously administering to the subject:
(i) one or more immune checkpoint blocking agents selected from the group consisting of anti-PD-L1 antibody, anti-PD-1 antibody, anti-CTLA-4 antibody, ipilimumab, nivolumab, pidilizumab, lambrolizumab, pembrolizumab, atezolizumab, avelumab, durvalumab, MPDL3280A, BMS-936559, MEDI-4736, MSB 00107180, LAG-3, TIM3, B7-H3, B7-H4, TIGIT, AMP-224, MDX-1105, arelumab, tremelimumab, IMP321, MGA271, BMS-986016, lirilumab, urelumab, PF-05082566, IPH2101, MEDI-6469, CP-870,893, Mogamulizumab, Varlilumab, Galiximab, AMP-514, AUNP 12, Indoximod, NLG-919, INCB024360, CD80, CD86, ICOS, DLBCL inhibitors, BTLA, PDR001, and any combination thereof;
(ii) radiation therapy; and/or
(iii) chemotherapy.

23. The method of any one of claims 15-22, wherein the cancer is a solid malignant tumor.

24. The method of claim 23, wherein the solid malignant tumor is selected from the group consisting of melanoma, breast cancer, colorectal cancer, lung cancer, prostate cancer, bladder cancer, pancreatic cancer, ovarian cancer, squamous cell carcinoma of the skin, Merkel cell carcinoma, gastric cancer, liver cancer, thyroid cancer, and sarcoma.

25. The method of any one of claims 15-24, wherein treatment comprises increasing survival, decreasing local tumor recurrence, decreasing metastasis, reducing tumor burden, reducing tumor relapse during post-debulking adjuvant chemotherapy in the subject, reducing the number of cancer cells, reducing the tumor size, eradicating the tumor, inhibiting cancer cell infiltration into peripheral organs, inhibiting or stabilizing tumor growth, and/or stabilizing or improving quality of life in the subject.

26. The method of any one of claims 15-25, wherein the subject is a mammal.

27. The method of claim 26, wherein the mammalian subject is a human.

28. A method for treating cancer associated with decreased cGAS⁺ micronuclei and increased stimulator of interferon genes (STING) protein expression (cGAS^{low}STING^{high}), in a subject in need thereof, comprising:
detecting a decrease in cGAS⁺ micronuclei and an increase in cancer cell-specific STING protein expression in a tumor sample (cGAS^{low}STING^{high} tumor) obtained from a subject as compared to that observed in a reference sample, thereby detecting a cGAS^{low}STING^{high} tumor; and
administering one or more cancer therapies selected from radiation therapy, chemotherapy, and immunotherapy to the subject for whom a cGAS^{low}STING^{high} tumor has been detected.

29. A method for selecting a subject for the treatment of cancer with a stimulator of interferon genes (STING) inhibitor, comprising:
detecting a decrease in cGAS⁺ micronuclei and an increase in STING protein expression in a tumor sample (cGAS^{low}STING^{high} tumor) obtained from a subject as compared to that observed in a reference sample; and
selecting the subject for whom a cGAS^{low}STING^{high} tumor sample has been detected as a subject for the treatment of cancer with one or more of radiation therapy, chemotherapy, and immunotherapy.

30. The method of claim 28 or claim 29, wherein the immunotherapy comprises administering to the subject an immune checkpoint blocking agent selected from the group consisting of anti-PD-L1 antibody, anti-PD-1 antibody, anti-CTLA-4 antibody, ipilimumab, nivolumab, pidilizumab, lambrolizumab, pembrolizumab, atezolizumab, avelumab, durvalumab, MPDL3280A, BMS-936559, MEDI-4736, MSB 00107180, LAG-3, TIM3, B7-H3, B7-H4, TIGIT, AMP-224, MDX-1105, arelumab, tremelimumab, IMP321, MGA271, BMS-986016, lirilumab, urelumab, PF-05082566, IPH2101, MEDI-6469, CP-870,893, Mogamulizumab, Varlilumab, Galiximab, AMP-514, AUNP 12, Indoximod, NLG-919, INCB024360, CD80, CD86, ICOS, DLBCL inhibitors, BTLA, PDR001, and any combination thereof.

31. The method of any one of claims 28-30, wherein the reference sample is obtained from a healthy control subject, or normal tissue corresponding to the tumor sample, or contains a predetermined level of cGAS⁺ micronuclei and STING protein expression.

32. The method of any one of claims 28-31, wherein detecting cGAS⁺ micronuclei comprises contacting the tumor sample with an anti-cGAS antibody and measuring the presence of cGAS⁺ micronuclei in the tumor sample, and wherein detecting cancer cell-specific STING protein expression comprises contacting the tumor sample with an anti-STING antibody and measuring STING expression levels in the tumor sample.

33. The method of any one of claims 28-32, wherein the cancer is a solid malignant tumor.

34. The method of claim 33, wherein the solid malignant tumor is selected from the group consisting of melanoma, breast cancer, colorectal cancer, lung cancer, prostate cancer, bladder cancer, pancreatic cancer, ovarian cancer, squamous cell carcinoma of the skin, Merkel cell carcinoma, gastric cancer, liver cancer, thyroid cancer, and sarcoma.

35. The method of any one of claims 28-34, wherein treatment comprises increasing survival, decreasing local tumor recurrence, decreasing metastasis, reducing tumor burden, reducing tumor relapse during post-debulking adjuvant chemotherapy in the subject, reducing the number of cancer cells, reducing the tumor size, eradicating the tumor, inhibiting cancer cell infiltration into peripheral organs, inhibiting or stabilizing tumor growth, and/or stabilizing or improving quality of life in the subject.

36. The method of any one of claims 28-35, wherein the subject is a mammal.

37. The method of claim 36, wherein the mammalian subject is a human.

38. A method for treating cancer associated with increased ENPP1 expression in a subject in need thereof, comprising:
detecting the presence or absence of an increased ENPP1 protein expression level in a tumor sample obtained from a subject as compared to that observed in a reference sample; and
administering a therapeutically effective amount of an ENPP1 inhibitor to the subject for whom an increased ENPP1 protein expression level has been detected.

39. A method for treating cancer associated with increased cGAS⁺ micronuclei and increased ENPP1 expression (cGAS^{high}ENPP1^{high}), in a subject in need thereof, comprising:
detecting the presence or absence of an increased level of cGAS⁺ micronuclei and an increased level of ENPP1 protein expression in a tumor sample (cGAS^{high}ENPP1^{high} tumor) obtained from a subject as compared to that observed in a reference sample, thereby detecting a cGAS^{high}ENPP1^{high} tumor; and
administering a therapeutically effective amount of an ENPP1 inhibitor to the subject for whom a cGAS^{high}ENPP1^{high} tumor has been detected.

40. A method for selecting a subject for the treatment of cancer with an ENPP1 inhibitor, comprising:
detecting the presence or absence of an increased ENPP1 protein expression level in a tumor sample obtained from a subject as compared to that observed in a reference sample; and
selecting the subject for whom an increased ENPP1 tumor sample has been detected as a subject for the treatment of cancer with an ENPP1 inhibitor.

41. A method for selecting a subject for the treatment of cancer with an ENPP1 inhibitor comprising:
detecting the presence or absence of an increased level of cGAS⁺ micronuclei and a decreased level of ENPP1 protein expression in a tumor sample (cGAS^{high}ENPP1^{high} tumor) obtained from a subject as compared to that observed in a reference sample, thereby detecting a cGAS^{high}ENPP1^{high} tumor; and
selecting the subject for whom a cGAS^{high}ENPP1^{high} tumor has been detected as a subject for the treatment of cancer with an ENPP1 inhibitor.

42. The method of any one of claims 38-41, wherein the reference sample is obtained from a healthy control subject, or normal tissue corresponding to the tumor sample, or contains a predetermined level of ENPP1 protein expression.

43. The method of claim 41, wherein detecting cGAS⁺ micronuclei comprises contacting the tumor sample with an anti-cGAS antibody and measuring the presence of cGAS⁺ micronuclei in the tumor sample.

44. The method of any one of claims 38-43, wherein detecting ENPP1 protein expression comprises contacting the tumor sample with an anti-ENPP1 antibody and measuring ENPP1 expression levels in the tumor sample.

45. The method of any one of claims 38-44, wherein the ENPP1 inhibitor is selected from the group consisting of α,β-metADP, α,β-metATP, 2-MeSADP, 2-MeSATP, bzATP, γ-S-α,β-metATP derivatives, ARL 67156, α-borano-β, γ-metATP derivatives, diadenosine boranophosphate derivatives, polyoxometalates [TiW11CoO40]⁸⁻, reactive blue 2 (RB2), quinazoline derivative, suramin, heparin, PPADS, biscoumarin derivative, oxadiazole derivatives, quinazoline derivative, triazole derivative, thioacetamide derivative, isoquinoline derivative, thiadiazolopyrimidinone derivative, STF-1084, thiazolobenzimidazolone derivative, sulfamate derivatives, SR 8314, MV626, MAVU-104, and any combination thereof.

46. The method of any one of claims 38-45, further comprising administering a therapeutically effective amount of an NTSE inhibitor.

47. The method of claim 46, wherein the NTSE inhibitor is selected from the group consisting of α,β-methylene-ADP, PSB-12379, PSB-12489, AD680, 4-({5-[4-fluoro-1-(2H-indazol-6-yl)-1H-1,2,3-benzotriazol-6-yl]-1H-pyrazol-1-yl}methyl)benzonitrile, 4-({5-[4-chloro-1-(2H-indazol-6-yl)-1H-1,2,3-benzotriazol-6-yl]-1H-pyrazol-1-yl}methyl)benzonitrile, ESNT-02, ESNT-03, 5-fluorouridine-5′-O-[(phosphonomethyl)phosphonic acid], 4-benzoylcytidine-5′-O-[(phosphonomethyl)phosphonic acid], N⁴-[O-(4-benzyloxy)]-3-methyl-cytidine-5′-O-[(phosphonomethyl)phosphonic acid], N⁴-[O-(naphthalen-2-yl-methoxy)]-cytidine-5′-O-[(phosphonomethyl)phosphonic acid], substituted 5′-aminoadenosine derivatives, APCP, 2-trifluoromethyl-4,6-diarylquinolines, benzothiazine compounds, RR2-4, RR6, RR8-9, RR11, RR16, RR18, RR20-21, pyrazolo[3,4-b]pyridines, pyrrolo[2,3-b]pyridines, pyrido[2,3-d]pyrimidines, benzofuro[3,2-b]pyridines, (E)-N′-(1-(3-(4-fluorophenyl)-5-phenyl-4,5-dihydro-1H-pyrazol-1-yl)ethylidene)isonicotinohydrazide, and any combination thereof.

48. The method of claim 47, wherein the combination of an ENPP1 inhibitor and an NTSE inhibitor has a synergistic effect in the treatment of cancer.

49. The method of any one of claims 38-48, further comprising separately, sequentially, or simultaneously administering radiation therapy, chemotherapy, and/or immunotherapy to the subject.

50. The method of any one of claims 38-49, wherein the cancer is a solid malignant tumor.

51. The method of claim 50, wherein the solid malignant tumor is selected from the group consisting of melanoma, breast cancer, colorectal cancer, lung cancer, prostate cancer, bladder cancer, pancreatic cancer, ovarian cancer, squamous cell carcinoma of the skin, Merkel cell carcinoma, gastric cancer, liver cancer, thyroid cancer, and sarcoma.

52. The method of any one of claims 38-51, wherein treatment comprises increasing survival, decreasing local tumor recurrence, decreasing metastasis, reducing tumor burden, reducing tumor relapse during post-debulking adjuvant chemotherapy in the subject, reducing the number of cancer cells, reducing the tumor size, eradicating the tumor, inhibiting cancer cell infiltration into peripheral organs, inhibiting or stabilizing tumor growth, and/or stabilizing or improving quality of life in the subject.

53. The method of any one of claims 38-52, wherein the subject is a mammal.

54. The method of claim 53, wherein the mammalian subject is a human.

55. A method for treating cancer with an immune checkpoint blockade agent in a subject in need thereof, comprising:
detecting the presence or absence of a low ENPP1 to cGAS expression ratio in a tumor sample obtained from a subject as compared to that observed in a reference sample; and
administering a therapeutically effective amount of one or more immune checkpoint blockade agents to the subject for whom a low ENPP1 to cGAS expression ratio has been detected.

56. The method of claim 55, wherein the reference sample is obtained from a healthy control subject, or normal tissue corresponding to the tumor sample, or contains a predetermined level of ENPP1 protein expression and cGAS⁺ micronuclei.

57. The method of claim 55 or claim 56, wherein detecting ENPP1 protein expression comprises contacting the tumor sample with an anti-ENPP1 antibody and measuring ENPP1 expression levels in the tumor sample, and detecting cGAS⁺ micronuclei comprises contacting the tumor sample with an anti-cGAS antibody and measuring the presence of cGAS⁺ micronuclei in the tumor sample.

58. The method of any one of claims 55-57, wherein the one or more immune checkpoint blocking agents is selected from the group consisting of anti-PD-L1 antibody, anti-PD-1 antibody, anti-CTLA-4 antibody, ipilimumab, nivolumab, pidilizumab, lambrolizumab, pembrolizumab, atezolizumab, avelumab, durvalumab, MPDL3280A, BMS-936559, MEDI-4736, MSB 00107180, LAG-3, TIM3, B7-H3, B7-H4, TIGIT, AMP-224, MDX-1105, arelumab, tremelimumab, IMP321, MGA271, BMS-986016, lirilumab, urelumab, PF-05082566, IPH2101, MEDI-6469, CP-870,893, Mogamulizumab, Varlilumab, Galiximab, AMP-514, AUNP 12, Indoximod, NLG-919, INCB024360, CD80, CD86, ICOS, DLBCL inhibitors, BTLA, PDR001, and any combination thereof.

59. The method of any one of claims 55-58, further comprising separately, sequentially, or simultaneously administering radiation therapy and/or chemotherapy to the subject.

60. The method of any one of claims 55-59, wherein the cancer is a solid malignant tumor.

61. The method of claim 60, wherein the solid malignant tumor is selected from the group consisting of melanoma, breast cancer, colorectal cancer, lung cancer, prostate cancer, bladder cancer, pancreatic cancer, ovarian cancer, squamous cell carcinoma of the skin, Merkel cell carcinoma, gastric cancer, liver cancer, thyroid cancer, and sarcoma.

62. The method of any one of claims 55-61, wherein treatment comprises increasing survival, decreasing local tumor recurrence, decreasing metastasis, reducing tumor burden, reducing tumor relapse during post-debulking adjuvant chemotherapy in the subject, reducing the number of cancer cells, reducing the tumor size, eradicating the tumor, inhibiting cancer cell infiltration into peripheral organs, inhibiting or stabilizing tumor growth, and/or stabilizing or improving quality of life in the subject.

63. The method of any one of claims 55-62, wherein the subject is a mammal.

64. The method of claim 63, wherein the mammalian subject is a human.

65. A method for treating cancer in a subject in need thereof, comprising:
administering a therapeutically effective amount of an ENPP1 inhibitor and an NT5E inhibitor to the subject.

66. The method of claim 65, wherein the ENPP1 inhibitor is selected from the group consisting of α,β-metADP, α,β-metATP, 2-MeSADP, 2-MeSATP, bzATP, γ-S-α,β-metATP derivatives, ARL 67156, α-borano-β, γ-metATP derivatives, diadenosine boranophosphate derivatives, polyoxometalates [TiW11CoO40]⁸⁻, reactive blue 2 (RB2), quinazoline derivative, suramin, heparin, PPADS, biscoumarin derivative, oxadiazole derivatives, quinazoline derivative, triazole derivative, thioacetamide derivative, isoquinoline derivative, thiadiazolopyrimidinone derivative, STF-1084, thiazolobenzimidazolone derivative, sulfamate derivatives, SR 8314, MV626, MAVU-104, and any combination thereof.

67. The method of claim 65 or claim 66, wherein the NT5E inhibitor is selected from the group consisting of α,β-methylene-ADP, PSB-12379, PSB-12489, AD680, 4-({5-[4-fluoro-1-(2H-indazol-6-yl)-1H-1,2,3-benzotriazol-6-yl]-1H-pyrazol-1-yl}methyl)benzonitrile, 4-({5-[4-chloro-1-(2H-indazol-6-yl)-1H-1,2,3-benzotriazol-6-yl]-1H-pyrazol-1-yl}methyl)benzonitrile, ESNT-02, ESNT-03, 5-fluorouridine-5′-O-[(phosphonomethyl)phosphonic acid], 4-benzoylcytidine-5′-O-[(phosphonomethyl)phosphonic acid], N⁴-[O-(4-benzyloxy)]-3-methyl-cytidine-5′-O-[(phosphonomethyl)phosphonic acid], N⁴[O-(naphthalen-2-yl-methoxy)]-cytidine-5′-O-[(phosphonomethyl)phosphonic acid], substituted 5′-aminoadenosine derivatives, APCP, 2-trifluoromethyl-4,6-diarylquinolines, benzothiazine compounds, RR2-4, RR6, RR8-9, RR11, RR16, RR18, RR20-21, pyrazolo[3,4-b]pyridines, pyrrolo[2,3-b]pyridines, pyrido[2,3-d]pyrimidines, benzofuro[3,2-b]pyridines, (E)-N′-(1-(3-(4-fluorophenyl)-5-phenyl-4,5-dihydro-1H-pyrazol-1-yl)ethylidene)isonicotinohydrazide, and any combination thereof.

68. The method of any one of claims 65-67, wherein the combination of an ENPP1 inhibitor and an NT5E inhibitor has a synergistic effect in the treatment of cancer.

69. The method of any one of claims 65-68, further comprising separately, sequentially, or simultaneously administering radiation therapy, chemotherapy, immunotherapy, and/or therapies that induce DNA damage or genomic instability to the subject.

70. The method of any one of claims 65-69, further comprising separately, sequentially, or simultaneously administering to the subject one or more immune checkpoint blocking agents selected from the group consisting of anti-PD-L1 antibody, anti-PD-1 antibody, anti-CTLA-4 antibody, ipilimumab, nivolumab, pidilizumab, lambrolizumab, pembrolizumab, atezolizumab, avelumab, durvalumab, MPDL3280A, BMS-936559, MEDI-4736, MSB 00107180, LAG-3, TIM3, B7-H3, B7-H4, TIGIT, AMP-224, MDX-1105, arelumab, tremelimumab, IMP321, MGA271, BMS-986016, lirilumab, urelumab, PF-05082566, IPH2101, MEDI-6469, CP-870,893, Mogamulizumab, Varlilumab, Galiximab, AMP-514, AUNP 12, Indoximod, NLG-919, INCB024360, CD80, CD86, ICOS, DLBCL inhibitors, BTLA, PDR001, and any combination thereof.

71. The method of any one of claims 65-70, wherein the cancer is a solid malignant tumor.

72. The method of claim 71, wherein the solid malignant tumor is selected from the group consisting of melanoma, breast cancer, colorectal cancer, lung cancer, prostate cancer, bladder cancer, pancreatic cancer, ovarian cancer, squamous cell carcinoma of the skin, Merkel cell carcinoma, gastric cancer, liver cancer, thyroid cancer, and sarcoma.

73. The method of any one of claims 65-72, wherein treatment comprises reducing tumor volume, increasing immune infiltration, decreasing metastasis, treating primary tumors, increasing immune activation against tumors, sensitizing the tumor to immunotherapy, sensitizing the tumor to radiation therapy, sensitizing the tumor to chemotherapy, sensitizing the tumor to therapies that induce DNA damage or genomic instability, increasing survival, decreasing local tumor recurrence, reducing tumor burden, reducing tumor relapse during post-debulking adjuvant chemotherapy in the subject, reducing the number of cancer cells, reducing the tumor size, eradicating the tumor, inhibiting cancer cell infiltration into peripheral organs, inhibiting or stabilizing tumor growth, and/or stabilizing or improving quality of life in the subject.

74. The method of any one of claims 65-73, wherein the subject is a mammal.

75. The method of claim 74, wherein the mammalian subject is a human.

76. A method for treating cancer in a subject in need thereof, comprising:
administering a therapeutically effective amount of a STING agonist and an NT5E inhibitor to the subject.

77. The method of claim 65, wherein the STING agonist is selected from the group consisting of c-di-AMP, c-di-GMP, diABZIs, 3′3′-cGAMP, 2′3′-cGAMP, 5,6-dimethylxanthenone-4-acetic acid (DMXAA), macrocycle-bridged STING agonist E7766, GSK3745417, MK-1454, MX-2118, ADU-S100, SB11285, BMS-98630, and any combination thereof.

78. The method of claim 76 or claim 77, wherein the NT5E inhibitor is selected from the group consisting of α,β-methylene-ADP, PSB-12379, PSB-12489, AD680, 4-({5-[4-fluoro-1-(2H-indazol-6-yl)-1H-1,2,3-benzotriazol-6-yl]-1H-pyrazol-1-yl}methyl)benzonitrile, 4-({5-[4-chloro-1-(2H-indazol-6-yl)-1H-1,2,3-benzotriazol-6-yl]-1H-pyrazol-1-yl}methyl)benzonitrile, ESNT-02, ESNT-03, 5-fluorouridine-5′-O-[(phosphonomethyl)phosphonic acid], 4-benzoylcytidine-5′-O-[(phosphonomethyl)phosphonic acid], N⁴-[O-(4-benzyloxy)]-3-methyl-cytidine-5′-O-[(phosphonomethyl)phosphonic acid], N⁴-[O-(naphthalen-2-yl-methoxy)]-cytidine-5′-O-[(phosphonomethyl)phosphonic acid], substituted 5′-aminoadenosine derivatives, APCP, 2-trifluoromethyl-4,6-diarylquinolines, benzothiazine compounds, RR2-4, RR6, RR8-9, RR11, RR16, RR18, RR20-21, pyrazolo[3,4-b]pyridines, pyrrolo[2,3-b]pyridines, pyrido[2,3-d]pyrimidines, benzofuro[3,2-b]pyridines, (E)-N′-(1-(3-(4-fluorophenyl)-5-phenyl-4,5-dihydro-1H-pyrazol-1-yl)ethylidene)isonicotinohydrazide, and any combination thereof.

79. The method of any one of claims 76-78, wherein the combination of STING agonist and an NT5E inhibitor has a synergistic effect in the treatment of cancer.

80. The method of any one of claims 76-79, further comprising separately, sequentially, or simultaneously administering radiation therapy, chemotherapy, immunotherapy, and/or therapies that induce DNA damage or genomic instability to the subject.

81. The method of any one of claims 76-80, further comprising separately, sequentially, or simultaneously administering to the subject one or more immune checkpoint blocking agents selected from the group consisting of anti-PD-L1 antibody, anti-PD-1 antibody, anti-CTLA-4 antibody, ipilimumab, nivolumab, pidilizumab, lambrolizumab, pembrolizumab, atezolizumab, avelumab, durvalumab, MPDL3280A, BMS-936559, MEDI-4736, MSB 00107180, LAG-3, TIM3, B7-H3, B7-H4, TIGIT, AMP-224, MDX-1105, arelumab, tremelimumab, IMP321, MGA271, BMS-986016, lirilumab, urelumab, PF-05082566, IPH2101, MEDI-6469, CP-870,893, Mogamulizumab, Varlilumab, Galiximab, AMP-514, AUNP 12, Indoximod, NLG-919, INCB024360, CD80, CD86, ICOS, DLBCL inhibitors, BTLA, PDR001, and any combination thereof.

82. The method of any one of claims 76-81, wherein the cancer is a solid malignant tumor.

83. The method of claim 82, wherein the solid malignant tumor is selected from the group consisting of melanoma, breast cancer, colorectal cancer, lung cancer, prostate cancer, bladder cancer, pancreatic cancer, ovarian cancer, squamous cell carcinoma of the skin, Merkel cell carcinoma, gastric cancer, liver cancer, thyroid cancer, and sarcoma.

84. The method of any one of claims 76-83, wherein treatment comprises reducing tumor volume, increasing immune infiltration, decreasing metastasis, treating primary tumors, increasing immune activation against tumors, sensitizing the tumor to immunotherapy, sensitizing the tumor to radiation therapy, sensitizing the tumor to chemotherapy, sensitizing the tumor to therapies that induce DNA damage or genomic instability, increasing survival, decreasing local tumor recurrence, reducing tumor burden, reducing tumor relapse during post-debulking adjuvant chemotherapy in the subject, reducing the number of cancer cells, reducing the tumor size, eradicating the tumor, inhibiting cancer cell infiltration into peripheral organs, inhibiting or stabilizing tumor growth, and/or stabilizing or improving quality of life in the subject.

85. The method of any one of claims 76-84, wherein the subject is a mammal.

86. The method of claim 85, wherein the mammalian subject is a human.

87. The method of any one of claim 7, 18, 32, 43, or 57, wherein measuring the presence of cGAS⁺ micronuclei in the tumor sample comprises one or more of:
quantifying the number of cGAS⁺ micronuclei in a defined high-power field;
performing a semi-quantitative assessment; and
measuring cGAS⁺ micronuclei as a fraction of cGAS⁺ micronuclei/primary nuclei.
